(19) United States
(12) Patent Application Publication
(10) Pub. No.: US 2026/0207739 A1
(43) Pub. Date: Jul. 23, 2026

(54) COMBINATION THERAPY FOR TREATING CANCER

(71) Applicant: Gilead Sciences, Inc., Foster City, CA (US)

(72) Inventors: Jamie G. Bates, Burlingame, CA (US); Heike Fuhrmann-Stroissnigg, Foster City, CA (US); Priya K.G. Turumella, San Francisco, CA (US)

(21) Appl. No.: 19/141,926

(22) PCT Filed: Dec. 18, 2023
(86) PCT No.: PCT/US2023/084512

§ 371 Date: Jun. 20, 2025

Related U.S. Application Data
(60) Provisional application No. 63/476,567, filed on Dec. 21, 2022.

(51) Int. Cl. A61K 39/395; A61K 31/4375; A61K 31/498; A61K 31/517; A61P 35/00

(52) U.S. Cl. CPC A61K 39/3955; A61K 31/4375; A61K 31/498; A61K 31/517; A61P 35/00

(57) ABSTRACT

The present disclosure relates to methods of treating cancer in a subject (e.g., a human patient) comprising co-administering to the subject an effective amount of an integrin activity modulator; and a PD-1 inhibitor (e.g., an anti-PD-1 antibody) or PD-L1 inhibitor (e.g., an anti-PD-L1 antibody). In some embodiments the cancer is pancreatic cancer. In some embodiments the cancer is breast cancer.

### CROSS-REFERENCE TO RELATED APPLICATIONS

This application claims the benefit under 35 U.S.C. § 119(e) of U.S. Provisional Application No. 63/476,567, filed on Dec. 21, 2022, which are hereby incorporated herein by reference in its entirety for all purposes.

### FIELD

The present disclosure relates to methods of treating cancer in a subject (e.g., a human patient) comprising co-administering to the subject an effective amount of an integrin activity modulator; and a PD-1 inhibitor (e.g., an anti-PD-1 antibody) or PD-L1 inhibitor (e.g., an anti-PD-L1 antibody). In some embodiments the cancer is pancreatic cancer. In some embodiments the cancer is breast cancer.

### BACKGROUND

Integrins are a superfamily of cell adhesion receptors and have been found to be involved in cancer (progression, metastasis, angiogenesis), sepsis, fibrosis, and viral infections. Over the last decades, integrins have been investigated as therapeutic targets for cancer, including numerous clinical trials with integrin-targeted therapies. However, there is no approved integrin-targeted therapy for the treatment of cancer.

Immune checkpoint inhibitors have reinvigorated clinical development interest in anticancer immunotherapy. The latter relies on therapeutic modulation of the tumor microenvironment or other aspects of the immune system to overcome mechanisms of immune suppression that a tumor elicits on the host immune system. Despite their proven benefit in numerous tumor types as evidenced by the approvals of nivolumab, pembrolizumab, atezolizumab, ipilimumab, and others, many patients either remain refractory to currently available therapy, or relapse following a response to available therapy. Thus, a medical need remains for the development of novel agents and combinations of agents for the treatment of many types of cancers, such as pancreatic cancer and breast cancer.

### SUMMARY

Disclosed herein are methods of treating, mitigating, reducing, preventing or delaying the recurrence or metastasis of cancer in a subject comprising co-administering to the subject an effective amount of (a) an integrin activity modulator; and (b) a PD-1 inhibitor or PD-L1 inhibitor.

Further disclosed herein are methods for eliciting an immune response to cancer in a subject comprising co-administering to the subject an effective amount of (a) an integrin activity modulator; and (b) a PD-1 inhibitor or PD-L1 inhibitor.

Disclosed herein are methods of treating, mitigating, reducing, preventing or delaying the recurrence or metastasis of cancer in a subject comprising co-administering to the subject an effective amount of (a) an integrin activity modulator; and (b) a PD-1 inhibitor or PD-L1 inhibitor, wherein (i) the integrin activity modulator is selected from bexotegrast (i.e., PLN-74809), PLN-1474, and PLN-1561; or (ii) the PD-1 inhibitor or PD-L1 inhibitor is selected from atezolizumab, durvalumab, MK-7684A, nivolumab, pembrolizumab, and zimberelimab.

Disclosed herein are methods for eliciting an immune response to cancer in a subject comprising co-administering to the subject an effective amount of (a) an integrin activity modulator; and (b) a PD-1 inhibitor or PD-L1 inhibitor, wherein (i) the integrin activity modulator is selected from bexotegrast (i.e., PLN-74809), PLN-1474, and PLN-1561; or (ii) the PD-1 inhibitor or PD-L1 inhibitor is selected from atezolizumab, durvalumab, MK-7684A, nivolumab, pembrolizumab, and zimberelimab.

Disclosed herein are kits for use as a medicament, wherein the kit comprises (a) an integrin activity modulator; and (b) a PD-1 inhibitor or PD-L1 inhibitor.

Disclosed herein are kits for use in the treatment, mitigation, reduction, prevention, or delay of the recurrence or metastasis of cancer, wherein the kit comprises (a) an integrin activity modulator; and (b) a PD-1 inhibitor or PD-L1 inhibitor.

Disclosed herein are kits for use as a medicament, wherein the kit comprises (a) an integrin activity modulator; and (b) a PD-1 inhibitor or PD-L1 inhibitor, wherein (i) the integrin activity modulator is selected from bexotegrast (i.e., PLN-74809), PLN-1474, and PLN-1561; or (ii) the PD-1 inhibitor or PD-L1 inhibitor is selected from atezolizumab, durvalumab, MK-7684A, nivolumab, pembrolizumab, and zimberelimab.

Disclosed herein are kits for use in the treatment, mitigation, reduction, prevention, or delay of the recurrence or metastasis of cancer, wherein the kit comprises (a) an integrin activity modulator; and (b) a PD-1 inhibitor or PD-L1 inhibitor, wherein (i) the integrin activity modulator is selected from bexotegrast (i.e., PLN-74809), PLN-1474, and PLN-1561; or (ii) the PD-1 inhibitor or PD-L1 inhibitor is selected from atezolizumab, durvalumab, MK-7684A, nivolumab, pembrolizumab, and zimberelimab.

In some embodiments, the integrin activity modulator is selected from an integrin-activating agent, an integrin-inhibiting agent, or an agent that is capable of interacting with one or more integrins.

In some embodiments, the integrin activity modulator is a small molecule or a large molecule. In some embodiments, the large molecule is an antibody or antibody fragment. In some embodiments, the large molecule is an inhibitory peptide or peptide antagonist.

In some embodiments, the integrin activity modulator activates, inhibits, or interacts with an integrin alpha (α) subunit. In some embodiments, the integrin a subunit is selected from α5, αV, α8, and αIIb. In some embodiments, the integrin activity modulator activates, inhibits, or interacts with the αV subunit. In some embodiments, the integrin activity modulator is an anti-αV antibody. In some embodiments, the anti-αV antibody is selected from abituzumab and intetumumab.

In some embodiments, the integrin activity modulator is an iRGD peptide. In some embodiments, the iRGD peptide is CEND-1.

In some embodiments, the integrin activity modulator activates, inhibits, or interacts with an integrin beta (β) subunit. In some embodiments, the β subunit is selected from β1, β3, β5, and β8. In some embodiments, the integrin activity modulator activates, inhibits, or interacts with β1. In some embodiments, the integrin activity modulator is an anti-β1 antibody. In some embodiments, the anti-β1 antibody is OS2966.

In some embodiments, the integrin activity modulator activates, inhibits, or interacts with β5. In some embodiments, the integrin activity modulator is an anti-β5 antibody. In some embodiments, the anti-β5 antibody is ALULA.

In some embodiments, the integrin activity modulator activates, inhibits, or interacts with an RGD-binding integrin. In some embodiments, RGD-binding integrin is selected from α5β1, αVβ1, αVβ3, αVβ5, αVβ6, αVβ8, α8β1, and αIIbβ3.

In some embodiments, the integrin activity modulator activates, inhibits, or interacts with α5β1. In some embodiments, the integrin activity modulator is selected from AG-73305, AP25, AMEP, ATN-161, F-200 (Eos), JSM-6427, liposomal contortrostatin, PF-04605412, bexotegrast (i.e., PLN-74809), and SJ-749.

In some embodiments, the integrin activity modulator activates, inhibits, or interacts with αVβ1. In some embodiments, the integrin activity modulator is selected from bexotegrast (i.e., PLN-74809), IDL-2965, PLN-1474, PLN-1561, and volociximab. In some embodiments, the integrin activity modulator is selected from bexotegrast (i.e., PLN-74809), PLN-1474, and PLN-1561. In some embodiments, the integrin activity modulator is bexotegrast. In some embodiments, the integrin activity modulator is PLN-1474. In some embodiments, the integrin activity modulator is PLN-1561.

In some embodiments, the integrin activity modulator activates, inhibits, or interacts with αVβ3. In some embodiments, the integrin activity modulator is selected from AP25, B-1451, BS-1417, cilengitide, CT-6725, efatucizumab, etaracizumab, FF-10158, HM-3, IPS-02001, IPS-02101, IPS-05002, JNJ-26076713, MK-0429, NTA-1, NVX-188, Ocurin, ProAgio, PS-388023, RP-697, RP-748, SB-273005, Sch-221153, SC-69000, SD-983, SF-0166, SM-256, SOM-0777, SQ-885, TSRI-265, VIP-236, vitaxin, VPI-2690B, and XT-199.

In some embodiments, the integrin activity modulator activates, inhibits, or interacts with αVβ5. In some embodiments, the integrin activity modulator is selected from ALULA, SNAKA-51, and STX-200.

In some embodiments, the integrin activity modulator activates, inhibits, or interacts with αVβ6. In some embodiments, the integrin activity modulator is selected from 264-RAD, A20FMDV2, abituzumab, ADVCA0848, bexotegrast (i.e., PLN-74809), CRB-602, GSK-3008348, IDL-2965, intetumumab, MORF-627, MORF-720, PLN-1177, PLN-1561, PLN-1705, SGN-B6A, and STX-100.

In some embodiments, the integrin activity modulator activates, inhibits, or interacts with αVβ8. In some embodiments, the integrin activity modulator is selected from CRB-601 and PF-06940434.

In some embodiments, the integrin activity modulator activates, inhibits, or interacts with α8β1. In some embodiments, the integrin activity modulator is an anti-α8β1 antibody. In some embodiments, the anti-α8β1 antibody is an anti-α8β1 antibody disclosed in WO2011049082.

In some embodiments, the integrin activity modulator activates, inhibits, or interacts with αIIbβ3. In some embodiments, the integrin activity modulator is selected from eptifibatide, RUC-1, Tc-99m-P280, tirofiban, zalunfiban, and an antibody that interacts with αIIbβ3.

In some embodiments, the PD-1 inhibitor or PD-L1 inhibitor is an anti-PD-1 antibody. In some embodiments, the anti-PD-1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), PF-06801591, MGD-019 (PD-1/CTLA4), KN-046 (PD-1/CTLA4), XmAb-20717 (PD-1/CTLA4), AK-104 (CTLA4/PD-1), and MEDI-5752 (CTLA4/PD-1). In some embodiments, the anti-PD-1 antibody is zimberelimab.

In some embodiments, the PD-1 inhibitor or PD-L1 inhibitor is an anti-PD-L1 antibody. In some embodiments, the PD-L1 inhibitor is selected from the group consisting of atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR).

In some embodiments, the PD-L1 inhibitor is a small molecule inhibitor. In some embodiments, the small molecule PD-L1 inhibitor is selected from the group consisting of CA-170, GS-4224, GS-4416, INCB99280, INCB99318, and lazertinib.

In some embodiments, the PD-1 inhibitor or PD-L1 inhibitor is atezolizumab.

In some embodiments, the PD-1 inhibitor or PD-L1 inhibitor is durvalumab.

In some embodiments, the PD-1 inhibitor or PD-L1 inhibitor is MK-7684A.

In some embodiments, the PD-1 inhibitor or PD-L1 inhibitor is nivolumab.

In some embodiments, the PD-1 inhibitor or PD-L1 inhibitor is pembrolizumab.

In some embodiments, the PD-1 inhibitor or PD-L1 inhibitor is zimberelimab.

In some embodiments, the cancer comprises a solid tumor. In some embodiments, the solid tumor is located in or arising from a tissue or organ selected from the group consisting of:
bone (e.g., adamantinoma, aneurysmal bone cysts, angiosarcoma, chondroblastoma, chondroma, chondromyxoid fibroma, chondrosarcoma, chordoma, dedifferentiated chondrosarcoma, enchondroma, epithelioid hemangioendothelioma, fibrous dysplasia of the bone, giant cell tumour of bone, haemangiomas and related lesions, osteoblastoma, osteochondroma, osteosarcoma, osteoid osteoma, osteoma, periosteal chondroma, Desmoid tumor, Ewing sarcoma);
lips and oral cavity (e.g., odontogenic ameloblastoma, oral leukoplakia, oral squamous cell carcinoma, primary oral mucosal melanoma); salivary glands (e.g., pleomorphic salivary gland adenoma, salivary gland adenoid cystic carcinoma, salivary gland mucoepidermoid carcinoma, salivary gland Warthin's tumors);
esophagus (e.g., Barrett's esophagus, dysplasia and adenocarcinoma);
gastrointestinal tract, including stomach (e.g., gastric adenocarcinoma, primary gastric lymphoma, gastrointestinal stromal tumors (GISTs), metastatic deposits, gastric carcinoids, gastric sarcomas, neuroendocrine carcinoma, gastric primary squamous cell carcinoma, gastric adenoacanthomas), intestines and smooth muscle (e.g., intravenous leiomyomatosis), colon (e.g., colorectal adenocarcinoma), rectum, anus;
pancreas (e.g., serous neoplasms, including microcystic or macrocystic serous cystadenoma, solid serous cystadenoma, Von Hippel-Landau (VHL)-associated serous cystic neoplasm, serous cystadenocarcinoma; mucinous cystic neoplasms (MCN), intraductal papillary mucinous neoplasms (IPMN), intraductal oncocytic papillary neoplasms (IOPN), intraductal tubular neoplasms, cystic acinar neoplasms, including acinar cell cystadenoma, acinar cell cystadenocarcinoma, pancreatic adenocarcinoma, invasive pancreatic ductal adenocarcinomas, including tubular adenocarcinoma, adenosquamous carcinoma, colloid carcinoma, medullary carcinoma, hepatoid carcinoma, signet ring cell carcinoma, undifferentiated carcinoma, undifferentiated carcinoma with osteoclast-like giant cells, acinar cell carcinoma, neuroendocrine neoplasms, neuroendocrine microadenoma, neuroendocrine tumors (NET), neuroendocrine carcinoma (NEC), including small cell or large cell NEC, insulinoma, gastrinoma, glucagonoma, serotonin-producing NET, somatostatinoma, VIPoma, solid-pseudopapillary neoplasms (SPN), pancreatoblastoma);
gall bladder (e.g., carcinoma of the gallbladder and extrahepatic bile ducts, intrahepatic cholangiocarcinoma);
neuro-endocrine (e.g., adrenal cortical carcinoma, carcinoid tumors, phaeochromocytoma, pituitary adenomas);
thyroid (e.g., anaplastic (undifferentiated) carcinoma, medullary carcinoma, oncocytic tumors, papillary carcinoma, adenocarcinoma);
liver (e.g., adenoma, combined hepatocellular and cholangiocarcinoma, fibrolamellar carcinoma, hepatoblastoma, hepatocellular carcinoma, mesenchymal, nested stromal epithelial tumor, undifferentiated carcinoma; hepatocellular carcinoma, intrahepatic cholangiocarcinoma, bile duct cystadenocarcinoma, epithelioid hemangioendothelioma, angiosarcoma, embryonal sarcoma, rhabdomyosarcoma, solitary fibrous tumor, teratoma, York sac tumor, carcinosarcoma, rhabdoid tumor);
kidney (e.g., ALK-rearranged renal cell carcinoma, chromophobe renal cell carcinoma, clear cell renal cell carcinoma, clear cell sarcoma, metanephric adenoma, metanephric adenofibroma, mucinous tubular and spindle cell carcinoma, nephroma, nephroblastoma (Wilms tumor), papillary adenoma, papillary renal cell carcinoma, renal oncocytoma, renal cell carcinoma, succinate dehydrogenase-deficient renal cell carcinoma, collecting duct carcinoma);
breast (e.g., invasive ductal carcinoma, including without limitation, acinic cell carcinoma, adenoid cystic carcinoma, apocrine carcinoma, cribriform carcinoma, glycogen-rich/clear cell, inflammatory carcinoma, lipid-rich carcinoma, medullary carcinoma, metaplastic carcinoma, micropapillary carcinoma, mucinous carcinoma, neuroendocrine carcinoma, oncocytic carcinoma, papillary carcinoma, sebaceous carcinoma, secretory breast carcinoma, tubular carcinoma; lobular carcinoma, including without limitation, pleomorphic carcinoma, signet ring cell carcinoma);
peritoneum (e.g., mesothelioma; primary peritoneal cancer);
female sex organ tissues, including ovary (e.g., choriocarcinoma, epithelial tumors, germ cell tumors, sex cord-stromal tumors), Fallopian tubes (e.g., serous adenocarcinoma, mucinous adenocarcinoma, endometrioid adenocarcinoma, clear cell adenocarcinoma, transitional cell carcinoma, squamous cell carcinoma, undifferentiated carcinoma, Müllerian tumors, adenosarcoma, leiomyosarcoma, teratoma, germ cell tumors, choriocarcinoma, trophoblastic tumors), uterus (e.g., carcinoma of the cervix, endometrial polyps, endometrial hyperplasia, intraepithelial carcinoma (EIC), endometrial carcinoma (e.g., endometrioid carcinoma, serous carcinoma, clear cell carcinoma, mucinous carcinoma, squamous cell carcinoma, transitional carcinoma, small cell carcinoma, undifferentiated carcinoma, mesenchymal neoplasia), leiomyoma (e.g., endometrial stromal nodule, leiomyosarcoma, endometrial stromal sarcoma (ESS), mesenchymal tumors), mixed epithelial and mesenchymal tumors (e.g., adenofibroma, carcinofibroma, adenosarcoma, carcinosarcoma (malignant mixed mesodermal sarcoma—MMMT)), endometrial stromal tumors, endometrial malignant mullerian mixed tumours, gestational trophoblastic tumors (partial hydatiform mole, complete hydatiform mole, invasive hydatiform mole, placental site tumour)), vulva, vagina;
male sex organ tissues, including prostate, testis (e.g., germ cell tumors, spermatocytic seminoma), penis;
bladder (e.g., squamous cell carcinoma, urothelial carcinoma, bladder urothelial carcinoma);
brain, (e.g., gliomas (e.g., astrocytomas, including non-infiltrating, low-grade, anaplastic, glioblastomas; oligodendrogliomas, ependymomas), meningiomas, gangliogliomas, schwannomas (neurilemmomas), craniopharyngiomas, chordomas, Non-Hodgkin lymphomas (NHLs), indolent non-Hodgkin's lymphoma (iNHL), refractory iNHL, pituitary tumors;
eye (e.g., retinoma, retinoblastoma, ocular melanoma, posterior uveal melanoma, iris hamartoma);
head and neck (e.g., nasopharyngeal carcinoma, Endolymphatic Sac Tumor (ELST), epidermoid carcinoma, laryngeal cancers including squamous cell carcinoma (SCC) (e.g., glottic carcinoma, supraglottic carcinoma, subglottic carcinoma, transglottic carcinoma), carcinoma in situ, verrucous, spindle cell and basaloid SCC, undifferentiated carcinoma, laryngeal adenocarcinoma, adenoid cystic carcinoma, neuroendocrine carcinomas, laryngeal sarcoma), head and neck paragangliomas (e.g., carotid body, jugulotympanic, vagal);
thymus (e.g., thymoma);
heart (e.g., cardiac myxoma);
lung (e.g., small cell carcinoma (SCLC), non-small cell lung carcinoma (NSCLC), including squamous cell carcinoma (SCC), adenocarcinoma and large cell carcinoma, carcinoids (typical or atypical), carcinosarcomas, pulmonary blastomas, giant cell carcinomas, spindle cell carcinomas, pleuropulmonary blastoma);
lymph (e.g., lymphomas, including Hodgkin's lymphoma, non-Hodgkin's lymphoma (NHL), indolent non-Hodgkin's lymphoma (iNHL), refractory iNHL, Epstein-Barr virus (EBV)-associated lymphoproliferative diseases, including B cell lymphomas and T cell lymphomas (e.g., Burkitt lymphoma; large B cell lymphoma, diffuse large B-cell lymphoma (DLBCL), mantle cell lymphoma, indolent B-cell lymphoma, low grade B cell lymphoma, fibrin-associated diffuse large cell lymphoma; primary effusion lymphoma; plasmablastic lymphoma; extranodal NK/T cell lymphoma, nasal type; peripheral T cell lymphoma, cutaneous T cell lymphoma, angioimmunoblastic T cell lymphoma; follicular T cell lymphoma; systemic T cell lymphoma), lymphangioleiomyomatosis);
central nervous system (CNS) (e.g., gliomas including astrocytic tumors (e.g., pilocytic astrocytoma, pilomyxoid astrocytoma, subependymal giant cell astrocytoma, pleomorphic xanthoastrocytoma, diffuse astrocytoma, fibrillary astrocytoma, gemistocytic astrocytoma, protoplasmic astrocytoma, anaplastic astrocytoma, glioblastoma (e.g., giant cell glioblastoma, gliosarcoma, glioblastoma multiforme) and gliomatosis cerebri), oligodendroglial tumors (e.g., oligodendroglioma, anaplastic oligodendroglioma), oligoastrocytic tumors (e.g., oligoastrocytoma, anaplastic oligoastrocytoma), ependymal tumors (e.g., subependymom, myxopapillary ependymoma, ependymomas (e.g., cellular, papillary, clear cell, tanycytic), anaplastic ependymoma), optic nerve glioma, and non-gliomas (e.g., choroid plexus tumors, neuronal and mixed neuronal-glial tumors, pineal region tumors, embryonal tumors, medulloblastoma, meningeal tumors, primary CNS lymphomas, germ cell tumors, Pituitary adenomas, cranial and paraspinal nerve tumors, stellar region tumors); neurofibroma, meningioma, peripheral nerve sheath tumors, peripheral neuroblastic tumours (including without limitation neuroblastoma, ganglioneuroblastoma, ganglioneuroma), trisomy 19 ependymoma);
neuroendocrine tissues (e.g., paraganglionic system including adrenal medulla (pheochromocytomas) and extra-adrenal paraganglia ((extra-adrenal) paragangliomas);
skin (e.g., clear cell hidradenoma, cutaneous benign fibrous histiocytomas, cylindroma, hidradenoma, melanoma (including cutaneous melanoma, mucosal melanoma), pilomatricoma, Spitz tumors); and
soft tissues (e.g., aggressive angiomyxoma, alveolar rhabdomyosarcoma, alveolar soft part sarcoma, angiofibroma, angiomatoid fibrous histiocytoma, synovial sarcoma, biphasic synovial sarcoma, clear cell sarcoma, dermatofibrosarcoma protuberans, desmoid-type fibromatosis, small round cell tumor, desmoplastic small round cell tumor, elastofibroma, embryonal rhabdomyosarcoma, Ewing's tumors/primitive neurectodermal tumors (PNET), extraskeletal myxoid chondrosarcoma, extraskeletal osteosarcoma, paraspinal sarcoma, inflammatory myofibroblastic tumor, lipoblastoma, lipoma, chondroid lipoma, liposarcoma/malignant lipomatous tumors, liposarcoma, myxoid liposarcoma, fibromyxoid sarcoma, lymphangioleiomyoma, malignant myoepithelioma, malignant melanoma of soft parts, myoepithelial carcinoma, myoepithelioma, myxoinflammatory fibroblastic sarcoma, undifferentiated sarcoma, pericytoma, rhabdomyosarcoma, non-rhabdomyosarcoma soft tissue sarcoma (NRSTS), soft tissue leiomyosarcoma, undifferentiated sarcoma, well-differentiated liposarcoma.

In some embodiments, the cancer is selected from breast cancer or pancreatic cancer.

In some embodiments, the cancer is breast cancer. In some embodiments, the breast cancer is triple-negative breast cancer (TNBC), HR+/HER2− breast cancer (HR+/HER2− BC), HR+/HER2low breast cancer (HR+/HER2low BC).

In some embodiments, the cancer is pancreatic cancer. In some embodiments, the pancreatic cancer is pancreatic ductal adenocarcinoma (PDAC).

In some embodiments, the cancer is metastatic.

In some embodiments, the subject is human.

In some embodiments, the subject is treatment naïve.

In some embodiments, the subject has received at least one previous anticancer treatment selected from the group consisting of surgery, radiation therapy, chemotherapy, or checkpoint inhibitor therapy.

In some embodiments, the cancer has progressed or recurred following an anticancer treatment selected from the group consisting of surgery, radiation therapy, chemotherapy, or checkpoint inhibitor therapy.

In some embodiments, the cancer is resistant or refractory to checkpoint inhibitor therapy.

In some embodiments, the checkpoint inhibitor therapy comprises an anti-PD-1 antibody or anti-PD-L1 antibody. In some embodiments, the anti-PD-1 antibody or anti-PD-L1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), MGD-019 (PD-1/CTLA4), KN-046 (PD-1/CTLA4), XmAb-20717 (PD-1/CTLA4), AK-104 (CTLA4/PD-1), MEDI-5752 (CTLA4/PD-1), atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, atezolizumab, avelumab, envafolimab, durvalumab, cosibelimab, lodapolimab, garivulimab, envafolimab, opucolimab, and manelimab. In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, and vopratelimab.

In some embodiments, the chemotherapy is selected from the group consisting of 5-FU, leucovorin, oxaliplatin, irinotecan, gemcitabine, nab-paclitaxel (Abraxane®), FOLFIRINOX, and combinations thereof. In some embodiments, therapeutic agents used to treat pancreatic cancer comprise a combination of (a) 5-FU+leucovorin+oxaliplatin+irinotecan, 5-FU+nanoliposomal irinotecan, leucovorin+nanoliposomal irinotecan, and gemcitabine+nab-paclitaxel.

In some embodiments, the method further comprises co-administering an additional therapeutic agent or therapeutic modality.

Disclosed herein are methods of treating, mitigating, reducing, preventing or delaying the recurrence or metastasis of pancreatic cancer or breast cancer in a subject comprising co-administering to the subject an effective amount of (a) an integrin activity modulator selected from bexotegrast (i.e., PLN-74809), PLN-1474, and PLN-1561; and (b) a PD-1 inhibitor or PD-L1 inhibitor selected from atezolizumab, durvalumab, MK-7684A, nivolumab, pembrolizumab, and zimberelimab.

Disclosed herein are methods for eliciting an immune response to pancreatic cancer or breast cancer in a subject comprising co-administering to the subject an effective amount of (a) an integrin activity modulator selected from bexotegrast (i.e., PLN-74809), PLN-1474, and PLN-1561; and (b) a PD-1 inhibitor or PD-L1 inhibitor selected from atezolizumab, durvalumab, MK-7684A, nivolumab, pembrolizumab, and zimberelimab.

Disclosed herein are kits for use as a medicament, wherein the kit comprises (a) an integrin activity modulator selected from bexotegrast (i.e., PLN-74809), PLN-1474, and PLN-1561; and (b) a PD-1 inhibitor or PD-L1 inhibitor selected from atezolizumab, durvalumab, MK-7684A, nivolumab, pembrolizumab, and zimberelimab.

Disclosed herein are kits for use in the treatment, mitigation, reduction, prevention, or delay of the recurrence or metastasis of pancreatic cancer or breast cancer, wherein the kit comprises (a) an integrin activity modulator selected from bexotegrast (i.e., PLN-74809), PLN-1474, and PLN-1561; and (b) a PD-1 inhibitor or PD-L1 inhibitor selected from atezolizumab, durvalumab, MK-7684A, nivolumab, pembrolizumab, and zimberelimab.

In some embodiments, the integrin activity modulator is bexotegrast.

In some embodiments, the integrin activity modulator is PLN-1474.

In some embodiments, the integrin activity modulator is PLN-1561.

In some embodiments, the PD-1 inhibitor or PD-L1 inhibitor is atezolizumab.

In some embodiments, the PD-1 inhibitor or PD-L1 inhibitor is durvalumab.

In some embodiments, the PD-1 inhibitor or PD-L1 inhibitor is MK-7684A.

In some embodiments, the PD-1 inhibitor or PD-L1 inhibitor is nivolumab.

In some embodiments, the PD-1 inhibitor or PD-L1 inhibitor is pembrolizumab.

In some embodiments, the PD-1 inhibitor or PD-L1 inhibitor is zimberelimab.

In some embodiments, the breast cancer is triple-negative breast cancer (TNBC), HR+/HER2− breast cancer (HR+/HER2− BC), HR+/HER2low breast cancer (HR+/HER2low BC).

In some embodiments, the pancreatic cancer is pancreatic ductal adenocarcinoma (PDAC).

In some embodiments, the cancer is metastatic.

In some embodiments, the subject is human.

In some embodiments, the subject is treatment naïve.

In some embodiments, the subject has received at least one previous anticancer treatment selected from the group consisting of surgery, radiation therapy, chemotherapy, or checkpoint inhibitor therapy.

In some embodiments, the cancer has progressed or recurred following an anticancer treatment selected from the group consisting of surgery, radiation therapy, chemotherapy, or checkpoint inhibitor therapy.

In some embodiments, the cancer is resistant or refractory to checkpoint inhibitor therapy.

In some embodiments, the checkpoint inhibitor therapy comprises an anti-PD-1 antibody or anti-PD-L1 antibody. In some embodiments, the anti-PD-1 antibody or anti-PD-L1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), MGD-019 (PD-1/CTLA4), KN-046 (PD-1/CTLA4), XmAb-20717 (PD-1/CTLA4), AK-104 (CTLA4/PD-1), MEDI-5752 (CTLA4/PD-1), atezolizumab, Pelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, atezolizumab, avelumab, envafolimab, durvalumab, cosibelimab, lodapolimab, garivulimab, envafolimab, opucolimab, and manelimab. In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, and vopratelimab.

In some embodiments, the chemotherapy is selected from the group consisting of 5-FU, leucovorin, oxaliplatin, irinotecan, gemcitabine, nab-paclitaxel (Abraxane®), FOLFIRINOX, and combinations thereof. In some embodiments, therapeutic agents used to treat pancreatic cancer comprise a combination of (a) 5-FU+leucovorin+oxaliplatin+irinotecan, 5-FU+nanoliposomal irinotecan, leucovorin+nanoliposomal irinotecan, and gemcitabine+nab-paclitaxel.

In some embodiments, the method further comprises co-administering an additional therapeutic agent or therapeutic modality.

### BRIEF DESCRIPTION OF THE DRAWINGS

FIG. 1 shows immunoprecipitation of integrin αV and β1 subunits in various human CAGs.

FIGS. 2A, 2B, and 2C show growth curves of tumors from LSL-Kras^{G12D/+} p53^{R172H/+} PDX-1Cre mice surgically implanted into C57BL/6 female mice. Different mice populations were treated with an isotype control antibody (RatIgG2aK isotype control) and vehicle control BID (Group 1, FIG. 2A), or an anti-PD1 antibody and a vehicle control BID (Group 2, FIG. 2B), or a combination of anti-PD1 antibody and αVβ1/6 inhibitor (bexotegrast) (Group 3, FIG. 2C).

FIG. 3 shows the chemical structure of bexotegrast.

### DETAILED DESCRIPTION

Provided herein are combination therapies for treating cancer in a subject, comprising co-administering to the subject an effective amount of a) an integrin activity modulator; and b) a PD-1 inhibitor or PD-L1 inhibitor. In some embodiments, the PD-1 inhibitor or PD-L1 inhibitor is an anti-PD-1 antibody. In some embodiments, the PD-1 inhibitor or PD-L1 inhibitor is an anti-PD-L1 antibody. In some embodiments, the subject is human. In some embodiments the cancer is pancreatic cancer. In some embodiments the cancer is pancreatic ductal adenocarcinoma (PDAC).

This disclosure is based, at least in part, on the realization that primary human cancer associated fibroblasts (CAFs) from breast cancer and PDAC donors express integrin αV and β1 subunits (see, e.g., Example 1). This disclosure is further based, at least in part, on the realization that combining an integrin activity modulator with a PD-1 inhibitor or a PD-L1 inhibitor (e.g., an anti-PD-1 antibody or anti-PD-L1 antibody), can enhance the anticancer activity of an anti-PD-1 antibody single agent treatment (see, e.g., Example 2), for example in connection with a pancreatic cancer (e.g., PDAC) treatment.

### Definitions

The description below is made with the understanding that the present disclosure is to be considered as an exemplification of the claimed subject matter and is not intended to limit the appended claims to the specific embodiments illustrated. The headings used throughout this disclosure are provided for convenience and are not to be construed to limit the claims in any way. Embodiments illustrated under any heading may be combined with embodiments illustrated under any other heading.

Unless defined otherwise, all technical and scientific terms used herein have the same meaning as commonly understood by one of ordinary skill in the art. It must be noted that as used herein and in the appended claims, the singular forms “a”, “and”, and “the” include plural referents unless the context clearly dictates otherwise. Thus, e.g., reference to “the compound” includes a plurality of such compounds and reference to “the assay” includes reference to one or more assays and equivalents thereof known to those skilled in the art, and so forth.

As used in the present specification, the following terms and phrases are generally intended to have the meanings as set forth below, except to the extent that the context in which they are used indicates otherwise.

Reference to “about” a value or parameter herein includes (and describes) embodiments that are directed to that value or parameter per se. In certain embodiments, the term “about” includes the indicated amount±10%. In other embodiments, the term “about” includes the indicated amount±5%. In certain other embodiments, the term “about” includes the indicated amount±%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

The terms “optional” or “optionally” mean that the subsequently described event or circumstance may or may not occur, and that the description includes instances where said event or circumstance occurs and instances in which it does not.

It will be appreciated by the skilled person that when lists of alternative substituents include members which, because of their valency requirements or other reasons, cannot be used to substitute a particular group, the list is intended to be read with the knowledge of the skilled person to include only those members of the list which are suitable for substituting the particular group.

Further the compounds of the present disclosure may be present in the form of solvates, such as those which include as solvate water, or pharmaceutically acceptable solvates, such as alcohols, in particular ethanol. A “solvate” is formed by the interaction of a solvent and a compound.

Furthermore, the present disclosure provides pharmaceutical compositions comprising a compound of the present disclosure, or a prodrug compound thereof, or a pharmaceutically acceptable salt or solvate thereof as active ingredient together with a pharmaceutically acceptable carrier.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure can encompass any composition made by admixing at least one compound of the present disclosure and a pharmaceutically acceptable carrier.

As used herein, “pharmaceutically acceptable carrier” includes excipients or agents such as solvents, diluents, dispersion media, coatings, antibacterial and antifungal agents, isotonic and absorption delaying agents and the like that are not deleterious to the disclosed compound or use thereof. The use of such carriers and agents to prepare compositions of pharmaceutically active substances is well known in the art (see, e.g., Remington's Pharmaceutical Sciences, Mace Publishing Co., Philadelphia, PA 17th Ed. (1985); and Modern Pharmaceutics, Marcel Dekker, Inc. 3rd Ed. (G. S. Banker & C. T. Rhodes, Eds.).

“IC₅₀” or “EC₅₀” refers to the inhibitory concentration required to achieve 50% of the maximum desired effect. In many cases here the maximum desired effect is the inhibition of LPA induced LPAR1 activation. This term is obtained using an in vitro assay, such as a calcium mobilization assay, evaluating the concentration-dependent inhibition of LPA induced LPAR1 activity.

As used herein, the term “antibody” refers to a polypeptide that includes canonical immunoglobulin sequence elements sufficient to confer specific binding to a particular target antigen (e.g., a heavy chain variable domain, a light chain variable domain, and/or one or more CDRs sufficient to confer specific binding to a particular target antigen). Thus, the term antibody includes, for example, and without limitation, human antibodies, non-human antibodies, antibody fragments, and antigen-binding agents that include antibody fragments, inclusive of synthetic, engineered, and modified forms thereof. The term antibody includes, by way of example, both naturally occurring and non-naturally occurring antibodies. In general, an antibody may comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system. Naturally-produced antibodies are glycosylated, typically on the CH2 domain. Examples of antibodies include monoclonal antibodies, monospecific antibodies, polyclonal antibodies, multispecific antibodies (including bispecific antibodies), engineered antibodies, recombinantly produced antibodies, wholly synthetic antibodies, humanized antibodies, chimeric antibodies, immunoglobulins, tetrameric antibodies comprising two heavy chain and two light chain molecules, antibody light chain monomers, antibody heavy chain monomers, antibody light chain dimers, antibody heavy chain dimers, antibody light chain-antibody heavy chain pairs, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affybodies, Fab fragments, Fab′ fragments, F(ab′)2 fragments, Fd′ fragments, Fd fragments, isolated CDRs, single chain Fvs, polypeptide-Fc fusions, single domain antibodies (e.g., shark single domain antibodies such as IgNAR or fragments thereof); cameloid antibodies; disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), single chain or Tandem diabodies (TandAb®), Anticalins®, Nanobodies®, minibodies, BiTE®s, ankyrin repeat proteins or DARPINs®, Avimers®, DARTs, TCR-like antibodies, Adnectins®, Affilins®, Trans-Bodies®, Affibodies®, TrimerX®, MicroProteins, Fynomers®, Centyrins®, KALBITOR®s, and antigen-binding fragments of any of the above.

As used herein, the term “large molecule” refers to a compound comprising two or more amino acids, such as a peptide, protein fragment, antibody, antibody fragment, or antibody-drug conjugates (ADCs).

As used herein, the term “integrin activity modulator” refers to a compound (e.g., small molecule or large molecule) that activates, inhibits, or interacts with integrin or an integrin subunit. An integrin activity modulator may activate integrin by shifting the integrin from a low affinity to a high affinity ligand-binding state. The shift from low affinity to a high affinity ligand-binding state may result from conformational changes that exposes the ligand-binding site of the integrin. An integrin activity modulator may inhibit the integrin by blocking the ligand-binding site of the integrin or may block one or more integrin subunits (e.g., block the alpha subunit of the integrin or block the beta subunit of the integrin). Alternatively, or additionally, the integrin activity modulator may interact with the integrin or interact with a subunit of the integrin (e.g., interact with the alpha subunit or interact with the beta subunit). In some embodiments, the integrin activity modulator is an integrin-activating agent. As used herein, the term “integrin-activating agent” refers to a compound (e.g., small molecule or large molecule) that (a) activates integrin by shifting the integrin from a low affinity to a high affinity ligand-binding state; (b) increases the activity or function of the integrin; (c) increases the affinity of the integrin to its ligand; or (c) exposes or increases the exposure of the ligand-binding site of the integrin. Alternatively, the integrin activity modulator is an integrin-inhibiting agent. As used herein, the term “integrin-inhibiting agent” refers to a compound (e.g., small molecule or large molecule) that (a) blocks the ligand-binding site of the integrin; (b) reduces the affinity of the integrin for its ligand; (c) shifts the integrin from a high affinity ligand-binding state to a low affinity ligand-binding state; or (d) reduces the activity or function of the integrin.

As used herein, the term “PD-1 inhibitor” refers to a compound (e.g., small molecule or antibody) that a) binds to programmed cell death protein 1 (PD-1, CD279; NCBI Gene ID: 5133); and b) inhibits the PD-1/PD-L1 interaction and PD-1/PD-L1 pathway. As used herein, the term “PD-L1 inhibitor” refers to a compound (e.g., small molecule or antibody) that a) binds to programmed death-ligand 1 (PD-L1, CD274; NCBI Gene ID: 29126); and b) inhibits the PD-1/PD-L1 interaction and PD-1/PD-L1 pathway. The PD-1/PD-L1 pathway and its role in cancer immunotherapy is described, for example, in Salmaninejad et al, J. Cell Physiol (2019) 234 (10): 16824-16837.

As used herein, the terms “effective amount” or “therapeutically effective amount” refer to that amount of a therapeutic agent administered in the methods provided herein (e.g., integrin activity modulator, PD1 inhibitor, PD-L1 inhibitor) that, when administered alone or in combination with another therapeutic agent to a cell, tissue, or subject is sufficient to effect treatment or a beneficial result in the subject. The therapeutically effective amount may vary depending on the subject, and disease or condition being treated, the weight and age of the subject, the severity of the disease or condition, and the manner of administering, which can readily be determined by one of ordinary skill in the art. An effective amount further refers to that amount of the therapeutic agent, which when used in the context of the combination therapies provided herein, is sufficient to treat, prevent, alleviate, ameliorate or mitigate a disease condition, or delay or slow the progression of a disease, and that amount sufficient to effect an increase in rate of treatment, healing, prevention or amelioration of such conditions. When applied to an individual therapeutic agent administered alone, an effective amount refers to that active ingredient alone. When applied to a combination, a therapeutically effective amount refers to combined amounts of the active ingredients that result in the therapeutic effect, whether administered in combination, serially or simultaneously. In some embodiments an effective amount or therapeutically effective amount of a therapeutic agent (e.g., integrin activity modulator, PD1 inhibitor, PD-L1 inhibitor) administered to a subject in the methods provided herein with one or more additional therapeutic agents, as described herein, can (i) reduce the number of diseased cells; (ii) reduce tumor size; (iii) inhibit, retard, slow to some extent, and preferably stop the diseased cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with cancer. In various embodiments, the amount is sufficient to ameliorate, palliate, lessen, and/or delay one or more of symptoms of cancer.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. In some embodiments the methods provided herein refer to the treatment of a subject having cancer (e.g., a human cancer patient). In some embodiments treating a subject having cancer (e.g., a human cancer patient) comprises inhibiting cancer or cancer cell proliferation in the treated subject. In some embodiments treating a human cancer patient using the methods provided herein results in the observation of anti-tumor effects or anti-cancer effects in the treated patient.

As used herein, the terms “inhibition of cancer” and “inhibition of cancer cell proliferation” refer to the inhibition of the growth, division, maturation or viability of cancer cells, and/or causing the death of cancer cells, individually or in aggregate with other cancer cells, by cytotoxicity, nutrient depletion, or the induction of apoptosis.

As used herein, the terms “anti-tumor effect,” “anti-cancer effect,” or “anti-cancer efficacy” refer to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. In some embodiments anti-cancer effects are measured using one or more of the endpoint criteria applied in the clinical studies described herein (e.g., primary, secondary, or exploratory endpoints). Exemplary clinical endpoint criteria that can be used to measure anti-cancer effects in connection with the methods provided herein include objective response rate (ORR), complete response (CR) rate, partial response (PR) rate, disease control rate (DCR), progression-free survival (PFS), duration of response (DOR), overall survival (OS), biomarker-based signals, e.g., of intratumoral immune activation or induction of cancer cell death (e.g., tumor tissue or blood based biomarkers), patient quality of life (QoL) indicators (e.g., based on patient surveys), and others. In some embodiments, anti-tumor effects (e.g., tumor responses or progression) are determined according to RECIST version 1.1 (Eisenhauer et al. Eur J Cancer (2009); 45 (2):228-47). Anti-cancer effects can be observed using any diagnostic methods known to a skilled artisan, such as computed tomography (CT), magnetic resonance imaging (MRI), radiography, or the like.

As used herein, an “increased” or “enhanced” amount is typically a “statistically significant” amount (e.g., with respect to tumor size, cancer cell proliferation or growth), and may include an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. It may also include an increase of at least 10%, at least 20%, at least 30%, at least 40%, at least 50%, at least 60%, at least 70%, at least 80%, at least 90%, at least 100%, at least 150%, at least 200%, at least 500%, or at least 1000% of an amount or level described herein.

As used herein, a “decreased” or “reduced” or “lesser” amount (e.g., with respect to tumor size, cancer cell proliferation or growth) refers to a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein. It may also include a decrease of at least 10%, at least 20%, at least 30%, at least 40%, at least 50%, at least 60%, at least 70%, at least 80%, or at least 90%, at least 100%, at least 150%, at least 200%, at least 500%, or at least 1000% of an amount or level described herein.

### List of Abbreviations and Acronyms

| | |
|---|---|
| BRAC | Breast cancer |
| CAF | Cancer associated fibroblast |
| CPI | Checkpoint Inhibitor |
| CTLA4 | Cytotoxic T-lymphocyte antigen-4 |
| CYP | Cytochrome P450 |
| DNA | Deoxyribonucleic acid |
| EC₅₀ | Half maximal effective concentration |
| FDA | Food and Drug Administration |
| IC₅₀ | Half maximal inhibitory concentration |
| mg | Microgram |
| mg | Milligram |
| mL | Milliliter |
| mTNBC | Metastatic triple-negative breast cancer |
| NA | Not applicable |
| NK | Natural killer (cell) |
| NSCLC | Non-small cell lung cancer |
| PD-1 | Programmed cell death protein 1 |
| PD-L1 | Programmed cell death-ligand 1 |
| PDAC | Pancreatic ductal adenocarcinoma |
| PSA | Prostate-specific antigen |
| QD | Once daily |
| Q3W | Every three weeks |
| RNA | Ribonucleic acid |
| TNBC | Triple-negative breast cancer |

In one aspect, provided herein is a method of treating cancer in a subject, comprising co-administering to the subject an effective amount of a) an integrin activity modulator; and b) a PD-1 inhibitor or PD-L1 inhibitor.

In another aspect, provided herein is an integrin activity modulator for use in combination with a PD-1 inhibitor or a PD-L1 inhibitor (e.g., anti-PD1 antibody or anti-PD-L1 antibody) in a method of treating cancer, wherein the method comprises administering the integrin activity modulator, and PD-1 inhibitor or PD-L1 inhibitor (e.g., anti-PD1 antibody or anti-PD-L1 antibody) to a subject having cancer (e.g., PDAC or BRAC).

In another aspect, provided herein is a PD-1 inhibitor or a PD-L1 inhibitor (e.g., anti-PD1 antibody or anti-PD-L1 antibody) for use in combination with an integrin activity modulator in a method of treating cancer, wherein the method comprises administering the integrin activity modulator, and PD-1 inhibitor or PD-L1 inhibitor (e.g., anti-PD1 antibody or anti-PD-L1 antibody) to a subject having cancer (e.g., PDAC or BRAC).

In some embodiments, the cancer comprises a solid tumor. In some embodiments, the solid tumor is located in or arising from a tissue or organ selected from the group consisting of:
bone (e.g., adamantinoma, aneurysmal bone cysts, angiosarcoma, chondroblastoma, chondroma, chondromyxoid fibroma, chondrosarcoma, chordoma, dedifferentiated chondrosarcoma, enchondroma, epithelioid hemangioendothelioma, fibrous dysplasia of the bone, giant cell tumour of bone, haemangiomas and related lesions, osteoblastoma, osteochondroma, osteosarcoma, osteoid osteoma, osteoma, periosteal chondroma, Desmoid tumor, Ewing sarcoma);
lips and oral cavity (e.g., odontogenic ameloblastoma, oral leukoplakia, oral squamous cell carcinoma, primary oral mucosal melanoma); salivary glands (e.g., pleomorphic salivary gland adenoma, salivary gland adenoid cystic carcinoma, salivary gland mucoepidermoid carcinoma, salivary gland Warthin's tumors);
esophagus (e.g., Barrett's esophagus, dysplasia and adenocarcinoma);
gastrointestinal tract, including stomach (e.g., gastric adenocarcinoma, primary gastric lymphoma, gastrointestinal stromal tumors (GISTs), metastatic deposits, gastric carcinoids, gastric sarcomas, neuroendocrine carcinoma, gastric primary squamous cell carcinoma, gastric adenoacanthomas), intestines and smooth muscle (e.g., intravenous leiomyomatosis), colon (e.g., colorectal adenocarcinoma), rectum, anus;
pancreas (e.g., serous neoplasms, including microcystic or macrocystic serous cystadenoma, solid serous cystadenoma, Von Hippel-Landau (VHL)-associated serous cystic neoplasm, serous cystadenocarcinoma; mucinous cystic neoplasms (MCN), intraductal papillary mucinous neoplasms (IPMN), intraductal oncocytic papillary neoplasms (IOPN), intraductal tubular neoplasms, cystic acinar neoplasms, including acinar cell cystadenoma, acinar cell cystadenocarcinoma, pancreatic adenocarcinoma, invasive pancreatic ductal adenocarcinomas, including tubular adenocarcinoma, adenosquamous carcinoma, colloid carcinoma, medullary carcinoma, hepatoid carcinoma, signet ring cell carcinoma, undifferentiated carcinoma, undifferentiated carcinoma with osteoclast-like giant cells, acinar cell carcinoma, neuroendocrine neoplasms, neuroendocrine microadenoma, neuroendocrine tumors (NET), neuroendocrine carcinoma (NEC), including small cell or large cell NEC, insulinoma, gastrinoma, glucagonoma, serotonin-producing NET, somatostatinoma, VIPoma, solid-pseudopapillary neoplasms (SPN), pancreatoblastoma);
gall bladder (e.g., carcinoma of the gallbladder and extrahepatic bile ducts, intrahepatic cholangiocarcinoma);
neuro-endocrine (e.g., adrenal cortical carcinoma, carcinoid tumors, phaeochromocytoma, pituitary adenomas);
thyroid (e.g., anaplastic (undifferentiated) carcinoma, medullary carcinoma, oncocytic tumors, papillary carcinoma, adenocarcinoma);
liver (e.g., adenoma, combined hepatocellular and cholangiocarcinoma, fibrolamellar carcinoma, hepatoblastoma, hepatocellular carcinoma, mesenchymal, nested stromal epithelial tumor, undifferentiated carcinoma; hepatocellular carcinoma, intrahepatic cholangiocarcinoma, bile duct cystadenocarcinoma, epithelioid hemangioendothelioma, angiosarcoma, embryonal sarcoma, rhabdomyosarcoma, solitary fibrous tumor, teratoma, York sac tumor, carcinosarcoma, rhabdoid tumor);
kidney (e.g., ALK-rearranged renal cell carcinoma, chromophobe renal cell carcinoma, clear cell renal cell carcinoma, clear cell sarcoma, metanephric adenoma, metanephric adenofibroma, mucinous tubular and spindle cell carcinoma, nephroma, nephroblastoma (Wilms tumor), papillary adenoma, papillary renal cell carcinoma, renal oncocytoma, renal cell carcinoma, succinate dehydrogenase-deficient renal cell carcinoma, collecting duct carcinoma);
breast (e.g., invasive ductal carcinoma, including without limitation, acinic cell carcinoma, adenoid cystic carcinoma, apocrine carcinoma, cribriform carcinoma, glycogen-rich/clear cell, inflammatory carcinoma, lipid-rich carcinoma, medullary carcinoma, metaplastic carcinoma, micropapillary carcinoma, mucinous carcinoma, neuroendocrine carcinoma, oncocytic carcinoma, papillary carcinoma, sebaceous carcinoma, secretory breast carcinoma, tubular carcinoma; lobular carcinoma, including without limitation, pleomorphic carcinoma, signet ring cell carcinoma);
peritoneum (e.g., mesothelioma; primary peritoneal cancer);
female sex organ tissues, including ovary (e.g., choriocarcinoma, epithelial tumors, germ cell tumors, sex cord-stromal tumors), Fallopian tubes (e.g., serous adenocarcinoma, mucinous adenocarcinoma, endometrioid adenocarcinoma, clear cell adenocarcinoma, transitional cell carcinoma, squamous cell carcinoma, undifferentiated carcinoma, Müllerian tumors, adenosarcoma, leiomyosarcoma, teratoma, germ cell tumors, choriocarcinoma, trophoblastic tumors), uterus (e.g., carcinoma of the cervix, endometrial polyps, endometrial hyperplasia, intraepithelial carcinoma (EIC), endometrial carcinoma (e.g., endometrioid carcinoma, serous carcinoma, clear cell carcinoma, mucinous carcinoma, squamous cell carcinoma, transitional carcinoma, small cell carcinoma, undifferentiated carcinoma, mesenchymal neoplasia), leiomyoma (e.g., endometrial stromal nodule, leiomyosarcoma, endometrial stromal sarcoma (ESS), mesenchymal tumors), mixed epithelial and mesenchymal tumors (e.g., adenofibroma, carcinofibroma, adenosarcoma, carcinosarcoma (malignant mixed mesodermal sarcoma—MMMT)), endometrial stromal tumors, endometrial malignant mullerian mixed tumours, gestational trophoblastic tumors (partial hydatiform mole, complete hydatiform mole, invasive hydatiform mole, placental site tumour)), vulva, vagina;
male sex organ tissues, including prostate, testis (e.g., germ cell tumors, spermatocytic seminoma), penis;
bladder (e.g., squamous cell carcinoma, urothelial carcinoma, bladder urothelial carcinoma);
brain, (e.g., gliomas (e.g., astrocytomas, including non-infiltrating, low-grade, anaplastic, glioblastomas; oligodendrogliomas, ependymomas), meningiomas, gangliogliomas, schwannomas (neurilemmomas), craniopharyngiomas, chordomas, Non-Hodgkin lymphomas (NHLs), indolent non-Hodgkin's lymphoma (iNHL), refractory iNHL, pituitary tumors;
eye (e.g., retinoma, retinoblastoma, ocular melanoma, posterior uveal melanoma, iris hamartoma);
head and neck (e.g., nasopharyngeal carcinoma, Endolymphatic Sac Tumor (ELST), epidermoid carcinoma, laryngeal cancers including squamous cell carcinoma (SCC) (e.g., glottic carcinoma, supraglottic carcinoma, subglottic carcinoma, transglottic carcinoma), carcinoma in situ, verrucous, spindle cell and basaloid SCC, undifferentiated carcinoma, laryngeal adenocarcinoma, adenoid cystic carcinoma, neuroendocrine carcinomas, laryngeal sarcoma), head and neck paragangliomas (e.g., carotid body, jugulotympanic, vagal);
thymus (e.g., thymoma);
heart (e.g., cardiac myxoma);
lung (e.g., small cell carcinoma (SCLC), non-small cell lung carcinoma (NSCLC), including squamous cell carcinoma (SCC), adenocarcinoma and large cell carcinoma, carcinoids (typical or atypical), carcinosarcomas, pulmonary blastomas, giant cell carcinomas, spindle cell carcinomas, pleuropulmonary blastoma);
lymph (e.g., lymphomas, including Hodgkin's lymphoma, non-Hodgkin's lymphoma (NHL), indolent non-Hodgkin's lymphoma (iNHL), refractory iNHL, Epstein-Barr virus (EBV)-associated lymphoproliferative diseases, including B cell lymphomas and T cell lymphomas (e.g., Burkitt lymphoma; large B cell lymphoma, diffuse large B-cell lymphoma (DLBCL), mantle cell lymphoma, indolent B-cell lymphoma, low grade B cell lymphoma, fibrin-associated diffuse large cell lymphoma; primary effusion lymphoma; plasmablastic lymphoma; extranodal NK/T cell lymphoma, nasal type; peripheral T cell lymphoma, cutaneous T cell lymphoma, angioimmunoblastic T cell lymphoma; follicular T cell lymphoma; systemic T cell lymphoma), lymphangioleiomyomatosis);
central nervous system (CNS) (e.g., gliomas including astrocytic tumors (e.g., pilocytic astrocytoma, pilomyxoid astrocytoma, subependymal giant cell astrocytoma, pleomorphic xanthoastrocytoma, diffuse astrocytoma, fibrillary astrocytoma, gemistocytic astrocytoma, protoplasmic astrocytoma, anaplastic astrocytoma, glioblastoma (e.g., giant cell glioblastoma, gliosarcoma, glioblastoma multiforme) and gliomatosis cerebri), oligodendroglial tumors (e.g., oligodendroglioma, anaplastic oligodendroglioma), oligoastrocytic tumors (e.g., oligoastrocytoma, anaplastic oligoastrocytoma), ependymal tumors (e.g., subependymom, myxopapillary ependymoma, ependymomas (e.g., cellular, papillary, clear cell, tanycytic), anaplastic ependymoma), optic nerve glioma, and non-gliomas (e.g., choroid plexus tumors, neuronal and mixed neuronal-glial tumors, pineal region tumors, embryonal tumors, medulloblastoma, meningeal tumors, primary CNS lymphomas, germ cell tumors, Pituitary adenomas, cranial and paraspinal nerve tumors, stellar region tumors); neurofibroma, meningioma, peripheral nerve sheath tumors, peripheral neuroblastic tumours (including without limitation neuroblastoma, ganglioneuroblastoma, ganglioneuroma), trisomy 19 ependymoma);
neuroendocrine tissues (e.g., paraganglionic system including adrenal medulla (pheochromocytomas) and extra-adrenal paraganglia ((extra-adrenal) paragangliomas);
skin (e.g., clear cell hidradenoma, cutaneous benign fibrous histiocytomas, cylindroma, hidradenoma, melanoma (including cutaneous melanoma, mucosal melanoma), pilomatricoma, Spitz tumors); and
soft tissues (e.g., aggressive angiomyxoma, alveolar rhabdomyosarcoma, alveolar soft part sarcoma, angiofibroma, angiomatoid fibrous histiocytoma, synovial sarcoma, biphasic synovial sarcoma, clear cell sarcoma, dermatofibrosarcoma protuberans, desmoid-type fibromatosis, small round cell tumor, desmoplastic small round cell tumor, elastofibroma, embryonal rhabdomyosarcoma, Ewing's tumors/primitive neurectodermal tumors (PNET), extraskeletal myxoid chondrosarcoma, extraskeletal osteosarcoma, paraspinal sarcoma, inflammatory myofibroblastic tumor, lipoblastoma, lipoma, chondroid lipoma, liposarcoma/malignant lipomatous tumors, liposarcoma, myxoid liposarcoma, fibromyxoid sarcoma, lymphangioleiomyoma, malignant myoepithelioma, malignant melanoma of soft parts, myoepithelial carcinoma, myoepithelioma, myxoinflammatory fibroblastic sarcoma, undifferentiated sarcoma, pericytoma, rhabdomyosarcoma, non-rhabdomyosarcoma soft tissue sarcoma (NRSTS), soft tissue leiomyosarcoma, undifferentiated sarcoma, well-differentiated liposarcoma.

In some embodiments, the cancer is selected from breast cancer or pancreatic cancer. In some embodiments, the cancer is breast cancer. In some embodiments, the breast cancer is triple-negative breast cancer (TNBC), HR+/HER2− breast cancer (HR+/HER2− BC), HR+/HER2low breast cancer (HR+/HER2low BC). In some embodiments, the cancer is pancreatic cancer. In some embodiments the pancreatic cancer is pancreatic ductal adenocarcinoma (PDAC).

In some embodiments, the cancer is metastatic.

In some embodiments, the subject is human.

In some embodiments, the subject is treatment naïve.

In some embodiments, the subject has received at least one previous anticancer treatment selected from the group consisting of surgery, radiation therapy, chemotherapy, or checkpoint inhibitor therapy. In some embodiments, the cancer has progressed or recurred following an anticancer treatment selected from the group consisting of surgery, radiation therapy, chemotherapy, or checkpoint inhibitor therapy. In some embodiments, the cancer is resistant or refractory to checkpoint inhibitor therapy. In some embodiments, the checkpoint inhibitor therapy comprises an anti-PD-1 antibody or anti-PD-L1 antibody. In some embodiments, the anti-PD-1 antibody or anti-PD-L1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), MGD-019 (PD-1/CTLA4), KN-046 (PD-1/CTLA4), XmAb-20717 (PD-1/CTLA4), AK-104 (CTLA4/PD-1), MEDI-5752 (CTLA4/PD-1), atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, atezolizumab, avelumab, envafolimab, durvalumab, cosibelimab, lodapolimab, garivulimab, envafolimab, opucolimab, and manelimab. In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, and vopratelimab. In some embodiments, the chemotherapy is selected from the group consisting of 5-FU, leucovorin, oxaliplatin, irinotecan, gemcitabine, nab-paclitaxel (Abraxane®), FOLFIRINOX, and combinations thereof. In some embodiments therapeutic agents used to treat pancreatic cancer include 5-FU+leucovorin+oxaliplatin+irinotecan, (b) 5-FU+nanoliposomal irinotecan, (c) leucovorin+nanoliposomal irinotecan, or (d) gemcitabine+nab-paclitaxel.

In some embodiments, the methods provided herein further comprise co-administering an additional therapeutic agent or therapeutic modality.

### Integrin Inhibitors

The integrin activity modulators that can be co-administered in the methods provided herein can include small molecule or large molecule modulators of integrin activity.

In some embodiments, the integrin activity modulator is selected from an integrin-activating agent, an integrin-inhibiting agent, or an agent that is capable of interacting with one or more integrins.

In some embodiments, the integrin activity modulator is a small molecule or a large molecule. In some embodiments, the large molecule is an antibody or antibody fragment. In some embodiments, the integrin activity modulator is selected from an anti-integrin antibody disclosed in WO2020210358. In some embodiments, the large molecule is an inhibitory peptide or peptide antagonist.

In some embodiments, the integrin activity modulator activates, inhibits, or interacts with an integrin alpha (α) subunit. In some embodiments, the integrin a subunit is selected from α5, αV, α8, and αIIb. In some embodiments, the integrin activity modulator activates, inhibits, or interacts with the αV subunit. In some embodiments, the integrin activity modulator is an anti-αV antibody. In some embodiments, the anti-αV antibody is selected from abituzumab and intetumumab.

In some embodiments, the integrin activity modulator is an iRGD peptide. In some embodiments, the iRGD peptide is CEND-1.

In some embodiments, the integrin activity modulator activates, inhibits, or interacts with an integrin beta (β) subunit. In some embodiments, the β subunit is selected from β1, β3, β5, and β8. In some embodiments, the integrin activity modulator activates, inhibits, or interacts with 1. In some embodiments, the integrin activity modulator is an anti-β1 antibody. In some embodiments, the anti-β1 antibody is OS2966.

In some embodiments, the integrin activity modulator activates, inhibits, or interacts with β5. In some embodiments, the integrin activity modulator is an anti-β5 antibody. In some embodiments, the anti-β5 antibody is ALULA.

In some embodiments, the integrin activity modulator activates, inhibits, or interacts with an RGD-binding integrin. In some embodiments, RGD-binding integrin is selected from α5β1, αVβ1, αVβ3, αVβ5, αVβ6, αVβ8, α8β1, and αIIbβ3.

In some embodiments, the integrin activity modulator activates, inhibits, or interacts with α5β1. In some embodiments, the integrin activity modulator is selected from AG-73305, AP25, AMEP, ATN-161, F-200 (Eos), JSM-6427, liposomal contortrostatin, PF-04605412, bexotegrast (i.e., PLN-74809), and SJ-749.

In some embodiments, the integrin activity modulator activates, inhibits, or interacts with αVβ1. In some embodiments, the integrin activity modulator is selected from bexotegrast (i.e., PLN-74809), IDL-2965, PLN-1474, PLN-1561, and volociximab. In some embodiments, the integrin activity modulator is selected from bexotegrast (i.e., PLN-74809), PLN-1474, and PLN-1561. In some embodiments, the integrin activity modulator is bexotegrast. In some embodiments, the integrin activity modulator is PLN-1474. In some embodiments, the integrin activity modulator is PLN-1561.

In some embodiments, the integrin activity modulator activates, inhibits, or interacts with αVβ3. In some embodiments, the integrin activity modulator is selected from AP25, B-1451, BS-1417, cilengitide, CT-6725, efatucizumab, etaracizumab, FF-10158, HM-3, IPS-02001, IPS-02101, IPS-05002, JNJ-26076713, MK-0429, NTA-1, NVX-188, Ocurin, ProAgio, PS-388023, RP-697, RP-748, SB-273005, Sch-221153, SC-69000, SD-983, SF-0166, SM-256, SOM-0777, SQ-885, TSRI-265, VIP-236, vitaxin, VPI-2690B, and XT-199.

In some embodiments, the integrin activity modulator activates, inhibits, or interacts with αVβ5. In some embodiments, the integrin activity modulator is selected from ALULA, SNAKA-51, and STX-200.

In some embodiments, the integrin activity modulator activates, inhibits, or interacts with αVβ6. In some embodiments, the integrin activity modulator is selected from 264-RAD, A20FMDV2, abituzumab, ADVCA0848, bexotegrast (i.e., PLN-74809), CRB-602, GSK-3008348, IDL-2965, intetumumab, MORF-627, MORF-720, PLN-1177, PLN-1561, PLN-1705, SGN-B6A, and STX-100.

In some embodiments, the integrin activity modulator activates, inhibits, or interacts with αVβ8. In some embodiments, the integrin activity modulator is selected from CRB-601 and PF-06940434.

In some embodiments, the integrin activity modulator activates, inhibits, or interacts with α8β1. In some embodiments, the integrin activity modulator is an anti-α8β1 antibody. In some embodiments, the anti-α8β1 antibody is an anti-α8β1 antibody disclosed in WO2011049082.

In some embodiments, the integrin activity modulator activates, inhibits, or interacts with αIIbβ3. In some embodiments, the integrin activity modulator is selected from eptifibatide, RUC-1, Tc-99m-P280, tirofiban, zalunfiban, and an antibody that interacts with αIIbβ3.

In some embodiments, the methods and kits disclosed herein comprise any one of the integrin activity modulators found, disclosed, or described in any one of Bergonzini, et al., Front Cell Dev Biol, 2022 Mar. 9, 10:863850. doi: 10.3389/fcell.2022.863850. eCollection 2022, Li, et al., Acta Pharm Sin B. 2021 September; 11(9):2726-2737. doi: 10.1016/j.apsb.2021.01.004, Raab-Westphal, et al., Cancers (Basel). 2017 Aug. 23; 9(9):110. doi: 10.3390/cancers9090110, WO2001053262, WO2002031142, WO2001080872, WO2003075957, WO2003087340, WO2009026681, WO2011146819, WO2013123152, WO2017132620, WO2017218569, US20160009806, US20160368993, US20160376368, US20190263913, US20200031938, US20200121788, and US20210340260.

### PD-1 Inhibitors and PD-L1 Inhibitors

The PD-1 inhibitors or PD-L1 inhibitors that can be co-administered in the methods provided herein can include small molecule or large molecule inhibitors of PD-1 or PD-L1.

In some embodiments of the methods provided herein the PD-1 inhibitor or PD-L1 inhibitor that can be co-administered is an anti-PD-1 antibody. In some embodiments, the anti-PD-1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), MGD-019 (PD-1/CTLA4), KN-046 (PD-1/CTLA4), XmAb-20717 (PD-1/CTLA4), AK-104 (CTLA4/PD-1) and MEDI-5752 (CTLA4/PD-1). In some embodiments, the anti-PD-1 antibody is zimberelimab. The sequence for zimberelimab is disclosed as CAS registry number 2259860-24-5 or KEGG entry D12063 or as SEQ ID NO: 53 (heavy chain) and SEQ ID NO: 67 (light chain) of International Publication No. WO2017/025051.

In some embodiments of the methods provided herein, the PD-1 inhibitor or PD-L1 inhibitor that can be co-administered is an anti-PD-L1 antibody. In some embodiments, the anti-PD-L1 antibody is selected from the group consisting of atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR).

In some embodiments of the methods provided herein, the PD-1 inhibitor or PD-L1 inhibitor that can be co-administered is a small molecule PD-L1 inhibitor. In some embodiments, the small molecule PD-L1 inhibitor is selected from the group consisting of CA-170, GS-4224, GS-4416, INCB99280, INCB99318, and lazertinib.

In some embodiments, the methods and kits disclosed herein comprise any one of the PD-1 or PD-L1 inhibitors found, disclosed, or described in any one of Ettl, et al., Cancers (Basel). 2022 Oct. 11; 14(20):4985. doi: 10.3390/cancers14204985, Singh, et al., Biosensors (Basel). 2022 Aug. 8; 12(8):617. doi: 10.3390/bios12080617, Wang, et al., J Hematol Oncol. 2022 Aug. 17; 15(1):111. doi: 10.1186/s13045-022-01325-0, WO2016137985, WO2016160792, WO2016191751, WO2014022758, WO2016090300, WO2018150224, WO2019032663, WO2019161129, WO2020223217, WO2021036929, WO2021097800, WO2021177980, WO2021194481, WO2022050954, WO2022089377, WO2022115719, US20100086550, US20200339654, and US20220135686.

### Kits

Disclosed herein are kits for use as a medicament, wherein the kit comprises (a) an integrin activity modulator; and (b) a PD-1 inhibitor or PD-L1 inhibitor. In some embodiments, the integrin activity modulator is selected from an integrin-activating agent, an integrin-inhibiting agent, or an agent that is capable of interacting with one or more integrins. In some embodiments, the integrin activity modulator is a small molecule or a large molecule. In some embodiments, the large molecule is an antibody or antibody fragment. In some embodiments, the large molecule is an inhibitory peptide or peptide antagonist. In some embodiments, the integrin activity modulator activates, inhibits, or interacts with an integrin alpha (α) subunit. In some embodiments, the integrin a subunit is selected from α5, αV, α8, and αIIb. In some embodiments, the integrin activity modulator activates, inhibits, or interacts with the αV subunit. In some embodiments, the integrin activity modulator is an anti-αV antibody. In some embodiments, the anti-αV antibody is selected from abituzumab and intetumumab. In some embodiments, the integrin activity modulator is an iRGD peptide. In some embodiments, the iRGD peptide is CEND-1. In some embodiments, the integrin activity modulator activates, inhibits, or interacts with an integrin beta (β) subunit. In some embodiments, the β subunit is selected from β1, β3, β5, and β8. In some embodiments, the integrin activity modulator activates, inhibits, or interacts with β1. In some embodiments, the integrin activity modulator is an anti-β1 antibody. In some embodiments, the anti-β1 antibody is OS2966. In some embodiments, the integrin activity modulator activates, inhibits, or interacts with β5. In some embodiments, the integrin activity modulator is an anti-β5 antibody. In some embodiments, the anti-β5 antibody is ALULA. In some embodiments, the integrin activity modulator activates, inhibits, or interacts with an RGD-binding integrin. In some embodiments, RGD-binding integrin is selected from α5β1, αVβ1, αVβ3, αVβ5, αVβ6, αVβ8, α8β1, and αIIbβ3. In some embodiments, the integrin activity modulator activates, inhibits, or interacts with α5β1. In some embodiments, the integrin activity modulator is selected from AG-73305, AP25, AMEP, ATN-161, F-200 (Eos), JSM-6427, liposomal contortrostatin, PF-04605412, bexotegrast (i.e., PLN-74809), and SJ-749. In some embodiments, the integrin activity modulator activates, inhibits, or interacts with αVβ1. In some embodiments, the integrin activity modulator is selected from bexotegrast (i.e., PLN-74809), IDL-2965, PLN-1474, PLN-1561, and volociximab. In some embodiments, the integrin activity modulator is selected from bexotegrast (i.e., PLN-74809), PLN-1474, and PLN-1561. In some embodiments, the integrin activity modulator is bexotegrast. In some embodiments, the integrin activity modulator is PLN-1474. In some embodiments, the integrin activity modulator is PLN-1561. In some embodiments, the integrin activity modulator activates, inhibits, or interacts with αVβ3. In some embodiments, the integrin activity modulator is selected from AP25, B-1451, BS-1417, cilengitide, CT-6725, efatucizumab, etaracizumab, FF-10158, HM-3, IPS-02001, IPS-02101, IPS-05002, JNJ-26076713, MK-0429, NTA-1, NVX-188, Ocurin, ProAgio, PS-388023, RP-697, RP-748, SB-273005, Sch-221153, SC-69000, SD-983, SF-0166, SM-256, SOM-0777, SQ-885, TSRI-265, VIP-236, vitaxin, VPI-2690B, and XT-199. In some embodiments, the integrin activity modulator activates, inhibits, or interacts with αVβ5. In some embodiments, the integrin activity modulator is selected from ALULA, SNAKA-51, and STX-200. In some embodiments, the integrin activity modulator activates, inhibits, or interacts with αVβ6. In some embodiments, the integrin activity modulator is selected from 264-RAD, A20FMDV2, abituzumab, ADVCA0848, bexotegrast (i.e., PLN-74809), CRB-602, GSK-3008348, IDL-2965, intetumumab, MORF-627, MORF-720, PLN-1177, PLN-1561, PLN-1705, SGN-B6A, and STX-100. In some embodiments, the integrin activity modulator activates, inhibits, or interacts with αVβ8. In some embodiments, the integrin activity modulator is selected from CRB-601 and PF-06940434. In some embodiments, the integrin activity modulator activates, inhibits, or interacts with α8β1. In some embodiments, the integrin activity modulator is an anti-α8β1 antibody. In some embodiments, the anti-α8β1 antibody is an anti-α8β1 antibody disclosed in WO2011049082. In some embodiments, the integrin activity modulator activates, inhibits, or interacts with αIIbβ3. In some embodiments, the integrin activity modulator is selected from eptifibatide, RUC-1, Tc-99m-P280, tirofiban, zalunfiban, and an antibody that interacts with αIIbβ3. In some embodiments, the PD-1 inhibitor or PD-L1 inhibitor is an anti-PD-1 antibody. In some embodiments, the anti-PD-1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), PF-06801591, MGD-019 (PD-1/CTLA4), KN-046 (PD-1/CTLA4), XmAb-20717 (PD-1/CTLA4), AK-104 (CTLA4/PD-1), and MEDI-5752 (CTLA4/PD-1). In some embodiments, the anti-PD-1 antibody is zimberelimab. In some embodiments, the PD-1 inhibitor or PD-L1 inhibitor is an anti-PD-L1 antibody. In some embodiments, the PD-L1 inhibitor is selected from the group consisting of atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the PD-L1 inhibitor is a small molecule inhibitor. In some embodiments, the small molecule PD-L1 inhibitor is selected from the group consisting of CA-170, GS-4224, GS-4416, INCB99280, INCB99318, and lazertinib. In some embodiments, the PD-1 inhibitor or PD-L1 inhibitor is atezolizumab. In some embodiments, the PD-1 inhibitor or PD-L1 inhibitor is durvalumab. In some embodiments, the PD-1 inhibitor or PD-L1 inhibitor is MK-7684A. In some embodiments, the PD-1 inhibitor or PD-L1 inhibitor is nivolumab. In some embodiments, the PD-1 inhibitor or PD-L1 inhibitor is pembrolizumab. In some embodiments, the PD-1 inhibitor or PD-L1 inhibitor is zimberelimab. In some embodiments, the cancer is a solid tumor. In some embodiments, the cancer is selected from breast cancer or pancreatic cancer. In some embodiments, the cancer is breast cancer. In some embodiments, the breast cancer is triple-negative breast cancer (TNBC), HR+/HER2− breast cancer (HR+/HER2− BC), HR+/HER2low breast cancer (HR+/HER2low BC). In some embodiments, the cancer is pancreatic cancer. In some embodiments, the pancreatic cancer is pancreatic ductal adenocarcinoma (PDAC). In some embodiments, the cancer is metastatic. In some embodiments, the subject is human. In some embodiments, the subject is treatment naïve. In some embodiments, the subject has received at least one previous anticancer treatment selected from the group consisting of surgery, radiation therapy, chemotherapy, or checkpoint inhibitor therapy. In some embodiments, the cancer has progressed or recurred following an anticancer treatment selected from the group consisting of surgery, radiation therapy, chemotherapy, or checkpoint inhibitor therapy. In some embodiments, the cancer is resistant or refractory to checkpoint inhibitor therapy. In some embodiments, the checkpoint inhibitor therapy comprises an anti-PD-1 antibody or anti-PD-L1 antibody. In some embodiments, the anti-PD-1 antibody or anti-PD-L1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), MGD-019 (PD-1/CTLA4), KN-046 (PD-1/CTLA4), XmAb-20717 (PD-1/CTLA4), AK-104 (CTLA4/PD-1), MEDI-5752 (CTLA4/PD-1), atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, atezolizumab, avelumab, envafolimab, durvalumab, cosibelimab, lodapolimab, garivulimab, envafolimab, opucolimab, and manelimab. In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, and vopratelimab. In some embodiments, the chemotherapy is selected from the group consisting of 5-FU, leucovorin, oxaliplatin, irinotecan, gemcitabine, nab-paclitaxel (Abraxane®), FOLFIRINOX, and combinations thereof. In some embodiments, therapeutic agents used to treat pancreatic cancer comprise a combination of (a) 5-FU+leucovorin+oxaliplatin+irinotecan, (b) 5-FU+nanoliposomal irinotecan, (c) leucovorin+nanoliposomal irinotecan, or (d) gemcitabine+nab-paclitaxel. In some embodiments, the kit further comprises an additional therapeutic agent or therapeutic modality.

Further disclosed herein are kits for use in the treatment, mitigation, reduction, prevention, or delay of the recurrence or metastasis of cancer, wherein the kit comprises (a) an integrin activity modulator; and (b) a PD-1 inhibitor or PD-L1 inhibitor. In some embodiments, the integrin activity modulator is selected from an integrin-activating agent, an integrin-inhibiting agent, or an agent that is capable of interacting with one or more integrins. In some embodiments, the integrin activity modulator is a small molecule or a large molecule. In some embodiments, the large molecule is an antibody or antibody fragment. In some embodiments, the large molecule is an inhibitory peptide or peptide antagonist. In some embodiments, the integrin activity modulator activates, inhibits, or interacts with an integrin alpha (α) subunit. In some embodiments, the integrin a subunit is selected from α5, αV, α8, and αIIb. In some embodiments, the integrin activity modulator activates, inhibits, or interacts with the αV subunit. In some embodiments, the integrin activity modulator is an anti-αV antibody. In some embodiments, the anti-αV antibody is selected from abituzumab and intetumumab. In some embodiments, the integrin activity modulator is an iRGD peptide. In some embodiments, the iRGD peptide is CEND-1. In some embodiments, the integrin activity modulator activates, inhibits, or interacts with an integrin beta (β) subunit. In some embodiments, the β subunit is selected from β1, β3, β5, and β8. In some embodiments, the integrin activity modulator activates, inhibits, or interacts with β1. In some embodiments, the integrin activity modulator is an anti-β1 antibody. In some embodiments, the anti-β1 antibody is OS2966. In some embodiments, the integrin activity modulator activates, inhibits, or interacts with β5. In some embodiments, the integrin activity modulator is an anti-β5 antibody. In some embodiments, the anti-β5 antibody is ALULA. In some embodiments, the integrin activity modulator activates, inhibits, or interacts with an RGD-binding integrin. In some embodiments, RGD-binding integrin is selected from α5β1, αVβ1, αVβ3, αVβ5, αVβ6, αVβ8, α8β1, and αIIbβ3. In some embodiments, the integrin activity modulator activates, inhibits, or interacts with α5β1. In some embodiments, the integrin activity modulator is selected from AG-73305, AP25, AMEP, ATN-161, F-200 (Eos), JSM-6427, liposomal contortrostatin, PF-04605412, bexotegrast (i.e., PLN-74809), and SJ-749. In some embodiments, the integrin activity modulator activates, inhibits, or interacts with αVβ1. In some embodiments, the integrin activity modulator is selected from bexotegrast (i.e., PLN-74809), IDL-2965, PLN-1474, PLN-1561, and volociximab. In some embodiments, the integrin activity modulator is selected from bexotegrast (i.e., PLN-74809), PLN-1474, and PLN-1561. In some embodiments, the integrin activity modulator is bexotegrast. In some embodiments, the integrin activity modulator is PLN-1474. In some embodiments, the integrin activity modulator is PLN-1561. In some embodiments, the integrin activity modulator activates, inhibits, or interacts with αVβ3. In some embodiments, the integrin activity modulator is selected from AP25, B-1451, BS-1417, cilengitide, CT-6725, efatucizumab, etaracizumab, FF-10158, HM-3, IPS-02001, IPS-02101, IPS-05002, JNJ-26076713, MK-0429, NTA-1, NVX-188, Ocurin, ProAgio, PS-388023, RP-697, RP-748, SB-273005, Sch-221153, SC-69000, SD-983, SF-0166, SM-256, SOM-0777, SQ-885, TSRI-265, VIP-236, vitaxin, VPI-2690B, and XT-199. In some embodiments, the integrin activity modulator activates, inhibits, or interacts with αVβ5. In some embodiments, the integrin activity modulator is selected from ALULA, SNAKA-51, and STX-200. In some embodiments, the integrin activity modulator activates, inhibits, or interacts with αVβ6. In some embodiments, the integrin activity modulator is selected from 264-RAD, A20FMDV2, abituzumab, ADVCA0848, bexotegrast (i.e., PLN-74809), CRB-602, GSK-3008348, IDL-2965, intetumumab, MORF-627, MORF-720, PLN-1177, PLN-1561, PLN-1705, SGN-B6A, and STX-100. In some embodiments, the integrin activity modulator activates, inhibits, or interacts with αVβ8. In some embodiments, the integrin activity modulator is selected from CRB-601 and PF-06940434. In some embodiments, the integrin activity modulator activates, inhibits, or interacts with α8β1. In some embodiments, the integrin activity modulator is an anti-α8β1 antibody. In some embodiments, the anti-α8β1 antibody is an anti-α8β1 antibody disclosed in WO2011049082. In some embodiments, the integrin activity modulator activates, inhibits, or interacts with αIIbβ3. In some embodiments, the integrin activity modulator is selected from eptifibatide, RUC-1, Tc-99m-P280, tirofiban, zalunfiban, and an antibody that interacts with αIIbβ3. In some embodiments, the PD-1 inhibitor or PD-L1 inhibitor is an anti-PD-1 antibody. In some embodiments, the anti-PD-1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), PF-06801591, MGD-019 (PD-1/CTLA4), KN-046 (PD-1/CTLA4), XmAb-20717 (PD-1/CTLA4), AK-104 (CTLA4/PD-1), and MEDI-5752 (CTLA4/PD-1). In some embodiments, the anti-PD-1 antibody is zimberelimab. In some embodiments, the PD-1 inhibitor or PD-L1 inhibitor is an anti-PD-L1 antibody. In some embodiments, the PD-L1 inhibitor is selected from the group consisting of atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the PD-L1 inhibitor is a small molecule inhibitor. In some embodiments, the small molecule PD-L1 inhibitor is selected from the group consisting of CA-170, GS-4224, GS-4416, INCB99280, INCB99318, and lazertinib. In some embodiments, the PD-1 inhibitor or PD-L1 inhibitor is atezolizumab. In some embodiments, the PD-1 inhibitor or PD-L1 inhibitor is durvalumab. In some embodiments, the PD-1 inhibitor or PD-L1 inhibitor is MK-7684A. In some embodiments, the PD-1 inhibitor or PD-L1 inhibitor is nivolumab. In some embodiments, the PD-1 inhibitor or PD-L1 inhibitor is pembrolizumab. In some embodiments, the PD-1 inhibitor or PD-L1 inhibitor is zimberelimab. In some embodiments, the cancer is a solid tumor. In some embodiments, the cancer is selected from breast cancer or pancreatic cancer. In some embodiments, the cancer is breast cancer. In some embodiments, the breast cancer is triple-negative breast cancer (TNBC), HR+/HER2− breast cancer (HR+/HER2− BC), HR+/HER2low breast cancer (HR+/HER2low BC). In some embodiments, the cancer is pancreatic cancer. In some embodiments, the pancreatic cancer is pancreatic ductal adenocarcinoma (PDAC). In some embodiments, the cancer is metastatic. In some embodiments, the subject is human. In some embodiments, the subject is treatment naïve. In some embodiments, the subject has received at least one previous anticancer treatment selected from the group consisting of surgery, radiation therapy, chemotherapy, or checkpoint inhibitor therapy. In some embodiments, the cancer has progressed or recurred following an anticancer treatment selected from the group consisting of surgery, radiation therapy, chemotherapy, or checkpoint inhibitor therapy. In some embodiments, the cancer is resistant or refractory to checkpoint inhibitor therapy. In some embodiments, the checkpoint inhibitor therapy comprises an anti-PD-1 antibody or anti-PD-L1 antibody. In some embodiments, the anti-PD-1 antibody or anti-PD-L1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), MGD-019 (PD-1/CTLA4), KN-046 (PD-1/CTLA4), XmAb-20717 (PD-1/CTLA4), AK-104 (CTLA4/PD-1), MEDI-5752 (CTLA4/PD-1), atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, atezolizumab, avelumab, envafolimab, durvalumab, cosibelimab, lodapolimab, garivulimab, envafolimab, opucolimab, and manelimab. In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, and vopratelimab. In some embodiments, the chemotherapy is selected from the group consisting of 5-FU, leucovorin, oxaliplatin, irinotecan, gemcitabine, nab-paclitaxel (Abraxane®), FOLFIRINOX, and combinations thereof. In some embodiments, therapeutic agents used to treat pancreatic cancer comprise a combination of (a) 5-FU+leucovorin+oxaliplatin+irinotecan, (b) 5-FU+nanoliposomal irinotecan, (c) leucovorin+nanoliposomal irinotecan, or (d) gemcitabine+nab-paclitaxel. In some embodiments, the kit further comprises an additional therapeutic agent or therapeutic modality.

Further disclosed herein are kits for use as a medicament, wherein the kit comprises (a) an integrin activity modulator; and (b) a PD-1 inhibitor or PD-L1 inhibitor, wherein (i) the integrin activity modulator is selected from bexotegrast (i.e., PLN-74809), PLN-1474, and PLN-1561; or (ii) the PD-1 inhibitor or PD-L1 inhibitor is selected from atezolizumab, durvalumab, MK-7684A, nivolumab, pembrolizumab, and zimberelimab. In some embodiments, the integrin activity modulator is selected from an integrin-activating agent, an integrin-inhibiting agent, or an agent that is capable of interacting with one or more integrins. In some embodiments, the integrin activity modulator is a small molecule or a large molecule. In some embodiments, the large molecule is an antibody or antibody fragment. In some embodiments, the large molecule is an inhibitory peptide or peptide antagonist. In some embodiments, the integrin activity modulator activates, inhibits, or interacts with an integrin alpha (α) subunit. In some embodiments, the integrin a subunit is selected from α5, αV, α8, and αIIb. In some embodiments, the integrin activity modulator activates, inhibits, or interacts with the αV subunit. In some embodiments, the integrin activity modulator is an anti-αV antibody. In some embodiments, the anti-αV antibody is selected from abituzumab and intetumumab. In some embodiments, the integrin activity modulator is an iRGD peptide. In some embodiments, the iRGD peptide is CEND-1. In some embodiments, the integrin activity modulator activates, inhibits, or interacts with an integrin beta (β) subunit. In some embodiments, the β subunit is selected from β1, β3, β5, and β8. In some embodiments, the integrin activity modulator activates, inhibits, or interacts with β1. In some embodiments, the integrin activity modulator is an anti-β1 antibody. In some embodiments, the anti-β1 antibody is OS2966. In some embodiments, the integrin activity modulator activates, inhibits, or interacts with β5. In some embodiments, the integrin activity modulator is an anti-β5 antibody. In some embodiments, the anti-β5 antibody is ALULA. In some embodiments, the integrin activity modulator activates, inhibits, or interacts with an RGD-binding integrin. In some embodiments, RGD-binding integrin is selected from α5β1, αVβ1, αVβ3, αVβ5, αVβ6, αVβ8, α8β1, and αIIbβ3. In some embodiments, the integrin activity modulator activates, inhibits, or interacts with α5β1. In some embodiments, the integrin activity modulator is selected from AG-73305, AP25, AMEP, ATN-161, F-200 (Eos), JSM-6427, liposomal contortrostatin, PF-04605412, bexotegrast (i.e., PLN-74809), and SJ-749. In some embodiments, the integrin activity modulator activates, inhibits, or interacts with αVβ1. In some embodiments, the integrin activity modulator is selected from bexotegrast (i.e., PLN-74809), IDL-2965, PLN-1474, PLN-1561, and volociximab. In some embodiments, the integrin activity modulator is selected from bexotegrast (i.e., PLN-74809), PLN-1474, and PLN-1561. In some embodiments, the integrin activity modulator is bexotegrast. In some embodiments, the integrin activity modulator is PLN-1474. In some embodiments, the integrin activity modulator is PLN-1561. In some embodiments, the integrin activity modulator activates, inhibits, or interacts with αVβ3. In some embodiments, the integrin activity modulator is selected from AP25, B-1451, BS-1417, cilengitide, CT-6725, efatucizumab, etaracizumab, FF-10158, HM-3, IPS-02001, IPS-02101, IPS-05002, JNJ-26076713, MK-0429, NTA-1, NVX-188, Ocurin, ProAgio, PS-388023, RP-697, RP-748, SB-273005, Sch-221153, SC-69000, SD-983, SF-0166, SM-256, SOM-0777, SQ-885, TSRI-265, VIP-236, vitaxin, VPI-2690B, and XT-199. In some embodiments, the integrin activity modulator activates, inhibits, or interacts with αVβ5. In some embodiments, the integrin activity modulator is selected from ALULA, SNAKA-51, and STX-200. In some embodiments, the integrin activity modulator activates, inhibits, or interacts with αVβ6. In some embodiments, the integrin activity modulator is selected from 264-RAD, A20FMDV2, abituzumab, ADVCA0848, bexotegrast (i.e., PLN-74809), CRB-602, GSK-3008348, IDL-2965, intetumumab, MORF-627, MORF-720, PLN-1177, PLN-1561, PLN-1705, SGN-B6A, and STX-100. In some embodiments, the integrin activity modulator activates, inhibits, or interacts with αVβ8. In some embodiments, the integrin activity modulator is selected from CRB-601 and PF-06940434. In some embodiments, the integrin activity modulator activates, inhibits, or interacts with α8β1. In some embodiments, the integrin activity modulator is an anti-α8β1 antibody. In some embodiments, the anti-α8β1 antibody is an anti-α8β1 antibody disclosed in WO2011049082. In some embodiments, the integrin activity modulator activates, inhibits, or interacts with αIIbβ3. In some embodiments, the integrin activity modulator is selected from eptifibatide, RUC-1, Tc-99m-P280, tirofiban, zalunfiban, and an antibody that interacts with αIIbβ3. In some embodiments, the PD-1 inhibitor or PD-L1 inhibitor is an anti-PD-1 antibody. In some embodiments, the anti-PD-1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), PF-06801591, MGD-019 (PD-1/CTLA4), KN-046 (PD-1/CTLA4), XmAb-20717 (PD-1/CTLA4), AK-104 (CTLA4/PD-1), and MEDI-5752 (CTLA4/PD-1). In some embodiments, the anti-PD-1 antibody is zimberelimab. In some embodiments, the PD-1 inhibitor or PD-L1 inhibitor is an anti-PD-L1 antibody. In some embodiments, the PD-L1 inhibitor is selected from the group consisting of atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the PD-L1 inhibitor is a small molecule inhibitor. In some embodiments, the small molecule PD-L1 inhibitor is selected from the group consisting of CA-170, GS-4224, GS-4416, INCB99280, INCB99318, and lazertinib. In some embodiments, the PD-1 inhibitor or PD-L1 inhibitor is atezolizumab. In some embodiments, the PD-1 inhibitor or PD-L1 inhibitor is durvalumab. In some embodiments, the PD-1 inhibitor or PD-L1 inhibitor is MK-7684A. In some embodiments, the PD-1 inhibitor or PD-L1 inhibitor is nivolumab. In some embodiments, the PD-1 inhibitor or PD-L1 inhibitor is pembrolizumab. In some embodiments, the PD-1 inhibitor or PD-L1 inhibitor is zimberelimab. In some embodiments, the cancer is a solid tumor. In some embodiments, the cancer is selected from breast cancer or pancreatic cancer. In some embodiments, the cancer is breast cancer. In some embodiments, the breast cancer is triple-negative breast cancer (TNBC), HR+/HER2− breast cancer (HR+/HER2− BC), HR+/HER2low breast cancer (HR+/HER2low BC). In some embodiments, the cancer is pancreatic cancer. In some embodiments, the pancreatic cancer is pancreatic ductal adenocarcinoma (PDAC). In some embodiments, the cancer is metastatic. In some embodiments, the subject is human. In some embodiments, the subject is treatment naïve. In some embodiments, the subject has received at least one previous anticancer treatment selected from the group consisting of surgery, radiation therapy, chemotherapy, or checkpoint inhibitor therapy. In some embodiments, the cancer has progressed or recurred following an anticancer treatment selected from the group consisting of surgery, radiation therapy, chemotherapy, or checkpoint inhibitor therapy. In some embodiments, the cancer is resistant or refractory to checkpoint inhibitor therapy. In some embodiments, the checkpoint inhibitor therapy comprises an anti-PD-1 antibody or anti-PD-L1 antibody. In some embodiments, the anti-PD-1 antibody or anti-PD-L1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), MGD-019 (PD-1/CTLA4), KN-046 (PD-1/CTLA4), XmAb-20717 (PD-1/CTLA4), AK-104 (CTLA4/PD-1), MEDI-5752 (CTLA4/PD-1), atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, atezolizumab, avelumab, envafolimab, durvalumab, cosibelimab, lodapolimab, garivulimab, envafolimab, opucolimab, and manelimab. In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, and vopratelimab. In some embodiments, the chemotherapy is selected from the group consisting of 5-FU, leucovorin, oxaliplatin, irinotecan, gemcitabine, nab-paclitaxel (Abraxane®), FOLFIRINOX, and combinations thereof. In some embodiments, therapeutic agents used to treat pancreatic cancer comprise a combination of (a) 5-FU+leucovorin+oxaliplatin+irinotecan, (b) 5-FU+nanoliposomal irinotecan, (c) leucovorin+nanoliposomal irinotecan, or (d) gemcitabine+nab-paclitaxel. In some embodiments, the kit further comprises an additional therapeutic agent or therapeutic modality.

Further disclosed herein are kits for use in the treatment, mitigation, reduction, prevention, or delay of the recurrence or metastasis of cancer, wherein the kit comprises (a) an integrin activity modulator; and (b) a PD-1 inhibitor or PD-L1 inhibitor, wherein (i) the integrin activity modulator is selected from bexotegrast (i.e., PLN-74809), PLN-1474, and PLN-1561; or (ii) the PD-1 inhibitor or PD-L1 inhibitor is selected from atezolizumab, durvalumab, MK-7684A, nivolumab, pembrolizumab, and zimberelimab. In some embodiments, the integrin activity modulator is selected from an integrin-activating agent, an integrin-inhibiting agent, or an agent that is capable of interacting with one or more integrins. In some embodiments, the integrin activity modulator is a small molecule or a large molecule. In some embodiments, the large molecule is an antibody or antibody fragment. In some embodiments, the large molecule is an inhibitory peptide or peptide antagonist. In some embodiments, the integrin activity modulator activates, inhibits, or interacts with an integrin alpha (α) subunit. In some embodiments, the integrin a subunit is selected from α5, αV, α8, and αIIb. In some embodiments, the integrin activity modulator activates, inhibits, or interacts with the αV subunit. In some embodiments, the integrin activity modulator is an anti-αV antibody. In some embodiments, the anti-αV antibody is selected from abituzumab and intetumumab. In some embodiments, the integrin activity modulator is an iRGD peptide. In some embodiments, the iRGD peptide is CEND-1. In some embodiments, the integrin activity modulator activates, inhibits, or interacts with an integrin beta (β) subunit. In some embodiments, the β subunit is selected from β1, β3, β5, and β8. In some embodiments, the integrin activity modulator activates, inhibits, or interacts with β1. In some embodiments, the integrin activity modulator is an anti-β1 antibody. In some embodiments, the anti-β1 antibody is OS2966. In some embodiments, the integrin activity modulator activates, inhibits, or interacts with β5. In some embodiments, the integrin activity modulator is an anti-β5 antibody. In some embodiments, the anti-β5 antibody is ALULA. In some embodiments, the integrin activity modulator activates, inhibits, or interacts with an RGD-binding integrin. In some embodiments, RGD-binding integrin is selected from α5β1, αVβ1, αVβ3, αVβ5, αVβ6, αVβ8, α8β1, and αIIbβ3. In some embodiments, the integrin activity modulator activates, inhibits, or interacts with α5β1. In some embodiments, the integrin activity modulator is selected from AG-73305, AP25, AMEP, ATN-161, F-200 (Eos), JSM-6427, liposomal contortrostatin, PF-04605412, bexotegrast (i.e., PLN-74809), and SJ-749. In some embodiments, the integrin activity modulator activates, inhibits, or interacts with αVβ1. In some embodiments, the integrin activity modulator is selected from bexotegrast (i.e., PLN-74809), IDL-2965, PLN-1474, PLN-1561, and volociximab. In some embodiments, the integrin activity modulator is selected from bexotegrast (i.e., PLN-74809), PLN-1474, and PLN-1561. In some embodiments, the integrin activity modulator is bexotegrast. In some embodiments, the integrin activity modulator is PLN-1474. In some embodiments, the integrin activity modulator is PLN-1561. In some embodiments, the integrin activity modulator activates, inhibits, or interacts with αVβ3. In some embodiments, the integrin activity modulator is selected from AP25, B-1451, BS-1417, cilengitide, CT-6725, efatucizumab, etaracizumab, FF-10158, HM-3, IPS-02001, IPS-02101, IPS-05002, JNJ-26076713, MK-0429, NTA-1, NVX-188, Ocurin, ProAgio, PS-388023, RP-697, RP-748, SB-273005, Sch-221153, SC-69000, SD-983, SF-0166, SM-256, SOM-0777, SQ-885, TSRI-265, VIP-236, vitaxin, VPI-2690B, and XT-199. In some embodiments, the integrin activity modulator activates, inhibits, or interacts with αVβ5. In some embodiments, the integrin activity modulator is selected from ALULA, SNAKA-51, and STX-200. In some embodiments, the integrin activity modulator activates, inhibits, or interacts with αVβ6. In some embodiments, the integrin activity modulator is selected from 264-RAD, A20FMDV2, abituzumab, ADVCA0848, bexotegrast (i.e., PLN-74809), CRB-602, GSK-3008348, IDL-2965, intetumumab, MORF-627, MORF-720, PLN-1177, PLN-1561, PLN-1705, SGN-B6A, and STX-100. In some embodiments, the integrin activity modulator activates, inhibits, or interacts with αVβ8. In some embodiments, the integrin activity modulator is selected from CRB-601 and PF-06940434. In some embodiments, the integrin activity modulator activates, inhibits, or interacts with α8β1. In some embodiments, the integrin activity modulator is an anti-α8β1 antibody. In some embodiments, the anti-α8β1 antibody is an anti-α8β1 antibody disclosed in WO2011049082. In some embodiments, the integrin activity modulator activates, inhibits, or interacts with αIIbβ3. In some embodiments, the integrin activity modulator is selected from eptifibatide, RUC-1, Tc-99m-P280, tirofiban, zalunfiban, and an antibody that interacts with αIIbβ3. In some embodiments, the PD-1 inhibitor or PD-L1 inhibitor is an anti-PD-1 antibody. In some embodiments, the anti-PD-1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), PF-06801591, MGD-019 (PD-1/CTLA4), KN-046 (PD-1/CTLA4), XmAb-20717 (PD-1/CTLA4), AK-104 (CTLA4/PD-1), and MEDI-5752 (CTLA4/PD-1). In some embodiments, the anti-PD-1 antibody is zimberelimab. In some embodiments, the PD-1 inhibitor or PD-L1 inhibitor is an anti-PD-L1 antibody. In some embodiments, the PD-L1 inhibitor is selected from the group consisting of atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the PD-L1 inhibitor is a small molecule inhibitor. In some embodiments, the small molecule PD-L1 inhibitor is selected from the group consisting of CA-170, GS-4224, GS-4416, INCB99280, INCB99318, and lazertinib. In some embodiments, the PD-1 inhibitor or PD-L1 inhibitor is atezolizumab. In some embodiments, the PD-1 inhibitor or PD-L1 inhibitor is durvalumab. In some embodiments, the PD-1 inhibitor or PD-L1 inhibitor is MK-7684A. In some embodiments, the PD-1 inhibitor or PD-L1 inhibitor is nivolumab. In some embodiments, the PD-1 inhibitor or PD-L1 inhibitor is pembrolizumab. In some embodiments, the PD-1 inhibitor or PD-L1 inhibitor is zimberelimab. In some embodiments, the cancer is a solid tumor. In some embodiments, the cancer is selected from breast cancer or pancreatic cancer. In some embodiments, the cancer is breast cancer. In some embodiments, the breast cancer is triple-negative breast cancer (TNBC), HR+/HER2− breast cancer (HR+/HER2− BC), HR+/HER2low breast cancer (HR+/HER2low BC). In some embodiments, the cancer is pancreatic cancer. In some embodiments, the pancreatic cancer is pancreatic ductal adenocarcinoma (PDAC). In some embodiments, the cancer is metastatic. In some embodiments, the subject is human. In some embodiments, the subject is treatment naïve. In some embodiments, the subject has received at least one previous anticancer treatment selected from the group consisting of surgery, radiation therapy, chemotherapy, or checkpoint inhibitor therapy. In some embodiments, the cancer has progressed or recurred following an anticancer treatment selected from the group consisting of surgery, radiation therapy, chemotherapy, or checkpoint inhibitor therapy. In some embodiments, the cancer is resistant or refractory to checkpoint inhibitor therapy. In some embodiments, the checkpoint inhibitor therapy comprises an anti-PD-1 antibody or anti-PD-L1 antibody. In some embodiments, the anti-PD-1 antibody or anti-PD-L1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), MGD-019 (PD-1/CTLA4), KN-046 (PD-1/CTLA4), XmAb-20717 (PD-1/CTLA4), AK-104 (CTLA4/PD-1), MEDI-5752 (CTLA4/PD-1), atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, atezolizumab, avelumab, envafolimab, durvalumab, cosibelimab, lodapolimab, garivulimab, envafolimab, opucolimab, and manelimab. In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, and vopratelimab. In some embodiments, the chemotherapy is selected from the group consisting of 5-FU, leucovorin, oxaliplatin, irinotecan, gemcitabine, nab-paclitaxel (Abraxane®), FOLFIRINOX, and combinations thereof. In some embodiments, therapeutic agents used to treat pancreatic include cancer combinations of (a) 5-FU+leucovorin+oxaliplatin+irinotecan, (b) 5-FU+nanoliposomal irinotecan, (c) leucovorin+nanoliposomal irinotecan, or (d) gemcitabine+nab-paclitaxel. In some embodiments, the kit further comprises an additional therapeutic agent or therapeutic modality.

Further disclosed herein are kits for use as a medicament, wherein the kit comprises (a) an integrin activity modulator selected from bexotegrast (i.e., PLN-74809), PLN-1474, and PLN-1561; and (b) a PD-1 inhibitor or PD-L1 inhibitor selected from atezolizumab, durvalumab, MK-7684A, nivolumab, pembrolizumab, and zimberelimab. In some embodiments, the integrin activity modulator is bexotegrast. In some embodiments, the integrin activity modulator is PLN-1474. In some embodiments, the integrin activity modulator is PLN-1561. In some embodiments, the PD-1 inhibitor or PD-L1 inhibitor is atezolizumab. In some embodiments, the PD-1 inhibitor or PD-L1 inhibitor is durvalumab. In some embodiments, the PD-1 inhibitor or PD-L1 inhibitor is MK-7684A. In some embodiments, the PD-1 inhibitor or PD-L1 inhibitor is nivolumab. In some embodiments, the PD-1 inhibitor or PD-L1 inhibitor is pembrolizumab. In some embodiments, the PD-1 inhibitor or PD-L1 inhibitor is zimberelimab. In some embodiments, the breast cancer is triple-negative breast cancer (TNBC), HR+/HER2− breast cancer (HR+/HER2− BC), HR+/HER2low breast cancer (HR+/HER2low BC). In some embodiments, the pancreatic cancer is pancreatic ductal adenocarcinoma (PDAC). In some embodiments, the cancer is metastatic. In some embodiments, the subject is human. In some embodiments, the subject is treatment naïve. In some embodiments, the subject has received at least one previous anticancer treatment selected from the group consisting of surgery, radiation therapy, chemotherapy, or checkpoint inhibitor therapy. In some embodiments, the cancer has progressed or recurred following an anticancer treatment selected from the group consisting of surgery, radiation therapy, chemotherapy, or checkpoint inhibitor therapy. In some embodiments, the cancer is resistant or refractory to checkpoint inhibitor therapy. In some embodiments, the checkpoint inhibitor therapy comprises an anti-PD-1 antibody or anti-PD-L1 antibody. In some embodiments, the anti-PD-1 antibody or anti-PD-L1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), MGD-019 (PD-1/CTLA4), KN-046 (PD-1/CTLA4), XmAb-20717 (PD-1/CTLA4), AK-104 (CTLA4/PD-1), MEDI-5752 (CTLA4/PD-1), atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, atezolizumab, avelumab, envafolimab, durvalumab, cosibelimab, lodapolimab, garivulimab, envafolimab, opucolimab, and manelimab. In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, and vopratelimab. In some embodiments, the chemotherapy is selected from the group consisting of 5-FU, leucovorin, oxaliplatin, irinotecan, gemcitabine, nab-paclitaxel (Abraxane®), FOLFIRINOX, and combinations thereof. In some embodiments, therapeutic agents used to treat pancreatic include cancer combinations of (a) 5-FU+leucovorin+oxaliplatin+irinotecan, (b) 5-FU+nanoliposomal irinotecan, (c) leucovorin+nanoliposomal irinotecan, or (d) gemcitabine+nab-paclitaxel. In some embodiments, the kit further comprises an additional therapeutic agent or therapeutic modality.

Further disclosed herein are kits for use in the treatment, mitigation, reduction, prevention, or delay of the recurrence or metastasis of pancreatic cancer or breast cancer, wherein the kit comprises (a) an integrin activity modulator selected from bexotegrast (i.e., PLN-74809), PLN-1474, and PLN-1561; and (b) a PD-1 inhibitor or PD-L1 inhibitor selected from atezolizumab, durvalumab, MK-7684A, nivolumab, pembrolizumab, and zimberelimab. In some embodiments, the integrin activity modulator is bexotegrast. In some embodiments, the integrin activity modulator is PLN-1474. In some embodiments, the integrin activity modulator is PLN-1561. In some embodiments, the PD-1 inhibitor or PD-L1 inhibitor is atezolizumab. In some embodiments, the PD-1 inhibitor or PD-L1 inhibitor is durvalumab. In some embodiments, the PD-1 inhibitor or PD-L1 inhibitor is MK-7684A. In some embodiments, the PD-1 inhibitor or PD-L1 inhibitor is nivolumab. In some embodiments, the PD-1 inhibitor or PD-L1 inhibitor is pembrolizumab. In some embodiments, the PD-1 inhibitor or PD-L1 inhibitor is zimberelimab. In some embodiments, the breast cancer is triple-negative breast cancer (TNBC), HR+/HER2− breast cancer (HR+/HER2− BC), HR+/HER2low breast cancer (HR+/HER2low BC). In some embodiments, the pancreatic cancer is pancreatic ductal adenocarcinoma (PDAC). In some embodiments, the cancer is metastatic. In some embodiments, the subject is human. In some embodiments, the subject is treatment naïve. In some embodiments, the subject has received at least one previous anticancer treatment selected from the group consisting of surgery, radiation therapy, chemotherapy, or checkpoint inhibitor therapy. In some embodiments, the cancer has progressed or recurred following an anticancer treatment selected from the group consisting of surgery, radiation therapy, chemotherapy, or checkpoint inhibitor therapy. In some embodiments, the cancer is resistant or refractory to checkpoint inhibitor therapy. In some embodiments, the checkpoint inhibitor therapy comprises an anti-PD-1 antibody or anti-PD-L1 antibody. In some embodiments, the anti-PD-1 antibody or anti-PD-L1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), MGD-019 (PD-1/CTLA4), KN-046 (PD-1/CTLA4), XmAb-20717 (PD-1/CTLA4), AK-104 (CTLA4/PD-1), MEDI-5752 (CTLA4/PD-1), atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, atezolizumab, avelumab, envafolimab, durvalumab, cosibelimab, lodapolimab, garivulimab, envafolimab, opucolimab, and manelimab. In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, and vopratelimab. In some embodiments, the chemotherapy is selected from the group consisting of 5-FU, leucovorin, oxaliplatin, irinotecan, gemcitabine, nab-paclitaxel (Abraxane®), FOLFIRINOX, and combinations thereof. In some embodiments, therapeutic agents used to treat pancreatic include cancer combinations of (a) 5-FU+leucovorin+oxaliplatin+irinotecan, (b) 5-FU+nanoliposomal irinotecan, (c) leucovorin+nanoliposomal irinotecan, or (d) gemcitabine+nab-paclitaxel. In some embodiments, the kit further comprises an additional therapeutic agent or therapeutic modality.

### Doses, Dosing Regimens, and Routes of Administration

In some embodiments, any of the methods or kits disclosed herein comprise one or more integrin activity modulators. In some embodiments, the dose of the one or more integrin activity modulators is at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg.

In some embodiments, the dose of the one or more integrin activity modulators is between about 5 mg to about 500 mg, about 10 mg to about 500 mg, about 15 mg to about 500 mg, about 20 mg to about 500 mg, about 25 mg to about 500 mg, about 30 mg to about 500 mg, about 35 mg to about 500 mg, about 40 mg to about 500 mg, about 45 mg to about 500 mg, about 50 mg to about 500 mg, about 55 mg to about 500 mg, about 60 mg to about 500 mg, about 65 mg to about 500 mg, about 70 mg to about 500 mg, about 75 mg to about 500 mg, about 80 mg to about 500 mg, about 85 mg to about 500 mg, about 90 mg to about 500 mg, about 95 mg to about 500 mg, about 100 mg to about 500 mg, about 110 mg to about 500 mg, about 120 mg to about 500 mg, about 130 mg to about 500 mg, about 140 mg to about 500 mg, about 150 mg to about 500 mg, about 160 mg to about 500 mg, about 170 mg to about 500 mg, about 180 mg to about 500 mg, about 190 mg to about 500 mg, about 200 mg to about 500 mg, about 210 mg to about 500 mg, about 220 mg to about 500 mg, about 230 mg to about 500 mg, about 240 mg to about 500 mg, about 250 mg to about 500 mg, about 260 mg to about 500 mg, about 270 mg to about 500 mg, about 280 mg to about 500 mg, about 290 mg to about 500 mg, or about 300 mg to about 500 mg.

In some embodiments, the dose of the one or more integrin activity modulators is between about 5 mg to about 400 mg, about 10 mg to about 400 mg, about 15 mg to about 400 mg, about 20 mg to about 400 mg, about 25 mg to about 400 mg, about 30 mg to about 400 mg, about 35 mg to about 400 mg, about 40 mg to about 400 mg, about 45 mg to about 400 mg, about 50 mg to about 400 mg, about 55 mg to about 400 mg, about 60 mg to about 400 mg, about 65 mg to about 400 mg, about 70 mg to about 400 mg, about 75 mg to about 400 mg, about 80 mg to about 400 mg, about 85 mg to about 400 mg, about 90 mg to about 400 mg, about 95 mg to about 400 mg, about 100 mg to about 400 mg, about 110 mg to about 400 mg, about 120 mg to about 400 mg, about 130 mg to about 400 mg, about 140 mg to about 400 mg, about 150 mg to about 400 mg, about 160 mg to about 400 mg, about 170 mg to about 400 mg, about 180 mg to about 400 mg, about 190 mg to about 400 mg, about 200 mg to about 400 mg, about 210 mg to about 400 mg, about 220 mg to about 400 mg, about 230 mg to about 400 mg, about 240 mg to about 400 mg, about 250 mg to about 400 mg, about 260 mg to about 400 mg, about 270 mg to about 400 mg, about 280 mg to about 400 mg, about 290 mg to about 400 mg, or about 300 mg to about 400 mg.

In some embodiments, the dose of the one or more integrin activity modulators is between about 5 mg to about 300 mg, about 10 mg to about 300 mg, about 15 mg to about 300 mg, about 20 mg to about 300 mg, about 25 mg to about 300 mg, about 30 mg to about 300 mg, about 35 mg to about 300 mg, about 40 mg to about 300 mg, about 45 mg to about 300 mg, about 50 mg to about 300 mg, about 55 mg to about 300 mg, about 60 mg to about 300 mg, about 65 mg to about 300 mg, about 70 mg to about 300 mg, about 75 mg to about 300 mg, about 80 mg to about 300 mg, about 85 mg to about 300 mg, about 90 mg to about 300 mg, about 95 mg to about 300 mg, about 100 mg to about 300 mg, about 110 mg to about 300 mg, about 120 mg to about 300 mg, about 130 mg to about 300 mg, about 140 mg to about 300 mg, about 150 mg to about 300 mg, about 160 mg to about 300 mg, about 170 mg to about 300 mg, about 180 mg to about 300 mg, about 190 mg to about 300 mg, about 200 mg to about 300 mg, about 210 mg to about 300 mg, about 220 mg to about 300 mg, about 230 mg to about 300 mg, about 240 mg to about 300 mg, about 250 mg to about 300 mg, about 260 mg to about 300 mg, about 270 mg to about 300 mg, about 280 mg to about 300 mg, or about 290 mg to about 300 mg.

In some embodiments, the dose of the integrin activity modulator is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg.

In some embodiments, the dose of the one or more integrin activity modulators is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg.

In some embodiments, the one or more integrin activity modulators is administered in at least 2, 3, 4, 5, 6, 7, 8, 9, 10, 11, 12, 13, 14, 15, 16, 17, 18, 19, or 20 or more doses. In some embodiments, the 2, 3, 4, 5, 6, 7, 8, 9, 10, 11, 12, 13, 14, 15, 16, 17, 18, 19, or 20 or more doses are the same. In some embodiments, the 2, 3, 4, 5, 6, 7, 8, 9, 10, 11, 12, 13, 14, 15, 16, 17, 18, 19, or 20 or more doses are different.

In some embodiments, the integrin activity modulator is administered daily. In some embodiments, two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 days apart. In some embodiments, two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks apart. In some embodiments, two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 months apart.

In some embodiments, the dose of bexotegrast is at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg.

In some embodiments, the dose of bexotegrast is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg.

In some embodiments, the dose of bexotegrast is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg.

In some embodiments, bexotegrast is administered daily.

In some embodiments, bexotegrast is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. In some embodiments, bexotegrast is administered orally.

In some embodiments, any of the methods or kits disclosed herein comprise one or more PD-1 or PD-L1 inhibitors. In some embodiments, the dose of the PD-1 inhibitor or PD-L1 inhibitor is at least about 30 mg, 60 mg, 120 mg, 240 mg, 360 mg, 480 mg, 600 mg, 720 mg, 840 mg, 960 mg, 1080 mg, or 1200 mg.

In some embodiments, the dose of the PD-1 inhibitor or PD-L1 inhibitor is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg.

In some embodiments, the dose of the PD-1 inhibitor or PD-L1 inhibitor is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg.

In some embodiments, the PD-1 inhibitor or PD-L1 inhibitor is administered daily. In some embodiments, two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 days apart. In some embodiments, two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks apart. In some embodiments, two or more doses of the PD-1 inhibitor or PD-L1 inhibitor are administered at least 3 weeks about. In some embodiments, two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 months apart.

In some embodiments, the PD-1 inhibitor or PD-L1 inhibitor is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions.

In some embodiments, the dose of zimberelimab is at least about 30 mg, 60 mg, 120 mg, 240 mg, 360 mg, 480 mg, 600 mg, 720 mg, 840 mg, 960 mg, 1080 mg, or 1200 mg. In some embodiments, the dose of zimberelimab is at least about 360 mg.

In some embodiments, the dose of zimberelimab is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg.

In some embodiments, the dose of zimberelimab is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg.

In some embodiments, two or more doses of zimberelimab are administered at least about 1, 2, 3, 4, 5, 6, 7, 8, 9 10, 11, or 12 weeks apart. In some embodiments, two or more doses of zimberelimab are administered at least about every 3 weeks.

In some embodiments, zimberelimab is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. In some embodiments, zimberelimab is administered intravenously. In some embodiments, zimberelimab is administered subcutaneously.

### EXEMPLARY EMBODIMENTS

In some embodiments, provided herein are methods of treating, mitigating, reducing, preventing or delaying the recurrence or metastasis of cancer in a subject comprising co-administering to the subject an effective amount of (a) an integrin activity modulator; and (b) a PD-1 inhibitor or PD-L1 inhibitor. In some embodiments, the integrin activity modulator is selected from an integrin-activating agent, an integrin-inhibiting agent, or an agent that is capable of interacting with one or more integrins. In some embodiments, the integrin activity modulator is a small molecule or a large molecule. In some embodiments, the large molecule is an antibody or antibody fragment. In some embodiments, the large molecule is an inhibitory peptide or peptide antagonist. In some embodiments, the integrin activity modulator activates, inhibits, or interacts with an integrin alpha (α) subunit. In some embodiments, the integrin a subunit is selected from α5, αV, α8, and αIIb. In some embodiments, the integrin activity modulator activates, inhibits, or interacts with the αV subunit. In some embodiments, the integrin activity modulator is an anti-αV antibody. In some embodiments, the anti-αV antibody is selected from abituzumab and intetumumab. In some embodiments, the integrin activity modulator is an iRGD peptide. In some embodiments, the iRGD peptide is CEND-1. In some embodiments, the integrin activity modulator activates, inhibits, or interacts with an integrin beta (β) subunit. In some embodiments, the β subunit is selected from β1, β3, β5, and β8. In some embodiments, the integrin activity modulator activates, inhibits, or interacts with β1. In some embodiments, the integrin activity modulator is an anti-β1 antibody. In some embodiments, the anti-β1 antibody is OS2966. In some embodiments, the integrin activity modulator activates, inhibits, or interacts with β5. In some embodiments, the integrin activity modulator is an anti-β5 antibody. In some embodiments, the anti-β5 antibody is ALULA. In some embodiments, the integrin activity modulator activates, inhibits, or interacts with an RGD-binding integrin. In some embodiments, RGD-binding integrin is selected from α5β1, αVβ1, αVβ3, αVβ5, αVβ6, αVβ8, α8β1, and αIIbβ3. In some embodiments, the integrin activity modulator activates, inhibits, or interacts with α5β1. In some embodiments, the integrin activity modulator is selected from AG-73305, AP25, AMEP, ATN-161, F-200 (Eos), JSM-6427, liposomal contortrostatin, PF-04605412, bexotegrast (i.e., PLN-74809), and SJ-749. In some embodiments, the integrin activity modulator activates, inhibits, or interacts with αVβ1. In some embodiments, the integrin activity modulator is selected from bexotegrast (i.e., PLN-74809), IDL-2965, PLN-1474, PLN-1561, and volociximab. In some embodiments, the integrin activity modulator is selected from bexotegrast (i.e., PLN-74809), PLN-1474, and PLN-1561. In some embodiments, the integrin activity modulator is bexotegrast. In some embodiments, the integrin activity modulator is PLN-1474. In some embodiments, the integrin activity modulator is PLN-1561. In some embodiments, the integrin activity modulator activates, inhibits, or interacts with αVβ3. In some embodiments, the integrin activity modulator is selected from AP25, B-1451, BS-1417, cilengitide, CT-6725, efatucizumab, etaracizumab, FF-10158, HM-3, IPS-02001, IPS-02101, IPS-05002, JNJ-26076713, MK-0429, NTA-1, NVX-188, Ocurin, ProAgio, PS-388023, RP-697, RP-748, SB-273005, Sch-221153, SC-69000, SD-983, SF-0166, SM-256, SOM-0777, SQ-885, TSRI-265, VIP-236, vitaxin, VPI-2690B, and XT-199. In some embodiments, the integrin activity modulator activates, inhibits, or interacts with αVβ5. In some embodiments, the integrin activity modulator is selected from ALULA, SNAKA-51, and STX-200. In some embodiments, the integrin activity modulator activates, inhibits, or interacts with αVβ6. In some embodiments, the integrin activity modulator is selected from 264-RAD, A20FMDV2, abituzumab, ADVCA0848, bexotegrast (i.e., PLN-74809), CRB-602, GSK-3008348, IDL-2965, intetumumab, MORF-627, MORF-720, PLN-1177, PLN-1561, PLN-1705, SGN-B6A, and STX-100. In some embodiments, the integrin activity modulator activates, inhibits, or interacts with αVβ8. In some embodiments, the integrin activity modulator is selected from CRB-601 and PF-06940434. In some embodiments, the integrin activity modulator activates, inhibits, or interacts with α8β1. In some embodiments, the integrin activity modulator is an anti-α8β1 antibody. In some embodiments, the anti-α8β1 antibody is an anti-α8β1 antibody disclosed in WO2011049082. In some embodiments, the integrin activity modulator activates, inhibits, or interacts with αIIbβ3. In some embodiments, the integrin activity modulator is selected from eptifibatide, RUC-1, Tc-99m-P280, tirofiban, zalunfiban, and an antibody that interacts with αIIbβ3. In some embodiments, the PD-1 inhibitor or PD-L1 inhibitor is an anti-PD-1 antibody. In some embodiments, the anti-PD-1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), PF-06801591, MGD-019 (PD-1/CTLA4), KN-046 (PD-1/CTLA4), XmAb-20717 (PD-1/CTLA4), AK-104 (CTLA4/PD-1), and MEDI-5752 (CTLA4/PD-1). In some embodiments, the anti-PD-1 antibody is zimberelimab. In some embodiments, the PD-1 inhibitor or PD-L1 inhibitor is an anti-PD-L1 antibody. In some embodiments, the PD-L1 inhibitor is selected from the group consisting of atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the PD-L1 inhibitor is a small molecule inhibitor. In some embodiments, the small molecule PD-L1 inhibitor is selected from the group consisting of CA-170, GS-4224, GS-4416, INCB99280, INCB99318, and lazertinib. In some embodiments, the PD-1 inhibitor or PD-L1 inhibitor is atezolizumab. In some embodiments, the PD-1 inhibitor or PD-L1 inhibitor is durvalumab. In some embodiments, the PD-1 inhibitor or PD-L1 inhibitor is MK-7684A. In some embodiments, the PD-1 inhibitor or PD-L1 inhibitor is nivolumab. In some embodiments, the PD-1 inhibitor or PD-L1 inhibitor is pembrolizumab. In some embodiments, the PD-1 inhibitor or PD-L1 inhibitor is zimberelimab. In some embodiments, the dose of the one or more integrin activity modulators is at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg. In some embodiments, the dose of the one or more integrin activity modulators is between about 5 mg to about 500 mg, about 10 mg to about 500 mg, about 15 mg to about 500 mg, about 20 mg to about 500 mg, about 25 mg to about 500 mg, about 30 mg to about 500 mg, about 35 mg to about 500 mg, about 40 mg to about 500 mg, about 45 mg to about 500 mg, about 50 mg to about 500 mg, about 55 mg to about 500 mg, about 60 mg to about 500 mg, about 65 mg to about 500 mg, about 70 mg to about 500 mg, about 75 mg to about 500 mg, about 80 mg to about 500 mg, about 85 mg to about 500 mg, about 90 mg to about 500 mg, about 95 mg to about 500 mg, about 100 mg to about 500 mg, about 110 mg to about 500 mg, about 120 mg to about 500 mg, about 130 mg to about 500 mg, about 140 mg to about 500 mg, about 150 mg to about 500 mg, about 160 mg to about 500 mg, about 170 mg to about 500 mg, about 180 mg to about 500 mg, about 190 mg to about 500 mg, about 200 mg to about 500 mg, about 210 mg to about 500 mg, about 220 mg to about 500 mg, about 230 mg to about 500 mg, about 240 mg to about 500 mg, about 250 mg to about 500 mg, about 260 mg to about 500 mg, about 270 mg to about 500 mg, about 280 mg to about 500 mg, about 290 mg to about 500 mg, or about 300 mg to about 500 mg. In some embodiments, the dose of the one or more integrin activity modulators is between about 5 mg to about 400 mg, about 10 mg to about 400 mg, about 15 mg to about 400 mg, about 20 mg to about 400 mg, about 25 mg to about 400 mg, about 30 mg to about 400 mg, about 35 mg to about 400 mg, about 40 mg to about 400 mg, about 45 mg to about 400 mg, about 50 mg to about 400 mg, about 55 mg to about 400 mg, about 60 mg to about 400 mg, about 65 mg to about 400 mg, about 70 mg to about 400 mg, about 75 mg to about 400 mg, about 80 mg to about 400 mg, about 85 mg to about 400 mg, about 90 mg to about 400 mg, about 95 mg to about 400 mg, about 100 mg to about 400 mg, about 110 mg to about 400 mg, about 120 mg to about 400 mg, about 130 mg to about 400 mg, about 140 mg to about 400 mg, about 150 mg to about 400 mg, about 160 mg to about 400 mg, about 170 mg to about 400 mg, about 180 mg to about 400 mg, about 190 mg to about 400 mg, about 200 mg to about 400 mg, about 210 mg to about 400 mg, about 220 mg to about 400 mg, about 230 mg to about 400 mg, about 240 mg to about 400 mg, about 250 mg to about 400 mg, about 260 mg to about 400 mg, about 270 mg to about 400 mg, about 280 mg to about 400 mg, about 290 mg to about 400 mg, or about 300 mg to about 400 mg. In some embodiments, the dose of the one or more integrin activity modulators is between about 5 mg to about 300 mg, about 10 mg to about 300 mg, about 15 mg to about 300 mg, about 20 mg to about 300 mg, about 25 mg to about 300 mg, about 30 mg to about 300 mg, about 35 mg to about 300 mg, about 40 mg to about 300 mg, about 45 mg to about 300 mg, about 50 mg to about 300 mg, about 55 mg to about 300 mg, about 60 mg to about 300 mg, about 65 mg to about 300 mg, about 70 mg to about 300 mg, about 75 mg to about 300 mg, about 80 mg to about 300 mg, about 85 mg to about 300 mg, about 90 mg to about 300 mg, about 95 mg to about 300 mg, about 100 mg to about 300 mg, about 110 mg to about 300 mg, about 120 mg to about 300 mg, about 130 mg to about 300 mg, about 140 mg to about 300 mg, about 150 mg to about 300 mg, about 160 mg to about 300 mg, about 170 mg to about 300 mg, about 180 mg to about 300 mg, about 190 mg to about 300 mg, about 200 mg to about 300 mg, about 210 mg to about 300 mg, about 220 mg to about 300 mg, about 230 mg to about 300 mg, about 240 mg to about 300 mg, about 250 mg to about 300 mg, about 260 mg to about 300 mg, about 270 mg to about 300 mg, about 280 mg to about 300 mg, or about 290 mg to about 300 mg. In some embodiments, the dose of the integrin activity modulator is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, the dose of the one or more integrin activity modulators is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the one or more integrin activity modulators is administered in at least 2, 3, 4, 5, 6, 7, 8, 9, 10, 11, 12, 13, 14, 15, 16, 17, 18, 19, or 20 or more doses. In some embodiments, the 2, 3, 4, 5, 6, 7, 8, 9, 10, 11, 12, 13, 14, 15, 16, 17, 18, 19, or 20 or more doses are the same. In some embodiments, the 2, 3, 4, 5, 6, 7, 8, 9, 10, 11, 12, 13, 14, 15, 16, 17, 18, 19, or 20 or more doses are different. In some embodiments, the integrin activity modulator is administered daily. In some embodiments, two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 days apart. In some embodiments, two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks apart. In some embodiments, two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 months apart. In some embodiments, the dose of bexotegrast is at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg. In some embodiments, the dose of bexotegrast is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the dose of bexotegrast is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, bexotegrast is administered daily. In some embodiments, bexotegrast is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. In some embodiments, bexotegrast is administered orally. In some embodiments, any of the methods or kits disclosed herein comprise one or more PD-1 or PD-L1 inhibitors. In some embodiments, the dose of the PD-1 inhibitor or PD-L1 inhibitor is at least about 30 mg, 60 mg, 120 mg, 240 mg, 360 mg, 480 mg, 600 mg, 720 mg, 840 mg, 960 mg, 1080 mg, or 1200 mg. In some embodiments, the dose of the PD-1 inhibitor or PD-L1 inhibitor is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the dose of the PD-1 inhibitor or PD-L1 inhibitor is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, the PD-1 inhibitor or PD-L1 inhibitor is administered daily. In some embodiments, two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 days apart. In some embodiments, two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks apart. In some embodiments, two or more doses of the PD-1 inhibitor or PD-L1 inhibitor are administered at least 3 weeks about. In some embodiments, two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 months apart. In some embodiments, the PD-1 inhibitor or PD-L1 inhibitor is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularily, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. In some embodiments, the dose of zimberelimab is at least about 30 mg, 60 mg, 120 mg, 240 mg, 360 mg, 480 mg, 600 mg, 720 mg, 840 mg, 960 mg, 1080 mg, or 1200 mg. In some embodiments, the dose of zimberelimab is at least about 360 mg. In some embodiments, the dose of zimberelimab is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the dose of zimberelimab is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, two or more doses of zimberelimab are administered at least about 1, 2, 3, 4, 5, 6, 7, 8, 9 10, 11, or 12 weeks apart. In some embodiments, two or more doses of zimberelimab are administered at least about every 3 weeks. In some embodiments, zimberelimab is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularily, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. In some embodiments, zimberelimab is administered intravenously. In some embodiments, zimberelimab is administered subcutaneously. In some embodiments, the cancer is a solid tumor. In some embodiments, the cancer is selected from breast cancer or pancreatic cancer. In some embodiments, the cancer is breast cancer. In some embodiments, the breast cancer is triple-negative breast cancer (TNBC), HR+/HER2− breast cancer (HR+/HER2− BC), HR+/HER2low breast cancer (HR+/HER2low BC). In some embodiments, the cancer is pancreatic cancer. In some embodiments, the pancreatic cancer is pancreatic ductal adenocarcinoma (PDAC). In some embodiments, the cancer is metastatic. In some embodiments, the subject is human. In some embodiments, the subject is treatment naïve. In some embodiments, the subject has received at least one previous anticancer treatment selected from the group consisting of surgery, radiation therapy, chemotherapy, or checkpoint inhibitor therapy. In some embodiments, the cancer has progressed or recurred following an anticancer treatment selected from the group consisting of surgery, radiation therapy, chemotherapy, or checkpoint inhibitor therapy. In some embodiments, the cancer is resistant or refractory to checkpoint inhibitor therapy. In some embodiments, the checkpoint inhibitor therapy comprises an anti-PD-1 antibody or anti-PD-L1 antibody. In some embodiments, the anti-PD-1 antibody or anti-PD-L1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), MGD-019 (PD-1/CTLA4), KN-046 (PD-1/CTLA4), XmAb-20717 (PD-1/CTLA4), AK-104 (CTLA4/PD-1), MEDI-5752 (CTLA4/PD-1), atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, atezolizumab, avelumab, envafolimab, durvalumab, cosibelimab, lodapolimab, garivulimab, envafolimab, opucolimab, and manelimab. In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, and vopratelimab. In some embodiments, the chemotherapy is selected from the group consisting of 5-FU, leucovorin, oxaliplatin, irinotecan, gemcitabine, nab-paclitaxel (Abraxane®), FOLFIRINOX, and combinations thereof. In some embodiments, therapeutic agents used to treat pancreatic cancer comprise combinations of (a) 5-FU+leucovorin+oxaliplatin+irinotecan, (b) 5-FU+nanoliposomal irinotecan, (c) leucovorin+nanoliposomal irinotecan, or (d) gemcitabine+nab-paclitaxel. In some embodiments, the method further comprises co-administering an additional therapeutic agent or therapeutic modality.

In some embodiments, provided herein are methods for eliciting an immune response to cancer in a subject comprising co-administering to the subject an effective amount of (a) an integrin activity modulator; and (b) a PD-1 inhibitor or PD-L1 inhibitor. In some embodiments, the integrin activity modulator is selected from an integrin-activating agent, an integrin-inhibiting agent, or an agent that is capable of interacting with one or more integrins. In some embodiments, the integrin activity modulator is a small molecule or a large molecule. In some embodiments, the large molecule is an antibody or antibody fragment. In some embodiments, the large molecule is an inhibitory peptide or peptide antagonist. In some embodiments, the integrin activity modulator activates, inhibits, or interacts with an integrin alpha (α) subunit. In some embodiments, the integrin a subunit is selected from α5, αV, α8, and αIIb. In some embodiments, the integrin activity modulator activates, inhibits, or interacts with the αV subunit. In some embodiments, the integrin activity modulator is an anti-αV antibody. In some embodiments, the anti-αV antibody is selected from abituzumab and intetumumab. In some embodiments, the integrin activity modulator is an iRGD peptide. In some embodiments, the iRGD peptide is CEND-1. In some embodiments, the integrin activity modulator activates, inhibits, or interacts with an integrin beta (β) subunit. In some embodiments, the β subunit is selected from β1, β3, β5, and β8. In some embodiments, the integrin activity modulator activates, inhibits, or interacts with β1. In some embodiments, the integrin activity modulator is an anti-β1 antibody. In some embodiments, the anti-β1 antibody is OS2966. In some embodiments, the integrin activity modulator activates, inhibits, or interacts with β5. In some embodiments, the integrin activity modulator is an anti-β5 antibody. In some embodiments, the anti-β5 antibody is ALULA. In some embodiments, the integrin activity modulator activates, inhibits, or interacts with an RGD-binding integrin. In some embodiments, RGD-binding integrin is selected from α5β1, αVβ1, αVβ3, αVβ5, αVβ6, αVβ8, α8β1, and αIIbβ3. In some embodiments, the integrin activity modulator activates, inhibits, or interacts with α5β1. In some embodiments, the integrin activity modulator is selected from AG-73305, AP25, AMEP, ATN-161, F-200 (Eos), JSM-6427, liposomal contortrostatin, PF-04605412, bexotegrast (i.e., PLN-74809), and SJ-749. In some embodiments, the integrin activity modulator activates, inhibits, or interacts with αVβ1. In some embodiments, the integrin activity modulator is selected from bexotegrast (i.e., PLN-74809), IDL-2965, PLN-1474, PLN-1561, and volociximab. In some embodiments, the integrin activity modulator is selected from bexotegrast (i.e., PLN-74809), PLN-1474, and PLN-1561. In some embodiments, the integrin activity modulator is bexotegrast. In some embodiments, the integrin activity modulator is PLN-1474. In some embodiments, the integrin activity modulator is PLN-1561. In some embodiments, the integrin activity modulator activates, inhibits, or interacts with αVβ3. In some embodiments, the integrin activity modulator is selected from AP25, B-1451, BS-1417, cilengitide, CT-6725, efatucizumab, etaracizumab, FF-10158, HM-3, IPS-02001, IPS-02101, IPS-05002, JNJ-26076713, MK-0429, NTA-1, NVX-188, Ocurin, ProAgio, PS-388023, RP-697, RP-748, SB-273005, Sch-221153, SC-69000, SD-983, SF-0166, SM-256, SOM-0777, SQ-885, TSRI-265, VIP-236, vitaxin, VPI-2690B, and XT-199. In some embodiments, the integrin activity modulator activates, inhibits, or interacts with αVβ5. In some embodiments, the integrin activity modulator is selected from ALULA, SNAKA-51, and STX-200. In some embodiments, the integrin activity modulator activates, inhibits, or interacts with αVβ6. In some embodiments, the integrin activity modulator is selected from 264-RAD, A20FMDV2, abituzumab, ADVCA0848, bexotegrast (i.e., PLN-74809), CRB-602, GSK-3008348, IDL-2965, intetumumab, MORF-627, MORF-720, PLN-1177, PLN-1561, PLN-1705, SGN-B6A, and STX-100. In some embodiments, the integrin activity modulator activates, inhibits, or interacts with αVβ8. In some embodiments, the integrin activity modulator is selected from CRB-601 and PF-06940434. In some embodiments, the integrin activity modulator activates, inhibits, or interacts with α8β1. In some embodiments, the integrin activity modulator is an anti-α8β1 antibody. In some embodiments, the anti-α8β1 antibody is an anti-α8β1 antibody disclosed in WO2011049082. In some embodiments, the integrin activity modulator activates, inhibits, or interacts with αIIbβ3. In some embodiments, the integrin activity modulator is selected from eptifibatide, RUC-1, Tc-99m-P280, tirofiban, zalunfiban, and an antibody that interacts with αIIbβ3. In some embodiments, the PD-1 inhibitor or PD-L1 inhibitor is an anti-PD-1 antibody. In some embodiments, the anti-PD-1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), PF-06801591, MGD-019 (PD-1/CTLA4), KN-046 (PD-1/CTLA4), XmAb-20717 (PD-1/CTLA4), AK-104 (CTLA4/PD-1), and MEDI-5752 (CTLA4/PD-1). In some embodiments, the anti-PD-1 antibody is zimberelimab. In some embodiments, the PD-1 inhibitor or PD-L1 inhibitor is an anti-PD-L1 antibody. In some embodiments, the PD-L1 inhibitor is selected from the group consisting of atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the PD-L1 inhibitor is a small molecule inhibitor. In some embodiments, the small molecule PD-L1 inhibitor is selected from the group consisting of CA-170, GS-4224, GS-4416, INCB99280, INCB99318, and lazertinib. In some embodiments, the PD-1 inhibitor or PD-L1 inhibitor is atezolizumab. In some embodiments, the PD-1 inhibitor or PD-L1 inhibitor is durvalumab. In some embodiments, the PD-1 inhibitor or PD-L1 inhibitor is MK-7684A. In some embodiments, the PD-1 inhibitor or PD-L1 inhibitor is nivolumab. In some embodiments, the PD-1 inhibitor or PD-L1 inhibitor is pembrolizumab. In some embodiments, the PD-1 inhibitor or PD-L1 inhibitor is zimberelimab. In some embodiments, the dose of the one or more integrin activity modulators is at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg. In some embodiments, the dose of the one or more integrin activity modulators is between about 5 mg to about 500 mg, about 10 mg to about 500 mg, about 15 mg to about 500 mg, about 20 mg to about 500 mg, about 25 mg to about 500 mg, about 30 mg to about 500 mg, about 35 mg to about 500 mg, about 40 mg to about 500 mg, about 45 mg to about 500 mg, about 50 mg to about 500 mg, about 55 mg to about 500 mg, about 60 mg to about 500 mg, about 65 mg to about 500 mg, about 70 mg to about 500 mg, about 75 mg to about 500 mg, about 80 mg to about 500 mg, about 85 mg to about 500 mg, about 90 mg to about 500 mg, about 95 mg to about 500 mg, about 100 mg to about 500 mg, about 110 mg to about 500 mg, about 120 mg to about 500 mg, about 130 mg to about 500 mg, about 140 mg to about 500 mg, about 150 mg to about 500 mg, about 160 mg to about 500 mg, about 170 mg to about 500 mg, about 180 mg to about 500 mg, about 190 mg to about 500 mg, about 200 mg to about 500 mg, about 210 mg to about 500 mg, about 220 mg to about 500 mg, about 230 mg to about 500 mg, about 240 mg to about 500 mg, about 250 mg to about 500 mg, about 260 mg to about 500 mg, about 270 mg to about 500 mg, about 280 mg to about 500 mg, about 290 mg to about 500 mg, or about 300 mg to about 500 mg. In some embodiments, the dose of the one or more integrin activity modulators is between about 5 mg to about 400 mg, about 10 mg to about 400 mg, about 15 mg to about 400 mg, about 20 mg to about 400 mg, about 25 mg to about 400 mg, about 30 mg to about 400 mg, about 35 mg to about 400 mg, about 40 mg to about 400 mg, about 45 mg to about 400 mg, about 50 mg to about 400 mg, about 55 mg to about 400 mg, about 60 mg to about 400 mg, about 65 mg to about 400 mg, about 70 mg to about 400 mg, about 75 mg to about 400 mg, about 80 mg to about 400 mg, about 85 mg to about 400 mg, about 90 mg to about 400 mg, about 95 mg to about 400 mg, about 100 mg to about 400 mg, about 110 mg to about 400 mg, about 120 mg to about 400 mg, about 130 mg to about 400 mg, about 140 mg to about 400 mg, about 150 mg to about 400 mg, about 160 mg to about 400 mg, about 170 mg to about 400 mg, about 180 mg to about 400 mg, about 190 mg to about 400 mg, about 200 mg to about 400 mg, about 210 mg to about 400 mg, about 220 mg to about 400 mg, about 230 mg to about 400 mg, about 240 mg to about 400 mg, about 250 mg to about 400 mg, about 260 mg to about 400 mg, about 270 mg to about 400 mg, about 280 mg to about 400 mg, about 290 mg to about 400 mg, or about 300 mg to about 400 mg. In some embodiments, the dose of the one or more integrin activity modulators is between about 5 mg to about 300 mg, about 10 mg to about 300 mg, about 15 mg to about 300 mg, about 20 mg to about 300 mg, about 25 mg to about 300 mg, about 30 mg to about 300 mg, about 35 mg to about 300 mg, about 40 mg to about 300 mg, about 45 mg to about 300 mg, about 50 mg to about 300 mg, about 55 mg to about 300 mg, about 60 mg to about 300 mg, about 65 mg to about 300 mg, about 70 mg to about 300 mg, about 75 mg to about 300 mg, about 80 mg to about 300 mg, about 85 mg to about 300 mg, about 90 mg to about 300 mg, about 95 mg to about 300 mg, about 100 mg to about 300 mg, about 110 mg to about 300 mg, about 120 mg to about 300 mg, about 130 mg to about 300 mg, about 140 mg to about 300 mg, about 150 mg to about 300 mg, about 160 mg to about 300 mg, about 170 mg to about 300 mg, about 180 mg to about 300 mg, about 190 mg to about 300 mg, about 200 mg to about 300 mg, about 210 mg to about 300 mg, about 220 mg to about 300 mg, about 230 mg to about 300 mg, about 240 mg to about 300 mg, about 250 mg to about 300 mg, about 260 mg to about 300 mg, about 270 mg to about 300 mg, about 280 mg to about 300 mg, or about 290 mg to about 300 mg. In some embodiments, the dose of the integrin activity modulator is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, the dose of the one or more integrin activity modulators is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the one or more integrin activity modulators is administered in at least 2, 3, 4, 5, 6, 7, 8, 9, 10, 11, 12, 13, 14, 15, 16, 17, 18, 19, or 20 or more doses. In some embodiments, the 2, 3, 4, 5, 6, 7, 8, 9, 10, 11, 12, 13, 14, 15, 16, 17, 18, 19, or 20 or more doses are the same. In some embodiments, the 2, 3, 4, 5, 6, 7, 8, 9, 10, 11, 12, 13, 14, 15, 16, 17, 18, 19, or 20 or more doses are different. In some embodiments, the integrin activity modulator is administered daily. In some embodiments, two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 days apart. In some embodiments, two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks apart. In some embodiments, two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 months apart. In some embodiments, the dose of bexotegrast is at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg. In some embodiments, the dose of bexotegrast is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the dose of bexotegrast is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, bexotegrast is administered daily. In some embodiments, bexotegrast is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. In some embodiments, bexotegrast is administered orally. In some embodiments, any of the methods or kits disclosed herein comprise one or more PD-1 or PD-L1 inhibitors. In some embodiments, the dose of the PD-1 inhibitor or PD-L1 inhibitor is at least about 30 mg, 60 mg, 120 mg, 240 mg, 360 mg, 480 mg, 600 mg, 720 mg, 840 mg, 960 mg, 1080 mg, or 1200 mg. In some embodiments, the dose of the PD-1 inhibitor or PD-L1 inhibitor is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the dose of the PD-1 inhibitor or PD-L1 inhibitor is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, the PD-1 inhibitor or PD-L1 inhibitor is administered daily. In some embodiments, two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 days apart. In some embodiments, two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks apart. In some embodiments, two or more doses of the PD-1 inhibitor or PD-L1 inhibitor are administered at least 3 weeks about. In some embodiments, two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 months apart. In some embodiments, the PD-1 inhibitor or PD-L1 inhibitor is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. In some embodiments, the dose of zimberelimab is at least about 30 mg, 60 mg, 120 mg, 240 mg, 360 mg, 480 mg, 600 mg, 720 mg, 840 mg, 960 mg, 1080 mg, or 1200 mg. In some embodiments, the dose of zimberelimab is at least about 360 mg. In some embodiments, the dose of zimberelimab is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the dose of zimberelimab is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, two or more doses of zimberelimab are administered at least about 1, 2, 3, 4, 5, 6, 7, 8, 9 10, 11, or 12 weeks apart. In some embodiments, two or more doses of zimberelimab are administered at least about every 3 weeks. In some embodiments, zimberelimab is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularily, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. In some embodiments, zimberelimab is administered intravenously. In some embodiments, zimberelimab is administered subcutaneously. In some embodiments, the cancer is a solid tumor. In some embodiments, the cancer is selected from breast cancer or pancreatic cancer. In some embodiments, the cancer is breast cancer. In some embodiments, the breast cancer is triple-negative breast cancer (TNBC), HR+/HER2− breast cancer (HR+/HER2− BC), HR+/HER2low breast cancer (HR+/HER2low BC). In some embodiments, the cancer is pancreatic cancer. In some embodiments, the pancreatic cancer is pancreatic ductal adenocarcinoma (PDAC). In some embodiments, the cancer is metastatic. In some embodiments, the subject is human. In some embodiments, the subject is treatment naïve. In some embodiments, the subject has received at least one previous anticancer treatment selected from the group consisting of surgery, radiation therapy, chemotherapy, or checkpoint inhibitor therapy. In some embodiments, the cancer has progressed or recurred following an anticancer treatment selected from the group consisting of surgery, radiation therapy, chemotherapy, or checkpoint inhibitor therapy. In some embodiments, the cancer is resistant or refractory to checkpoint inhibitor therapy. In some embodiments, the checkpoint inhibitor therapy comprises an anti-PD-1 antibody or anti-PD-L1 antibody. In some embodiments, the anti-PD-1 antibody or anti-PD-L1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), MGD-019 (PD-1/CTLA4), KN-046 (PD-1/CTLA4), XmAb-20717 (PD-1/CTLA4), AK-104 (CTLA4/PD-1), MEDI-5752 (CTLA4/PD-1), atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, atezolizumab, avelumab, envafolimab, durvalumab, cosibelimab, lodapolimab, garivulimab, envafolimab, opucolimab, and manelimab. In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, and vopratelimab. In some embodiments, the chemotherapy is selected from the group consisting of 5-FU, leucovorin, oxaliplatin, irinotecan, gemcitabine, nab-paclitaxel (Abraxane®), FOLFIRINOX, and combinations thereof. In some embodiments, therapeutic agents used to treat pancreatic cancer comprise a combination of (a) 5-FU+leucovorin+oxaliplatin+irinotecan, (b) 5-FU+nanoliposomal irinotecan, (c) leucovorin+nanoliposomal irinotecan, or (d) gemcitabine+nab-paclitaxel. In some embodiments, the method further comprises co-administering an additional therapeutic agent or therapeutic modality.

In some embodiments, provided herein are methods for treating, mitigating, reducing, preventing or delaying the recurrence or metastasis of cancer in a subject comprising co-administering to the subject an effective amount of (a) an integrin activity modulator; and (b) a PD-1 inhibitor or PD-L1 inhibitor, wherein (i) the integrin activity modulator is selected from bexotegrast (i.e., PLN-74809), PLN-1474, and PLN-1561; or (ii) the PD-1 inhibitor or PD-L1 inhibitor is selected from atezolizumab, durvalumab, MK-7684A, nivolumab, pembrolizumab, and zimberelimab. In some embodiments, the integrin activity modulator is selected from an integrin-activating agent, an integrin-inhibiting agent, or an agent that is capable of interacting with one or more integrins. In some embodiments, the integrin activity modulator is a small molecule or a large molecule. In some embodiments, the large molecule is an antibody or antibody fragment. In some embodiments, the large molecule is an inhibitory peptide or peptide antagonist. In some embodiments, the integrin activity modulator activates, inhibits, or interacts with an integrin alpha (α) subunit. In some embodiments, the integrin a subunit is selected from α5, αV, α8, and αIIb. In some embodiments, the integrin activity modulator activates, inhibits, or interacts with the αV subunit. In some embodiments, the integrin activity modulator is an anti-αV antibody. In some embodiments, the anti-αV antibody is selected from abituzumab and intetumumab. In some embodiments, the integrin activity modulator is an iRGD peptide. In some embodiments, the iRGD peptide is CEND-1. In some embodiments, the integrin activity modulator activates, inhibits, or interacts with an integrin beta (β) subunit. In some embodiments, the β subunit is selected from β1, β3, β5, and β8. In some embodiments, the integrin activity modulator activates, inhibits, or interacts with β1. In some embodiments, the integrin activity modulator is an anti-β1 antibody. In some embodiments, the anti-β1 antibody is OS2966. In some embodiments, the integrin activity modulator activates, inhibits, or interacts with β5. In some embodiments, the integrin activity modulator is an anti-β5 antibody. In some embodiments, the anti-β5 antibody is ALULA. In some embodiments, the integrin activity modulator activates, inhibits, or interacts with an RGD-binding integrin. In some embodiments, RGD-binding integrin is selected from α5β1, αVβ1, αVβ3, αVβ5, αVβ6, αVβ8, α8β1, and αIIbβ3. In some embodiments, the integrin activity modulator activates, inhibits, or interacts with α5β1. In some embodiments, the integrin activity modulator is selected from AG-73305, AP25, AMEP, ATN-161, F-200 (Eos), JSM-6427, liposomal contortrostatin, PF-04605412, bexotegrast (i.e., PLN-74809), and SJ-749. In some embodiments, the integrin activity modulator activates, inhibits, or interacts with αVβ1. In some embodiments, the integrin activity modulator is selected from bexotegrast (i.e., PLN-74809), IDL-2965, PLN-1474, PLN-1561, and volociximab. In some embodiments, the integrin activity modulator is selected from bexotegrast (i.e., PLN-74809), PLN-1474, and PLN-1561. In some embodiments, the integrin activity modulator is bexotegrast. In some embodiments, the integrin activity modulator is PLN-1474. In some embodiments, the integrin activity modulator is PLN-1561. In some embodiments, the integrin activity modulator activates, inhibits, or interacts with αVβ3. In some embodiments, the integrin activity modulator is selected from AP25, B-1451, BS-1417, cilengitide, CT-6725, efatucizumab, etaracizumab, FF-10158, HM-3, IPS-02001, IPS-02101, IPS-05002, JNJ-26076713, MK-0429, NTA-1, NVX-188, Ocurin, ProAgio, PS-388023, RP-697, RP-748, SB-273005, Sch-221153, SC-69000, SD-983, SF-0166, SM-256, SOM-0777, SQ-885, TSRI-265, VIP-236, vitaxin, VPI-2690B, and XT-199. In some embodiments, the integrin activity modulator activates, inhibits, or interacts with αVβ5. In some embodiments, the integrin activity modulator is selected from ALULA, SNAKA-51, and STX-200. In some embodiments, the integrin activity modulator activates, inhibits, or interacts with αVβ6. In some embodiments, the integrin activity modulator is selected from 264-RAD, A20FMDV2, abituzumab, ADVCA0848, bexotegrast (i.e., PLN-74809), CRB-602, GSK-3008348, IDL-2965, intetumumab, MORF-627, MORF-720, PLN-1177, PLN-1561, PLN-1705, SGN-B6A, and STX-100. In some embodiments, the integrin activity modulator activates, inhibits, or interacts with αVβ8. In some embodiments, the integrin activity modulator is selected from CRB-601 and PF-06940434. In some embodiments, the integrin activity modulator activates, inhibits, or interacts with α8β1. In some embodiments, the integrin activity modulator is an anti-α8β1 antibody. In some embodiments, the anti-α8β1 antibody is an anti-α8β1 antibody disclosed in WO2011049082. In some embodiments, the integrin activity modulator activates, inhibits, or interacts with αIIbβ3. In some embodiments, the integrin activity modulator is selected from eptifibatide, RUC-1, Tc-99m-P280, tirofiban, zalunfiban, and an antibody that interacts with αIIbβ3. In some embodiments, the PD-1 inhibitor or PD-L1 inhibitor is an anti-PD-1 antibody. In some embodiments, the anti-PD-1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), PF-06801591, MGD-019 (PD-1/CTLA4), KN-046 (PD-1/CTLA4), XmAb-20717 (PD-1/CTLA4), AK-104 (CTLA4/PD-1), and MEDI-5752 (CTLA4/PD-1). In some embodiments, the anti-PD-1 antibody is zimberelimab. In some embodiments, the PD-1 inhibitor or PD-L1 inhibitor is an anti-PD-L1 antibody. In some embodiments, the PD-L1 inhibitor is selected from the group consisting of atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the PD-L1 inhibitor is a small molecule inhibitor. In some embodiments, the small molecule PD-L1 inhibitor is selected from the group consisting of CA-170, GS-4224, GS-4416, INCB99280, INCB99318, and lazertinib. In some embodiments, the PD-1 inhibitor or PD-L1 inhibitor is atezolizumab. In some embodiments, the PD-1 inhibitor or PD-L1 inhibitor is durvalumab. In some embodiments, the PD-1 inhibitor or PD-L1 inhibitor is MK-7684A. In some embodiments, the PD-1 inhibitor or PD-L1 inhibitor is nivolumab. In some embodiments, the PD-1 inhibitor or PD-L1 inhibitor is pembrolizumab. In some embodiments, the PD-1 inhibitor or PD-L1 inhibitor is zimberelimab. In some embodiments, the dose of the one or more integrin activity modulators is at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg. In some embodiments, the dose of the one or more integrin activity modulators is between about 5 mg to about 500 mg, about 10 mg to about 500 mg, about 15 mg to about 500 mg, about 20 mg to about 500 mg, about 25 mg to about 500 mg, about 30 mg to about 500 mg, about 35 mg to about 500 mg, about 40 mg to about 500 mg, about 45 mg to about 500 mg, about 50 mg to about 500 mg, about 55 mg to about 500 mg, about 60 mg to about 500 mg, about 65 mg to about 500 mg, about 70 mg to about 500 mg, about 75 mg to about 500 mg, about 80 mg to about 500 mg, about 85 mg to about 500 mg, about 90 mg to about 500 mg, about 95 mg to about 500 mg, about 100 mg to about 500 mg, about 110 mg to about 500 mg, about 120 mg to about 500 mg, about 130 mg to about 500 mg, about 140 mg to about 500 mg, about 150 mg to about 500 mg, about 160 mg to about 500 mg, about 170 mg to about 500 mg, about 180 mg to about 500 mg, about 190 mg to about 500 mg, about 200 mg to about 500 mg, about 210 mg to about 500 mg, about 220 mg to about 500 mg, about 230 mg to about 500 mg, about 240 mg to about 500 mg, about 250 mg to about 500 mg, about 260 mg to about 500 mg, about 270 mg to about 500 mg, about 280 mg to about 500 mg, about 290 mg to about 500 mg, or about 300 mg to about 500 mg. In some embodiments, the dose of the one or more integrin activity modulators is between about 5 mg to about 400 mg, about 10 mg to about 400 mg, about 15 mg to about 400 mg, about 20 mg to about 400 mg, about 25 mg to about 400 mg, about 30 mg to about 400 mg, about 35 mg to about 400 mg, about 40 mg to about 400 mg, about 45 mg to about 400 mg, about 50 mg to about 400 mg, about 55 mg to about 400 mg, about 60 mg to about 400 mg, about 65 mg to about 400 mg, about 70 mg to about 400 mg, about 75 mg to about 400 mg, about 80 mg to about 400 mg, about 85 mg to about 400 mg, about 90 mg to about 400 mg, about 95 mg to about 400 mg, about 100 mg to about 400 mg, about 110 mg to about 400 mg, about 120 mg to about 400 mg, about 130 mg to about 400 mg, about 140 mg to about 400 mg, about 150 mg to about 400 mg, about 160 mg to about 400 mg, about 170 mg to about 400 mg, about 180 mg to about 400 mg, about 190 mg to about 400 mg, about 200 mg to about 400 mg, about 210 mg to about 400 mg, about 220 mg to about 400 mg, about 230 mg to about 400 mg, about 240 mg to about 400 mg, about 250 mg to about 400 mg, about 260 mg to about 400 mg, about 270 mg to about 400 mg, about 280 mg to about 400 mg, about 290 mg to about 400 mg, or about 300 mg to about 400 mg. In some embodiments, the dose of the one or more integrin activity modulators is between about 5 mg to about 300 mg, about 10 mg to about 300 mg, about 15 mg to about 300 mg, about 20 mg to about 300 mg, about 25 mg to about 300 mg, about 30 mg to about 300 mg, about 35 mg to about 300 mg, about 40 mg to about 300 mg, about 45 mg to about 300 mg, about 50 mg to about 300 mg, about 55 mg to about 300 mg, about 60 mg to about 300 mg, about 65 mg to about 300 mg, about 70 mg to about 300 mg, about 75 mg to about 300 mg, about 80 mg to about 300 mg, about 85 mg to about 300 mg, about 90 mg to about 300 mg, about 95 mg to about 300 mg, about 100 mg to about 300 mg, about 110 mg to about 300 mg, about 120 mg to about 300 mg, about 130 mg to about 300 mg, about 140 mg to about 300 mg, about 150 mg to about 300 mg, about 160 mg to about 300 mg, about 170 mg to about 300 mg, about 180 mg to about 300 mg, about 190 mg to about 300 mg, about 200 mg to about 300 mg, about 210 mg to about 300 mg, about 220 mg to about 300 mg, about 230 mg to about 300 mg, about 240 mg to about 300 mg, about 250 mg to about 300 mg, about 260 mg to about 300 mg, about 270 mg to about 300 mg, about 280 mg to about 300 mg, or about 290 mg to about 300 mg. In some embodiments, the dose of the integrin activity modulator is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, the dose of the one or more integrin activity modulators is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the one or more integrin activity modulators is administered in at least 2, 3, 4, 5, 6, 7, 8, 9, 10, 11, 12, 13, 14, 15, 16, 17, 18, 19, or 20 or more doses. In some embodiments, the 2, 3, 4, 5, 6, 7, 8, 9, 10, 11, 12, 13, 14, 15, 16, 17, 18, 19, or 20 or more doses are the same. In some embodiments, the 2, 3, 4, 5, 6, 7, 8, 9, 10, 11, 12, 13, 14, 15, 16, 17, 18, 19, or 20 or more doses are different. In some embodiments, the integrin activity modulator is administered daily. In some embodiments, two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 days apart. In some embodiments, two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks apart. In some embodiments, two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 months apart. In some embodiments, the dose of bexotegrast is at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg. In some embodiments, the dose of bexotegrast is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the dose of bexotegrast is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, bexotegrast is administered daily. In some embodiments, bexotegrast is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. In some embodiments, bexotegrast is administered orally. In some embodiments, any of the methods or kits disclosed herein comprise one or more PD-1 or PD-L1 inhibitors. In some embodiments, the dose of the PD-1 inhibitor or PD-L1 inhibitor is at least about 30 mg, 60 mg, 120 mg, 240 mg, 360 mg, 480 mg, 600 mg, 720 mg, 840 mg, 960 mg, 1080 mg, or 1200 mg. In some embodiments, the dose of the PD-1 inhibitor or PD-L1 inhibitor is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the dose of the PD-1 inhibitor or PD-L1 inhibitor is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, the PD-1 inhibitor or PD-L1 inhibitor is administered daily. In some embodiments, two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 days apart. In some embodiments, two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks apart. In some embodiments, two or more doses of the PD-1 inhibitor or PD-L1 inhibitor are administered at least 3 weeks about. In some embodiments, two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 months apart. In some embodiments, the PD-1 inhibitor or PD-L1 inhibitor is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. In some embodiments, the dose of zimberelimab is at least about 30 mg, 60 mg, 120 mg, 240 mg, 360 mg, 480 mg, 600 mg, 720 mg, 840 mg, 960 mg, 1080 mg, or 1200 mg. In some embodiments, the dose of zimberelimab is at least about 360 mg. In some embodiments, the dose of zimberelimab is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the dose of zimberelimab is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, two or more doses of zimberelimab are administered at least about 1, 2, 3, 4, 5, 6, 7, 8, 9 10, 11, or 12 weeks apart. In some embodiments, two or more doses of zimberelimab are administered at least about every 3 weeks. In some embodiments, zimberelimab is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularily, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. In some embodiments, zimberelimab is administered intravenously. In some embodiments, zimberelimab is administered subcutaneously. In some embodiments, the cancer is a solid tumor. In some embodiments, the cancer is selected from breast cancer or pancreatic cancer. In some embodiments, the cancer is breast cancer. In some embodiments, the breast cancer is triple-negative breast cancer (TNBC), HR+/HER2− breast cancer (HR+/HER2− BC), HR+/HER2low breast cancer (HR+/HER2low BC). In some embodiments, the cancer is pancreatic cancer. In some embodiments, the pancreatic cancer is pancreatic ductal adenocarcinoma (PDAC). In some embodiments, the cancer is metastatic. In some embodiments, the subject is human. In some embodiments, the subject is treatment naïve. In some embodiments, the subject has received at least one previous anticancer treatment selected from the group consisting of surgery, radiation therapy, chemotherapy, or checkpoint inhibitor therapy. In some embodiments, the cancer has progressed or recurred following an anticancer treatment selected from the group consisting of surgery, radiation therapy, chemotherapy, or checkpoint inhibitor therapy. In some embodiments, the cancer is resistant or refractory to checkpoint inhibitor therapy. In some embodiments, the checkpoint inhibitor therapy comprises an anti-PD-1 antibody or anti-PD-L1 antibody. In some embodiments, the anti-PD-1 antibody or anti-PD-L1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), MGD-019 (PD-1/CTLA4), KN-046 (PD-1/CTLA4), XmAb-20717 (PD-1/CTLA4), AK-104 (CTLA4/PD-1), MEDI-5752 (CTLA4/PD-1), atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, atezolizumab, avelumab, envafolimab, durvalumab, cosibelimab, lodapolimab, garivulimab, envafolimab, opucolimab, and manelimab. In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, and vopratelimab. In some embodiments, the chemotherapy is selected from the group consisting of 5-FU, leucovorin, oxaliplatin, irinotecan, gemcitabine, nab-paclitaxel (Abraxane®), FOLFIRINOX, and combinations thereof. In some embodiments, therapeutic agents used to treat pancreatic cancer comprise a combination of (a) 5-FU+leucovorin+oxaliplatin+irinotecan, (b) 5-FU+nanoliposomal irinotecan, (c) leucovorin+nanoliposomal irinotecan, or (d) gemcitabine+nab-paclitaxel. In some embodiments, the method further comprises co-administering an additional therapeutic agent or therapeutic modality.

In some embodiments, provided herein are methods for eliciting an immune response to cancer in a subject comprising co-administering to the subject an effective amount of (a) an integrin activity modulator; and (b) a PD-1 inhibitor or PD-L1 inhibitor, wherein (i) the integrin activity modulator is selected from bexotegrast (i.e., PLN-74809), PLN-1474, and PLN-1561; or (ii) the PD-1 inhibitor or PD-L1 inhibitor is selected from atezolizumab, durvalumab, MK-7684A, nivolumab, pembrolizumab, and zimberelimab. In some embodiments, the integrin activity modulator is selected from an integrin-activating agent, an integrin-inhibiting agent, or an agent that is capable of interacting with one or more integrins. In some embodiments, the integrin activity modulator is a small molecule or a large molecule. In some embodiments, the large molecule is an antibody or antibody fragment. In some embodiments, the large molecule is an inhibitory peptide or peptide antagonist. In some embodiments, the integrin activity modulator activates, inhibits, or interacts with an integrin alpha (α) subunit. In some embodiments, the integrin a subunit is selected from α5, αV, α8, and αIIb. In some embodiments, the integrin activity modulator activates, inhibits, or interacts with the αV subunit. In some embodiments, the integrin activity modulator is an anti-αV antibody. In some embodiments, the anti-αV antibody is selected from abituzumab and intetumumab. In some embodiments, the integrin activity modulator is an iRGD peptide. In some embodiments, the iRGD peptide is CEND-1. In some embodiments, the integrin activity modulator activates, inhibits, or interacts with an integrin beta (β) subunit. In some embodiments, the β subunit is selected from β1, β3, β5, and β8. In some embodiments, the integrin activity modulator activates, inhibits, or interacts with β1. In some embodiments, the integrin activity modulator is an anti-β1 antibody. In some embodiments, the anti-β1 antibody is OS2966. In some embodiments, the integrin activity modulator activates, inhibits, or interacts with β5. In some embodiments, the integrin activity modulator is an anti-β5 antibody. In some embodiments, the anti-β5 antibody is ALULA. In some embodiments, the integrin activity modulator activates, inhibits, or interacts with an RGD-binding integrin. In some embodiments, RGD-binding integrin is selected from α5β1, αVβ1, αVβ3, αVβ5, αVβ6, αVβ8, α8β1, and αIIbβ3. In some embodiments, the integrin activity modulator activates, inhibits, or interacts with α5β1. In some embodiments, the integrin activity modulator is selected from AG-73305, AP25, AMEP, ATN-161, F-200 (Eos), JSM-6427, liposomal contortrostatin, PF-04605412, bexotegrast (i.e., PLN-74809), and SJ-749. In some embodiments, the integrin activity modulator activates, inhibits, or interacts with αVβ1. In some embodiments, the integrin activity modulator is selected from bexotegrast (i.e., PLN-74809), IDL-2965, PLN-1474, PLN-1561, and volociximab. In some embodiments, the integrin activity modulator is selected from bexotegrast (i.e., PLN-74809), PLN-1474, and PLN-1561. In some embodiments, the integrin activity modulator is bexotegrast. In some embodiments, the integrin activity modulator is PLN-1474. In some embodiments, the integrin activity modulator is PLN-1561. In some embodiments, the integrin activity modulator activates, inhibits, or interacts with αVβ3. In some embodiments, the integrin activity modulator is selected from AP25, B-1451, BS-1417, cilengitide, CT-6725, efatucizumab, etaracizumab, FF-10158, HM-3, IPS-02001, IPS-02101, IPS-05002, JNJ-26076713, MK-0429, NTA-1, NVX-188, Ocurin, ProAgio, PS-388023, RP-697, RP-748, SB-273005, Sch-221153, SC-69000, SD-983, SF-0166, SM-256, SOM-0777, SQ-885, TSRI-265, VIP-236, vitaxin, VPI-2690B, and XT-199. In some embodiments, the integrin activity modulator activates, inhibits, or interacts with αVβ5. In some embodiments, the integrin activity modulator is selected from ALULA, SNAKA-51, and STX-200. In some embodiments, the integrin activity modulator activates, inhibits, or interacts with αVβ6. In some embodiments, the integrin activity modulator is selected from 264-RAD, A20FMDV2, abituzumab, ADVCA0848, bexotegrast (i.e., PLN-74809), CRB-602, GSK-3008348, IDL-2965, intetumumab, MORF-627, MORF-720, PLN-1177, PLN-1561, PLN-1705, SGN-B6A, and STX-100. In some embodiments, the integrin activity modulator activates, inhibits, or interacts with αVβ8. In some embodiments, the integrin activity modulator is selected from CRB-601 and PF-06940434. In some embodiments, the integrin activity modulator activates, inhibits, or interacts with α8β1. In some embodiments, the integrin activity modulator is an anti-α8β1 antibody. In some embodiments, the anti-α8β1 antibody is an anti-α8β1 antibody disclosed in WO2011049082. In some embodiments, the integrin activity modulator activates, inhibits, or interacts with αIIbβ3. In some embodiments, the integrin activity modulator is selected from eptifibatide, RUC-1, Tc-99m-P280, tirofiban, zalunfiban, and an antibody that interacts with αIIbβ3. In some embodiments, the PD-1 inhibitor or PD-L1 inhibitor is an anti-PD-1 antibody. In some embodiments, the anti-PD-1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), PF-06801591, MGD-019 (PD-1/CTLA4), KN-046 (PD-1/CTLA4), XmAb-20717 (PD-1/CTLA4), AK-104 (CTLA4/PD-1), and MEDI-5752 (CTLA4/PD-1). In some embodiments, the anti-PD-1 antibody is zimberelimab. In some embodiments, the PD-1 inhibitor or PD-L1 inhibitor is an anti-PD-L1 antibody. In some embodiments, the PD-L1 inhibitor is selected from the group consisting of atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the PD-L1 inhibitor is a small molecule inhibitor. In some embodiments, the small molecule PD-L1 inhibitor is selected from the group consisting of CA-170, GS-4224, GS-4416, INCB99280, INCB99318, and lazertinib. In some embodiments, the PD-1 inhibitor or PD-L1 inhibitor is atezolizumab. In some embodiments, the PD-1 inhibitor or PD-L1 inhibitor is durvalumab. In some embodiments, the PD-1 inhibitor or PD-L1 inhibitor is MK-7684A. In some embodiments, the PD-1 inhibitor or PD-L1 inhibitor is nivolumab. In some embodiments, the PD-1 inhibitor or PD-L1 inhibitor is pembrolizumab. In some embodiments, the PD-1 inhibitor or PD-L1 inhibitor is zimberelimab. In some embodiments, the dose of the one or more integrin activity modulators is at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg. In some embodiments, the dose of the one or more integrin activity modulators is between about 5 mg to about 500 mg, about 10 mg to about 500 mg, about 15 mg to about 500 mg, about 20 mg to about 500 mg, about 25 mg to about 500 mg, about 30 mg to about 500 mg, about 35 mg to about 500 mg, about 40 mg to about 500 mg, about 45 mg to about 500 mg, about 50 mg to about 500 mg, about 55 mg to about 500 mg, about 60 mg to about 500 mg, about 65 mg to about 500 mg, about 70 mg to about 500 mg, about 75 mg to about 500 mg, about 80 mg to about 500 mg, about 85 mg to about 500 mg, about 90 mg to about 500 mg, about 95 mg to about 500 mg, about 100 mg to about 500 mg, about 110 mg to about 500 mg, about 120 mg to about 500 mg, about 130 mg to about 500 mg, about 140 mg to about 500 mg, about 150 mg to about 500 mg, about 160 mg to about 500 mg, about 170 mg to about 500 mg, about 180 mg to about 500 mg, about 190 mg to about 500 mg, about 200 mg to about 500 mg, about 210 mg to about 500 mg, about 220 mg to about 500 mg, about 230 mg to about 500 mg, about 240 mg to about 500 mg, about 250 mg to about 500 mg, about 260 mg to about 500 mg, about 270 mg to about 500 mg, about 280 mg to about 500 mg, about 290 mg to about 500 mg, or about 300 mg to about 500 mg. In some embodiments, the dose of the one or more integrin activity modulators is between about 5 mg to about 400 mg, about 10 mg to about 400 mg, about 15 mg to about 400 mg, about 20 mg to about 400 mg, about 25 mg to about 400 mg, about 30 mg to about 400 mg, about 35 mg to about 400 mg, about 40 mg to about 400 mg, about 45 mg to about 400 mg, about 50 mg to about 400 mg, about 55 mg to about 400 mg, about 60 mg to about 400 mg, about 65 mg to about 400 mg, about 70 mg to about 400 mg, about 75 mg to about 400 mg, about 80 mg to about 400 mg, about 85 mg to about 400 mg, about 90 mg to about 400 mg, about 95 mg to about 400 mg, about 100 mg to about 400 mg, about 110 mg to about 400 mg, about 120 mg to about 400 mg, about 130 mg to about 400 mg, about 140 mg to about 400 mg, about 150 mg to about 400 mg, about 160 mg to about 400 mg, about 170 mg to about 400 mg, about 180 mg to about 400 mg, about 190 mg to about 400 mg, about 200 mg to about 400 mg, about 210 mg to about 400 mg, about 220 mg to about 400 mg, about 230 mg to about 400 mg, about 240 mg to about 400 mg, about 250 mg to about 400 mg, about 260 mg to about 400 mg, about 270 mg to about 400 mg, about 280 mg to about 400 mg, about 290 mg to about 400 mg, or about 300 mg to about 400 mg. In some embodiments, the dose of the one or more integrin activity modulators is between about 5 mg to about 300 mg, about 10 mg to about 300 mg, about 15 mg to about 300 mg, about 20 mg to about 300 mg, about 25 mg to about 300 mg, about 30 mg to about 300 mg, about 35 mg to about 300 mg, about 40 mg to about 300 mg, about 45 mg to about 300 mg, about 50 mg to about 300 mg, about 55 mg to about 300 mg, about 60 mg to about 300 mg, about 65 mg to about 300 mg, about 70 mg to about 300 mg, about 75 mg to about 300 mg, about 80 mg to about 300 mg, about 85 mg to about 300 mg, about 90 mg to about 300 mg, about 95 mg to about 300 mg, about 100 mg to about 300 mg, about 110 mg to about 300 mg, about 120 mg to about 300 mg, about 130 mg to about 300 mg, about 140 mg to about 300 mg, about 150 mg to about 300 mg, about 160 mg to about 300 mg, about 170 mg to about 300 mg, about 180 mg to about 300 mg, about 190 mg to about 300 mg, about 200 mg to about 300 mg, about 210 mg to about 300 mg, about 220 mg to about 300 mg, about 230 mg to about 300 mg, about 240 mg to about 300 mg, about 250 mg to about 300 mg, about 260 mg to about 300 mg, about 270 mg to about 300 mg, about 280 mg to about 300 mg, or about 290 mg to about 300 mg. In some embodiments, the dose of the integrin activity modulator is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, the dose of the one or more integrin activity modulators is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the one or more integrin activity modulators is administered in at least 2, 3, 4, 5, 6, 7, 8, 9, 10, 11, 12, 13, 14, 15, 16, 17, 18, 19, or 20 or more doses. In some embodiments, the 2, 3, 4, 5, 6, 7, 8, 9, 10, 11, 12, 13, 14, 15, 16, 17, 18, 19, or 20 or more doses are the same. In some embodiments, the 2, 3, 4, 5, 6, 7, 8, 9, 10, 11, 12, 13, 14, 15, 16, 17, 18, 19, or 20 or more doses are different. In some embodiments, the integrin activity modulator is administered daily. In some embodiments, two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 days apart. In some embodiments, two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks apart. In some embodiments, two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 months apart. In some embodiments, the dose of bexotegrast is at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg. In some embodiments, the dose of bexotegrast is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the dose of bexotegrast is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, bexotegrast is administered daily. In some embodiments, bexotegrast is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. In some embodiments, bexotegrast is administered orally. In some embodiments, any of the methods or kits disclosed herein comprise one or more PD-1 or PD-L1 inhibitors. In some embodiments, the dose of the PD-1 inhibitor or PD-L1 inhibitor is at least about 30 mg, 60 mg, 120 mg, 240 mg, 360 mg, 480 mg, 600 mg, 720 mg, 840 mg, 960 mg, 1080 mg, or 1200 mg. In some embodiments, the dose of the PD-1 inhibitor or PD-L1 inhibitor is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the dose of the PD-1 inhibitor or PD-L1 inhibitor is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, the PD-1 inhibitor or PD-L1 inhibitor is administered daily. In some embodiments, two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 days apart. In some embodiments, two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks apart. In some embodiments, two or more doses of the PD-1 inhibitor or PD-L1 inhibitor are administered at least 3 weeks about. In some embodiments, two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 months apart. In some embodiments, the PD-1 inhibitor or PD-L1 inhibitor is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularily, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. In some embodiments, the dose of zimberelimab is at least about 30 mg, 60 mg, 120 mg, 240 mg, 360 mg, 480 mg, 600 mg, 720 mg, 840 mg, 960 mg, 1080 mg, or 1200 mg. In some embodiments, the dose of zimberelimab is at least about 360 mg. In some embodiments, the dose of zimberelimab is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the dose of zimberelimab is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, two or more doses of zimberelimab are administered at least about 1, 2, 3, 4, 5, 6, 7, 8, 9 10, 11, or 12 weeks apart. In some embodiments, two or more doses of zimberelimab are administered at least about every 3 weeks. In some embodiments, zimberelimab is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. In some embodiments, zimberelimab is administered intravenously. In some embodiments, zimberelimab is administered subcutaneously. In some embodiments, the cancer is a solid tumor. In some embodiments, the cancer is selected from breast cancer or pancreatic cancer. In some embodiments, the cancer is breast cancer. In some embodiments, the breast cancer is triple-negative breast cancer (TNBC), HR+/HER2− breast cancer (HR+/HER2− BC), HR+/HER2low breast cancer (HR+/HER2low BC). In some embodiments, the cancer is pancreatic cancer. In some embodiments, the pancreatic cancer is pancreatic ductal adenocarcinoma (PDAC). In some embodiments, the cancer is metastatic. In some embodiments, the subject is human. In some embodiments, the subject is treatment naïve. In some embodiments, the subject has received at least one previous anticancer treatment selected from the group consisting of surgery, radiation therapy, chemotherapy, or checkpoint inhibitor therapy. In some embodiments, the cancer has progressed or recurred following an anticancer treatment selected from the group consisting of surgery, radiation therapy, chemotherapy, or checkpoint inhibitor therapy. In some embodiments, the cancer is resistant or refractory to checkpoint inhibitor therapy. In some embodiments, the checkpoint inhibitor therapy comprises an anti-PD-1 antibody or anti-PD-L1 antibody. In some embodiments, the anti-PD-1 antibody or anti-PD-L1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), MGD-019 (PD-1/CTLA4), KN-046 (PD-1/CTLA4), XmAb-20717 (PD-1/CTLA4), AK-104 (CTLA4/PD-1), MEDI-5752 (CTLA4/PD-1), atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, atezolizumab, avelumab, envafolimab, durvalumab, cosibelimab, lodapolimab, garivulimab, envafolimab, opucolimab, and manelimab. In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, and vopratelimab. In some embodiments, the chemotherapy is selected from the group consisting of 5-FU, leucovorin, oxaliplatin, irinotecan, gemcitabine, nab-paclitaxel (Abraxane®), FOLFIRINOX, and combinations thereof. In some embodiments, therapeutic agents used to treat pancreatic cancer comprise a combination of (a) 5-FU+leucovorin+oxaliplatin+irinotecan, (b) 5-FU+nanoliposomal irinotecan, (c) leucovorin+nanoliposomal irinotecan, or (d) gemcitabine+nab-paclitaxel. In some embodiments, the method further comprises co-administering an additional therapeutic agent or therapeutic modality.

In some embodiments, provided herein are methods treating, mitigating, reducing, preventing or delaying the recurrence or metastasis of pancreatic cancer or breast cancer in a subject comprising co-administering to the subject an effective amount of (a) an integrin activity modulator selected from bexotegrast (i.e., PLN-74809), PLN-1474, and PLN-1561; and (b) a PD-1 inhibitor or PD-L1 inhibitor selected from atezolizumab, durvalumab, MK-7684A, nivolumab, pembrolizumab, and zimberelimab. In some embodiments, the integrin activity modulator is bexotegrast. In some embodiments, the integrin activity modulator is PLN-1474. In some embodiments, the integrin activity modulator is PLN-1561. In some embodiments, the PD-1 inhibitor or PD-L1 inhibitor is atezolizumab. In some embodiments, the PD-1 inhibitor or PD-L1 inhibitor is durvalumab. In some embodiments, the PD-1 inhibitor or PD-L1 inhibitor is MK-7684A. In some embodiments, the PD-1 inhibitor or PD-L1 inhibitor is nivolumab. In some embodiments, the PD-1 inhibitor or PD-L1 inhibitor is pembrolizumab. In some embodiments, the PD-1 inhibitor or PD-L1 inhibitor is zimberelimab. In some embodiments, the dose of the one or more integrin activity modulators is at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg. In some embodiments, the dose of the one or more integrin activity modulators is between about 5 mg to about 500 mg, about 10 mg to about 500 mg, about 15 mg to about 500 mg, about 20 mg to about 500 mg, about 25 mg to about 500 mg, about 30 mg to about 500 mg, about 35 mg to about 500 mg, about 40 mg to about 500 mg, about 45 mg to about 500 mg, about 50 mg to about 500 mg, about 55 mg to about 500 mg, about 60 mg to about 500 mg, about 65 mg to about 500 mg, about 70 mg to about 500 mg, about 75 mg to about 500 mg, about 80 mg to about 500 mg, about 85 mg to about 500 mg, about 90 mg to about 500 mg, about 95 mg to about 500 mg, about 100 mg to about 500 mg, about 110 mg to about 500 mg, about 120 mg to about 500 mg, about 130 mg to about 500 mg, about 140 mg to about 500 mg, about 150 mg to about 500 mg, about 160 mg to about 500 mg, about 170 mg to about 500 mg, about 180 mg to about 500 mg, about 190 mg to about 500 mg, about 200 mg to about 500 mg, about 210 mg to about 500 mg, about 220 mg to about 500 mg, about 230 mg to about 500 mg, about 240 mg to about 500 mg, about 250 mg to about 500 mg, about 260 mg to about 500 mg, about 270 mg to about 500 mg, about 280 mg to about 500 mg, about 290 mg to about 500 mg, or about 300 mg to about 500 mg. In some embodiments, the dose of the one or more integrin activity modulators is between about 5 mg to about 400 mg, about 10 mg to about 400 mg, about 15 mg to about 400 mg, about 20 mg to about 400 mg, about 25 mg to about 400 mg, about 30 mg to about 400 mg, about 35 mg to about 400 mg, about 40 mg to about 400 mg, about 45 mg to about 400 mg, about 50 mg to about 400 mg, about 55 mg to about 400 mg, about 60 mg to about 400 mg, about 65 mg to about 400 mg, about 70 mg to about 400 mg, about 75 mg to about 400 mg, about 80 mg to about 400 mg, about 85 mg to about 400 mg, about 90 mg to about 400 mg, about 95 mg to about 400 mg, about 100 mg to about 400 mg, about 110 mg to about 400 mg, about 120 mg to about 400 mg, about 130 mg to about 400 mg, about 140 mg to about 400 mg, about 150 mg to about 400 mg, about 160 mg to about 400 mg, about 170 mg to about 400 mg, about 180 mg to about 400 mg, about 190 mg to about 400 mg, about 200 mg to about 400 mg, about 210 mg to about 400 mg, about 220 mg to about 400 mg, about 230 mg to about 400 mg, about 240 mg to about 400 mg, about 250 mg to about 400 mg, about 260 mg to about 400 mg, about 270 mg to about 400 mg, about 280 mg to about 400 mg, about 290 mg to about 400 mg, or about 300 mg to about 400 mg. In some embodiments, the dose of the one or more integrin activity modulators is between about 5 mg to about 300 mg, about 10 mg to about 300 mg, about 15 mg to about 300 mg, about 20 mg to about 300 mg, about 25 mg to about 300 mg, about 30 mg to about 300 mg, about 35 mg to about 300 mg, about 40 mg to about 300 mg, about 45 mg to about 300 mg, about 50 mg to about 300 mg, about 55 mg to about 300 mg, about 60 mg to about 300 mg, about 65 mg to about 300 mg, about 70 mg to about 300 mg, about 75 mg to about 300 mg, about 80 mg to about 300 mg, about 85 mg to about 300 mg, about 90 mg to about 300 mg, about 95 mg to about 300 mg, about 100 mg to about 300 mg, about 110 mg to about 300 mg, about 120 mg to about 300 mg, about 130 mg to about 300 mg, about 140 mg to about 300 mg, about 150 mg to about 300 mg, about 160 mg to about 300 mg, about 170 mg to about 300 mg, about 180 mg to about 300 mg, about 190 mg to about 300 mg, about 200 mg to about 300 mg, about 210 mg to about 300 mg, about 220 mg to about 300 mg, about 230 mg to about 300 mg, about 240 mg to about 300 mg, about 250 mg to about 300 mg, about 260 mg to about 300 mg, about 270 mg to about 300 mg, about 280 mg to about 300 mg, or about 290 mg to about 300 mg. In some embodiments, the dose of the integrin activity modulator is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, the dose of the one or more integrin activity modulators is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the one or more integrin activity modulators is administered in at least 2, 3, 4, 5, 6, 7, 8, 9, 10, 11, 12, 13, 14, 15, 16, 17, 18, 19, or 20 or more doses. In some embodiments, the 2, 3, 4, 5, 6, 7, 8, 9, 10, 11, 12, 13, 14, 15, 16, 17, 18, 19, or 20 or more doses are the same. In some embodiments, the 2, 3, 4, 5, 6, 7, 8, 9, 10, 11, 12, 13, 14, 15, 16, 17, 18, 19, or 20 or more doses are different. In some embodiments, the integrin activity modulator is administered daily. In some embodiments, two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 days apart. In some embodiments, two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks apart. In some embodiments, two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 months apart. In some embodiments, the dose of bexotegrast is at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg. In some embodiments, the dose of bexotegrast is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the dose of bexotegrast is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, bexotegrast is administered daily. In some embodiments, bexotegrast is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. In some embodiments, bexotegrast is administered orally. In some embodiments, any of the methods or kits disclosed herein comprise one or more PD-1 or PD-L1 inhibitors. In some embodiments, the dose of the PD-1 inhibitor or PD-L1 inhibitor is at least about 30 mg, 60 mg, 120 mg, 240 mg, 360 mg, 480 mg, 600 mg, 720 mg, 840 mg, 960 mg, 1080 mg, or 1200 mg. In some embodiments, the dose of the PD-1 inhibitor or PD-L1 inhibitor is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the dose of the PD-1 inhibitor or PD-L1 inhibitor is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, the PD-1 inhibitor or PD-L1 inhibitor is administered daily. In some embodiments, two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 days apart. In some embodiments, two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks apart. In some embodiments, two or more doses of the PD-1 inhibitor or PD-L1 inhibitor are administered at least 3 weeks about. In some embodiments, two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 months apart. In some embodiments, the PD-1 inhibitor or PD-L1 inhibitor is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularily, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. In some embodiments, the dose of zimberelimab is at least about 30 mg, 60 mg, 120 mg, 240 mg, 360 mg, 480 mg, 600 mg, 720 mg, 840 mg, 960 mg, 1080 mg, or 1200 mg. In some embodiments, the dose of zimberelimab is at least about 360 mg. In some embodiments, the dose of zimberelimab is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the dose of zimberelimab is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, two or more doses of zimberelimab are administered at least about 1, 2, 3, 4, 5, 6, 7, 8, 9 10, 11, or 12 weeks apart. In some embodiments, two or more doses of zimberelimab are administered at least about every 3 weeks. In some embodiments, zimberelimab is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. In some embodiments, zimberelimab is administered intravenously. In some embodiments, zimberelimab is administered subcutaneously. In some embodiments, the breast cancer is triple-negative breast cancer (TNBC), HR+/HER2− breast cancer (HR+/HER2− BC), HR+/HER2low breast cancer (HR+/HER2low BC). In some embodiments, the pancreatic cancer is pancreatic ductal adenocarcinoma (PDAC). In some embodiments, the cancer is metastatic. In some embodiments, the subject is human. In some embodiments, the subject is treatment naïve. In some embodiments, the subject has received at least one previous anticancer treatment selected from the group consisting of surgery, radiation therapy, chemotherapy, or checkpoint inhibitor therapy. In some embodiments, the cancer has progressed or recurred following an anticancer treatment selected from the group consisting of surgery, radiation therapy, chemotherapy, or checkpoint inhibitor therapy. In some embodiments, the cancer is resistant or refractory to checkpoint inhibitor therapy. In some embodiments, the checkpoint inhibitor therapy comprises an anti-PD-1 antibody or anti-PD-L1 antibody. In some embodiments, the anti-PD-1 antibody or anti-PD-L1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), MGD-019 (PD-1/CTLA4), KN-046 (PD-1/CTLA4), XmAb-20717 (PD-1/CTLA4), AK-104 (CTLA4/PD-1), MEDI-5752 (CTLA4/PD-1), atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, atezolizumab, avelumab, envafolimab, durvalumab, cosibelimab, lodapolimab, garivulimab, envafolimab, opucolimab, and manelimab. In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, and vopratelimab. In some embodiments, the chemotherapy is selected from the group consisting of 5-FU, leucovorin, oxaliplatin, irinotecan, gemcitabine, nab-paclitaxel (Abraxane®), FOLFIRINOX, and combinations thereof. In some embodiments, therapeutic agents used to treat pancreatic cancer comprise a combination of (a) 5-FU+leucovorin+oxaliplatin+irinotecan, (b) 5-FU+nanoliposomal irinotecan, (c) leucovorin+nanoliposomal irinotecan, or (d) gemcitabine+nab-paclitaxel. In some embodiments, the method further comprises co-administering an additional therapeutic agent or therapeutic modality.

In some embodiments, provided herein are methods eliciting an immune response to pancreatic cancer or breast cancer in a subject comprising co-administering to the subject an effective amount of (a) an integrin activity modulator selected from bexotegrast (i.e., PLN-74809), PLN-1474, and PLN-1561; and (b) a PD-1 inhibitor or PD-L1 inhibitor selected from atezolizumab, durvalumab, MK-7684A, nivolumab, pembrolizumab, and zimberelimab. In some embodiments, the integrin activity modulator is bexotegrast. In some embodiments, the integrin activity modulator is PLN-1474. In some embodiments, the integrin activity modulator is PLN-1561. In some embodiments, the PD-1 inhibitor or PD-L1 inhibitor is atezolizumab. In some embodiments, the PD-1 inhibitor or PD-L1 inhibitor is durvalumab. In some embodiments, the PD-1 inhibitor or PD-L1 inhibitor is MK-7684A. In some embodiments, the PD-1 inhibitor or PD-L1 inhibitor is nivolumab. In some embodiments, the PD-1 inhibitor or PD-L1 inhibitor is pembrolizumab. In some embodiments, the PD-1 inhibitor or PD-L1 inhibitor is zimberelimab. In some embodiments, the dose of the one or more integrin activity modulators is at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg. In some embodiments, the dose of the one or more integrin activity modulators is between about 5 mg to about 500 mg, about 10 mg to about 500 mg, about 15 mg to about 500 mg, about 20 mg to about 500 mg, about 25 mg to about 500 mg, about 30 mg to about 500 mg, about 35 mg to about 500 mg, about 40 mg to about 500 mg, about 45 mg to about 500 mg, about 50 mg to about 500 mg, about 55 mg to about 500 mg, about 60 mg to about 500 mg, about 65 mg to about 500 mg, about 70 mg to about 500 mg, about 75 mg to about 500 mg, about 80 mg to about 500 mg, about 85 mg to about 500 mg, about 90 mg to about 500 mg, about 95 mg to about 500 mg, about 100 mg to about 500 mg, about 110 mg to about 500 mg, about 120 mg to about 500 mg, about 130 mg to about 500 mg, about 140 mg to about 500 mg, about 150 mg to about 500 mg, about 160 mg to about 500 mg, about 170 mg to about 500 mg, about 180 mg to about 500 mg, about 190 mg to about 500 mg, about 200 mg to about 500 mg, about 210 mg to about 500 mg, about 220 mg to about 500 mg, about 230 mg to about 500 mg, about 240 mg to about 500 mg, about 250 mg to about 500 mg, about 260 mg to about 500 mg, about 270 mg to about 500 mg, about 280 mg to about 500 mg, about 290 mg to about 500 mg, or about 300 mg to about 500 mg. In some embodiments, the dose of the one or more integrin activity modulators is between about 5 mg to about 400 mg, about 10 mg to about 400 mg, about 15 mg to about 400 mg, about 20 mg to about 400 mg, about 25 mg to about 400 mg, about 30 mg to about 400 mg, about 35 mg to about 400 mg, about 40 mg to about 400 mg, about 45 mg to about 400 mg, about 50 mg to about 400 mg, about 55 mg to about 400 mg, about 60 mg to about 400 mg, about 65 mg to about 400 mg, about 70 mg to about 400 mg, about 75 mg to about 400 mg, about 80 mg to about 400 mg, about 85 mg to about 400 mg, about 90 mg to about 400 mg, about 95 mg to about 400 mg, about 100 mg to about 400 mg, about 110 mg to about 400 mg, about 120 mg to about 400 mg, about 130 mg to about 400 mg, about 140 mg to about 400 mg, about 150 mg to about 400 mg, about 160 mg to about 400 mg, about 170 mg to about 400 mg, about 180 mg to about 400 mg, about 190 mg to about 400 mg, about 200 mg to about 400 mg, about 210 mg to about 400 mg, about 220 mg to about 400 mg, about 230 mg to about 400 mg, about 240 mg to about 400 mg, about 250 mg to about 400 mg, about 260 mg to about 400 mg, about 270 mg to about 400 mg, about 280 mg to about 400 mg, about 290 mg to about 400 mg, or about 300 mg to about 400 mg. In some embodiments, the dose of the one or more integrin activity modulators is between about 5 mg to about 300 mg, about 10 mg to about 300 mg, about 15 mg to about 300 mg, about 20 mg to about 300 mg, about 25 mg to about 300 mg, about 30 mg to about 300 mg, about 35 mg to about 300 mg, about 40 mg to about 300 mg, about 45 mg to about 300 mg, about 50 mg to about 300 mg, about 55 mg to about 300 mg, about 60 mg to about 300 mg, about 65 mg to about 300 mg, about 70 mg to about 300 mg, about 75 mg to about 300 mg, about 80 mg to about 300 mg, about 85 mg to about 300 mg, about 90 mg to about 300 mg, about 95 mg to about 300 mg, about 100 mg to about 300 mg, about 110 mg to about 300 mg, about 120 mg to about 300 mg, about 130 mg to about 300 mg, about 140 mg to about 300 mg, about 150 mg to about 300 mg, about 160 mg to about 300 mg, about 170 mg to about 300 mg, about 180 mg to about 300 mg, about 190 mg to about 300 mg, about 200 mg to about 300 mg, about 210 mg to about 300 mg, about 220 mg to about 300 mg, about 230 mg to about 300 mg, about 240 mg to about 300 mg, about 250 mg to about 300 mg, about 260 mg to about 300 mg, about 270 mg to about 300 mg, about 280 mg to about 300 mg, or about 290 mg to about 300 mg. In some embodiments, the dose of the integrin activity modulator is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, the dose of the one or more integrin activity modulators is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the one or more integrin activity modulators is administered in at least 2, 3, 4, 5, 6, 7, 8, 9, 10, 11, 12, 13, 14, 15, 16, 17, 18, 19, or 20 or more doses. In some embodiments, the 2, 3, 4, 5, 6, 7, 8, 9, 10, 11, 12, 13, 14, 15, 16, 17, 18, 19, or 20 or more doses are the same. In some embodiments, the 2, 3, 4, 5, 6, 7, 8, 9, 10, 11, 12, 13, 14, 15, 16, 17, 18, 19, or 20 or more doses are different. In some embodiments, the integrin activity modulator is administered daily. In some embodiments, two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 days apart. In some embodiments, two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks apart. In some embodiments, two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 months apart. In some embodiments, the dose of bexotegrast is at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg. In some embodiments, the dose of bexotegrast is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the dose of bexotegrast is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, bexotegrast is administered daily. In some embodiments, bexotegrast is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. In some embodiments, bexotegrast is administered orally. In some embodiments, any of the methods or kits disclosed herein comprise one or more PD-1 or PD-L1 inhibitors. In some embodiments, the dose of the PD-1 inhibitor or PD-L1 inhibitor is at least about 30 mg, 60 mg, 120 mg, 240 mg, 360 mg, 480 mg, 600 mg, 720 mg, 840 mg, 960 mg, 1080 mg, or 1200 mg. In some embodiments, the dose of the PD-1 inhibitor or PD-L1 inhibitor is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the dose of the PD-1 inhibitor or PD-L1 inhibitor is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, the PD-1 inhibitor or PD-L1 inhibitor is administered daily. In some embodiments, two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 days apart. In some embodiments, two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks apart. In some embodiments, two or more doses of the PD-1 inhibitor or PD-L1 inhibitor are administered at least 3 weeks about. In some embodiments, two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 months apart. In some embodiments, the PD-1 inhibitor or PD-L1 inhibitor is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularily, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. In some embodiments, the dose of zimberelimab is at least about 30 mg, 60 mg, 120 mg, 240 mg, 360 mg, 480 mg, 600 mg, 720 mg, 840 mg, 960 mg, 1080 mg, or 1200 mg. In some embodiments, the dose of zimberelimab is at least about 360 mg. In some embodiments, the dose of zimberelimab is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg. In some embodiments, the dose of zimberelimab is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg. In some embodiments, two or more doses of zimberelimab are administered at least about 1, 2, 3, 4, 5, 6, 7, 8, 9 10, 11, or 12 weeks apart. In some embodiments, two or more doses of zimberelimab are administered at least about every 3 weeks. In some embodiments, zimberelimab is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularily, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. In some embodiments, zimberelimab is administered intravenously. In some embodiments, zimberelimab is administered subcutaneously. In some embodiments, the breast cancer is triple-negative breast cancer (TNBC), HR+/HER2− breast cancer (HR+/HER2− BC), HR+/HER2low breast cancer (HR+/HER2low BC). In some embodiments, the pancreatic cancer is pancreatic ductal adenocarcinoma (PDAC). In some embodiments, the cancer is metastatic. In some embodiments, the subject is human. In some embodiments, the subject is treatment naïve. In some embodiments, the subject has received at least one previous anticancer treatment selected from the group consisting of surgery, radiation therapy, chemotherapy, or checkpoint inhibitor therapy. In some embodiments, the cancer has progressed or recurred following an anticancer treatment selected from the group consisting of surgery, radiation therapy, chemotherapy, or checkpoint inhibitor therapy. In some embodiments, the cancer is resistant or refractory to checkpoint inhibitor therapy. In some embodiments, the checkpoint inhibitor therapy comprises an anti-PD-1 antibody or anti-PD-L1 antibody. In some embodiments, the anti-PD-1 antibody or anti-PD-L1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), MGD-019 (PD-1/CTLA4), KN-046 (PD-1/CTLA4), XmAb-20717 (PD-1/CTLA4), AK-104 (CTLA4/PD-1), MEDI-5752 (CTLA4/PD-1), atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR). In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, atezolizumab, avelumab, envafolimab, durvalumab, cosibelimab, lodapolimab, garivulimab, envafolimab, opucolimab, and manelimab. In some embodiments, the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, and vopratelimab. In some embodiments, the chemotherapy is selected from the group consisting of 5-FU, leucovorin, oxaliplatin, irinotecan, gemcitabine, nab-paclitaxel (Abraxane®), FOLFIRINOX, and combinations thereof. In some embodiments, therapeutic agents used to treat pancreatic cancer comprise a combination of (a) 5-FU+leucovorin+oxaliplatin+irinotecan, (b) 5-FU+nanoliposomal irinotecan, (c) leucovorin+nanoliposomal irinotecan, or (d) gemcitabine+nab-paclitaxel. In some embodiments, the method further comprises co-administering an additional therapeutic agent or therapeutic modality.

### EXAMPLES

The following examples are offered to illustrate, but not to limit the claimed invention.

### Example 1

### Immunoprecipitation Demonstrates the Presence of Integrin αV and β1 Subunits in Primary Human CAFs

Immunoprecipitation of αV and β1 subunits was conducted on primary human cancer associated fibroblasts (CAFs) from one breast cancer (BRAC), one lung adenocarcinoma (LUAD) and three pancreatic ductal adenocarcinoma (PDAC) donors (BRAC Catalog Number: CAF06; LUAD Catalog Number: CAF07-AD; PDAC Catalog Number: CAF08 all acquired from Vitro Biopharma).

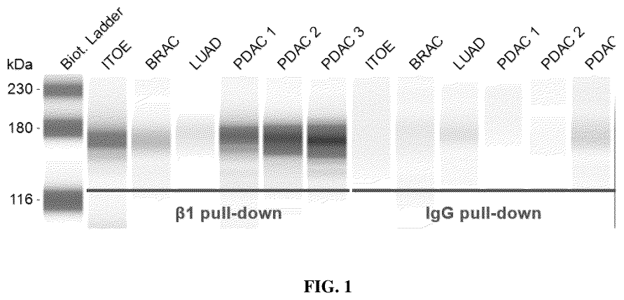

Human primary CAF lysates were generated by lysing the cells in RIPA lysis buffer (Sigma) with Halt™ Protease Inhibitor Cocktail (Thermo Scientific) according to the manufacturer's protocol. Protein concentration was measured by the DC Protein Assay with BSA standard (Bio-Rad). Cell lysates were kept on ice or at 4° C. during the entire procedure. Immunoprecipitation was performed using the Co-IP Kit (Pierce) according to the manufacturer's protocol. In short, 75 g of cell lysates were incubated with anti-Integrin β1 (clone D2E5) antibody or rabbit IgG (control) for 120 minutes and immunoprecipitated following the manufacturer's protocol with the AminoLink Plus Coupling Resin. Proteins immunoprecipitated with Integrin β1 subunit were then immunoblotted with anti-Integrin αV (clone D2N5H) by Wes Separation Module (Protein Simple). Cell line ITOE (CHO cell with integrin β1 subunit overexpressed and α5 subunit knockdown) was used as positive control for Integrin αVβ1 overexpression. Various human primary CAFs were used including: BRAC: Breast Cancer, LUAD: Lung Adenocarcinoma, PDAC: Pancreatic Ductal Adenocarcinoma. FIG. 1 demonstrates the co-expression of integrin av and 11 subunits in breast cancer CAF and pancreatic cancer CAFs.

### Example 2

Combination with Integrin Activity Modulators Enhances Anticancer Activity of Anti-PD-1 Antibody

This example illustrates that co-administration of an integrin activity modulator (e.g., integrin αvβ1/6 inhibitor) can enhance the anticancer activity of an anti-PD-1 antibody.

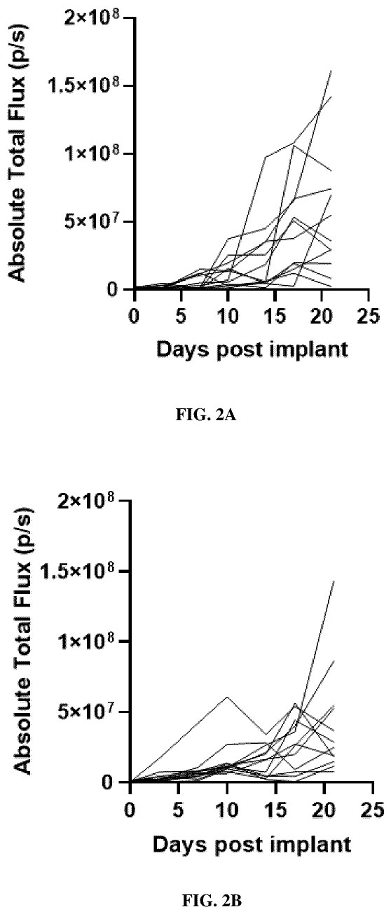

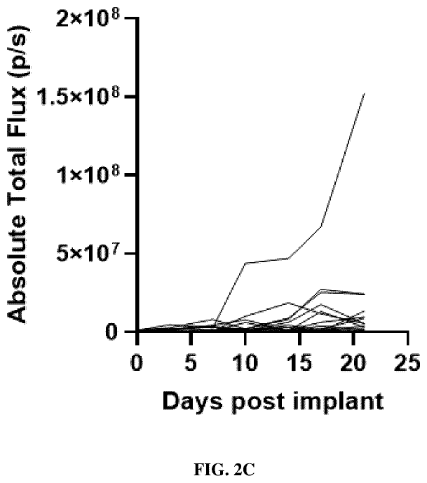

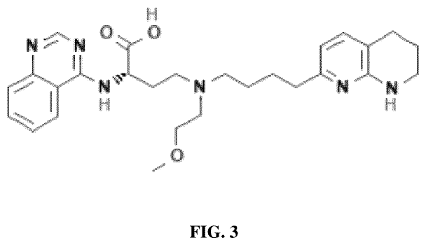

In brief, luciferase labeled tumor chunks derived from LSL-Kras^{G12D/+} p53^{R172H/+} PDX-1Cre mice (model for locally invasive and metastatic pancreatic cancer) were surgically implanted orthotopically into pancreas of C57BL/6 female mice (Crown Biosciences, San Diego). Mice were allowed to recover and 3 days post-implantation, tumors were measured using IVIS imaging, and randomized into various groups. Day 4 post implantation, mice were treated with RatIgG2aK isotype control BIW and a vehicle control BID (Group 1, FIG. 2A), 10 mg/kg of an anti-PD1 antibody (InvivoMab anti-mouse PD-1 (CD279) antibody; J43) and a vehicle control BID (Group 2, FIG. 2B), or 500 mg/kg of integrin activity modulator (bexotegrast, i.e., PLN-74809) and 10 mg/kg of an anti-PD1 antibody (InvivoMab anti-mouse PD-1 (CD279) antibody; J43) (Group 3, FIG. 2C) for 3 weeks. Tumors were measured bi-weekly using IVIS imaging.

Tumor growth curves in FIG. 2A (Group 1), FIG. 2B (Group 2), and FIG. 2C (Group 3) illustrate the results of the above experiment. As shown in FIG. 2B, the anti-PD-1 antibody as a single agent was found to have no to low anticancer activity in the chosen PDAC tumor model. As shown in FIG. 2C, combination of this same anti-PD-1 antibody with an integrin activity modulator was found to result in substantially enhanced antitumor activity.

In sum, this example demonstrates that an integrin activity modulator/anti-PD-1 antibody combination treatment results in much enhanced antitumor activity, relative to a single-agent treatment with the anti-PD-1 antibody.

### Example 3

Evaluation of avb1-Selective Inhibitors

In this example, the efficacy of an ITGαvβ1-selective inhibitor alone or in combination with an ITGαvβ6 inhibitor, an ITGαvβ8 inhibitor, or an anti-PD1 antibody to inhibit tumor growth is evaluated. In addition, the anti-fibrotic potential of the αvβ1-selective inhibitor is evaluated.

In this study, animals from EMT6 and KPC-derived models (EMT6 from ATCC, KPC-derived models from Karafast, U. Pennsylvania Catalog #s EUP013-FP, EUP009-FP, EUP006-FP) are administered the following therapies as shown in Table 1 below.

TABLE 1

| Treatment Group | ITGαvβ1-selective inhibitor¹ | ITGαvβ1/6 inhibitor² | anti-PD1 antibody³ |
|---|---|---|---|
| A | + | | |
| B | | + | |
| C | | | + |
| D | + | | + |
| E | | + | + |

¹ITGαvβ1-selective inhibitor = PLN-1474, disclosed in International Publication No. WO2021127466 as compound 1, having the chemical name (S)-2-(4-methyltetrahydro-2H-pyran-4-carboxamido)-9-(5,6,7,8-tetrahydro-1,8-naphthyridin-2-yl)nonanoic acid, PO administration
²ITGαvβ1/6 inhibitor = PLN-74809, disclosed in International Publication No. WO2019173653 as compound 5, having the chemical name (S)-4-((2-methoxyethyl) (4-(5,6,7,8-tetrahydro-1,8-naphthyridin-2-yl) butyl)amino)-2-(quinazolin-4-ylamino) butanoic acid, PO administration
³anti-PD1 antibody = RMP1-14, BioXCell, Cat #BP0146, IP administration

The antifibrotic impact of integrin inhibition is measured by reduction in Col1a1 transcript by qPCR or nuclear pSMAD by JHC as well as by picrosirius red (PSR) staining and quantification of PSR-positive area.

To evaluate the efficacy of ITGαvβ1-selective versus ITGαvβ1/8 dual inhibitors on tumor growth, animals from EMT6 and KPC-derived models are administered the following therapies as shown in Table 2 below:

TABLE 2

| Treatment | | ITGαvβ1-selective | | |
|---|---|---|---|---|
| Group | anti-ITGαvβ8 antibody¹ | inhibitor² | anti-PD-1 antibody³ | TGFβR1 inhibitor⁴ |
| A | + | | | |
| B | | + | | |
| C | + | + | | |
| D | + | | + | |
| E | | + | + | |
| F | + | + | + | |
| G | + | | + | + |
| H | | + | + | + |
| I | + | + | + | + |
| J | + | | | + |
| K | | + | | + |
| L | | | + | + |
| M | | | + | |
| N | | | | + |

¹anti-ITGαvβ8 antibody = ADWA-11, disclosed in International Publication No. WO2020051333, IP administration
²ITGαvβ1-selective inhibitor = PLN-1474, disclosed in International Publication No. WO2021127466 as compound 1, having the chemical name (S)-2-(4-methyltetrahydro-2H-pyran-4-carboxamido)-9-(5,6,7,8-tetrahydro-1,8-naphthyridin-2-yl)nonanoic acid, PO administration
³anti-PD-1 antibody = RMP1-14, BioXCell, Cat #BP0146, IP administration
⁴TGFβR1 inhibitor = SB 525334, Tocris Cat# 3211, PO administration

Tumor growth is measured by caliper measurements. The antifibrotic impact of integrin and TGFβ3β1 inhibition is measured by reduction in Col1a1 transcript by qPCR or nuclear pSMAD by JHC as well as by picrosirius red (PSR) staining and quantification of PSR-positive area.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of treating, mitigating, reducing, preventing or delaying the recurrence or metastasis of cancer in a subject comprising co-administering to the subject an effective amount of:
a. an integrin activity modulator; and
b. a PD-1 inhibitor or PD-L1 inhibitor.

2. A method for eliciting an immune response to cancer in a subject comprising co-administering to the subject an effective amount of:
a. an integrin activity modulator; and
b. a PD-1 inhibitor or PD-L1 inhibitor.

3. A method of treating, mitigating, reducing, preventing or delaying the recurrence or metastasis of cancer in a subject comprising co-administering to the subject an effective amount of:
a. an integrin activity modulator; and
b. a PD-1 inhibitor or PD-L1 inhibitor,
wherein:
(i) the integrin activity modulator is selected from bexotegrast (i.e., PLN-74809), PLN-1474, and PLN-1561; or
(ii) the PD-1 inhibitor or PD-L1 inhibitor is selected from atezolizumab, durvalumab, MK-7684A, nivolumab, pembrolizumab, and zimberelimab.

4. A method for eliciting an immune response to cancer in a subject comprising co-administering to the subject an effective amount of:
a. an integrin activity modulator; and
b. a PD-1 inhibitor or PD-L1 inhibitor,
wherein:
(i) the integrin activity modulator is selected from bexotegrast (i.e., PLN-74809), PLN-1474, and PLN-1561; or
(ii) the PD-1 inhibitor or PD-L1 inhibitor is selected from atezolizumab, durvalumab, MK-7684A, nivolumab, pembrolizumab, and zimberelimab.

5. A kit for use as a medicament, wherein the kit comprises
a. an integrin activity modulator; and
b. a PD-1 inhibitor or PD-L1 inhibitor.

6. A kit for use in the treatment, mitigation, reduction, prevention, or delay of the recurrence or metastasis of cancer, wherein the kit comprises
a. an integrin activity modulator; and
b. a PD-1 inhibitor or PD-L1 inhibitor.

7. A kit for use as a medicament, wherein the kit comprises
a. an integrin activity modulator; and
b. a PD-1 inhibitor or PD-L1 inhibitor,
wherein:
(i) the integrin activity modulator is selected from bexotegrast (i.e., PLN-74809), PLN-1474, and PLN-1561; or
(ii) the PD-1 inhibitor or PD-L1 inhibitor is selected from atezolizumab, durvalumab, MK-7684A, nivolumab, pembrolizumab, and zimberelimab.

8. A kit for use in the treatment, mitigation, reduction, prevention, or delay of the recurrence or metastasis of cancer, wherein the kit comprises
a. an integrin activity modulator; and
b. a PD-1 inhibitor or PD-L1 inhibitor,
wherein:
(i) the integrin activity modulator is selected from bexotegrast (i.e., PLN-74809), PLN-1474, and PLN-1561; or
(ii) the PD-1 inhibitor or PD-L1 inhibitor is selected from atezolizumab, durvalumab, MK-7684A, nivolumab, pembrolizumab, and zimberelimab.

9. The method of any one of claims 1 to 4 or the kit of any one of claims 5 to 8, wherein the integrin activity modulator is selected from an integrin-activating agent, an integrin-inhibiting agent, or an agent that is capable of interacting with one or more integrins.

10. The method or kit of any one of claims 1 to 9, wherein the integrin activity modulator is a small molecule or a large molecule.

11. The method or kit of claim 10, wherein the large molecule is an antibody or antibody fragment.

12. The method or kit of claim 10, wherein the large molecule is an inhibitory peptide or peptide antagonist.

13. The method or kit of any one of claims 1 to 12, wherein the integrin activity modulator activates, inhibits, or interacts with an integrin alpha ((a) subunit.

14. The method or kit of claim 13, wherein the integrin a subunit is selected from α5, αV, α8, and αIIb.

15. The method or kit of claim 14, wherein the integrin activity modulator activates, inhibits, or interacts with the αV subunit.

16. The method or kit of claim 15, wherein the integrin activity modulator is an anti-αV antibody.

17. The method or kit of claim 16, wherein the anti-αV antibody is selected from abituzumab and intetumumab.

18. The method or kit of claim 15, wherein the integrin activity modulator is an iRGD peptide.

19. The method or kit of claim 18, wherein the iRGD peptide is CEND-1.

20. The method or kit of any one of claims 1 to 19, wherein the integrin activity modulator activates, inhibits, or interacts with an integrin beta (β) subunit.

21. The method or kit of claim 20, wherein the β subunit is selected from β1, β3, β5, and β8.

22. The method or kit of claim 21, wherein the integrin activity modulator activates, inhibits, or interacts with β1.

23. The method or kit of claim 22, wherein the integrin activity modulator is an anti-β1 antibody.

24. The method or kit of claim 23, wherein the anti-β1 antibody is OS2966.

25. The method or kit of claim 21, wherein the integrin activity modulator activates, inhibits, or interacts with β5.

26. The method or kit of claim 25, wherein the integrin activity modulator is an anti-β5 antibody.

27. The method or kit of claim 26, wherein the anti-β5 antibody is ALULA.

28. The method or kit of any one of claims 1 to 27, wherein the integrin activity modulator activates, inhibits, or interacts with an RGD-binding integrin.

29. The method or kit of claim 28, wherein the RGD-binding integrin is selected from α5β1, αVβ1, αVβ3, αVβ5, αVβ6, αVβ8, α8β1, and αIIbβ3.

30. The method or kit of any one of claims 1 to 28, wherein the integrin activity modulator activates, inhibits, or interacts with α5β1.

31. The method or kit of claim 30, wherein the integrin activity modulator is selected from AG-73305, AP25, AMEP, ATN-161, F-200 (Eos), JSM-6427, liposomal contortrostatin, PF-04605412, bexotegrast (i.e., PLN-74809), and SJ-749.

32. The method or kit of any one of claims 1 to 28, wherein the integrin activity modulator activates, inhibits, or interacts with αVβ1.

33. The method or kit of claim 32, wherein the integrin activity modulator is selected from bexotegrast (i.e., PLN-74809), IDL-2965, PLN-1474, PLN-1561, and volociximab.

34. The method or kit of claim 33, wherein the integrin activity modulator is selected from bexotegrast (i.e., PLN-74809), PLN-1474, and PLN-1561.

35. The method or kit of claim 34, wherein the integrin activity modulator is bexotegrast.

36. The method or kit of claim 34, wherein the integrin activity modulator is PLN-1474.

37. The method or kit of claim 34, wherein the integrin activity modulator is PLN-1561.

38. The method or kit of any one of claims 1 to 28, wherein the integrin activity modulator activates, inhibits, or interacts with αVβ3.

39. The method or kit of claim 38, wherein the integrin activity modulator is selected from AP25, B-1451, BS-1417, cilengitide, CT-6725, efatucizumab, etaracizumab, FF-10158, HM-3, IPS-02001, IPS-02101, IPS-05002, JNJ-26076713, MK-0429, NTA-1, NVX-188, Ocurin, ProAgio, PS-388023, RP-697, RP-748, SB-273005, Sch-221153, SC-69000, SD-983, SF-0166, SM-256, SOM-0777, SQ-885, TSRI-265, VIP-236, vitaxin, VPI-2690B, and XT-199.

40. The method or kit of any one of claims 1 to 28, wherein the integrin activity modulator activates, inhibits, or interacts with αVβ5.

41. The method or kit of claim 40, wherein the integrin activity modulator is selected from ALULA, SNAKA-51, and STX-200.

42. The method or kit of any one of claims 1 to 28, wherein the integrin activity modulator activates, inhibits, or interacts with αVβ6.

43. The method or kit of claim 42, wherein the integrin activity modulator is selected from 264-RAD, A20FMDV2, abituzumab, ADVCA0848, bexotegrast (i.e., PLN-74809), CRB-602, GSK-3008348, IDL-2965, intetumumab, MORF-627, MORF-720, PLN-1177, PLN-1561, PLN-1705, SGN-B6A, and STX-100.

44. The method or kit of any one of claims 1 to 28, wherein the integrin activity modulator activates, inhibits, or interacts with αVβ8.

45. The method or kit of claim 44, wherein the integrin activity modulator is selected from CRB-601 and PF-06940434.

46. The method or kit of any one of claims 1 to 28, wherein the integrin activity modulator activates, inhibits, or interacts with α8β1.

47. The method or kit of claim 46, wherein the integrin activity modulator is an anti-α8β1 antibody.

48. The method or kit of any one of claims 1 to 28, wherein the integrin activity modulator activates, inhibits, or interacts with αIIbβ3.

49. The method or kit of claim 48, wherein the integrin activity modulator is selected from eptifibatide, RUC-1, Tc-99m-P280, tirofiban, zalunfiban, and an antibody that interacts with αIIbβ3.

50. The method or kit of any one of claims 1 to 49, wherein the dose of the integrin activity modulator is at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg.

51. The method or kit of any one of claims 1 to 50, wherein the dose of the integrin activity modulator is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg.

52. The method or kit of any one of claims 1 to 51, wherein the dose of the integrin activity modulator is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg.

53. The method or kit of any one of claims 1 to 52, wherein the integrin activity modulator is administered daily.

54. The method or kit of any one of claims 1 to 52, wherein two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 days apart.

55. The method or kit of any one of claims 1 to 52, wherein two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks apart.

56. The method or kit of any one of claims 1 to 52, wherein two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 months apart.

57. The method or kit of any one of claims 1 to 56, wherein the integrin activity modulator is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions.

58. The method or kit of claim 35, wherein the dose of bexotegrast is at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg.

59. The method or kit of claim 35, wherein the dose of bexotegrast is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg.

60. The method or kit of claim 35, wherein the dose of bexotegrast is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg.

61. The method or kit of any one of claims 35 and 58 to 60, wherein bexotegrast is administered daily.

62. The method or kit of any one of claims 35 and 58 to 60, wherein bexotegrast is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions.

63. The method or kit of any one of claims 35 and 58 to 62, wherein bexotegrast is administered orally.

64. The method or kit of any one of claims 1 to 63, wherein the PD-1 inhibitor or PD-L1 inhibitor is an anti-PD-1 antibody.

65. The method or kit of claim 64, wherein the anti-PD-1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), PF-06801591, MGD-019 (PD-1/CTLA4), KN-046 (PD-1/CTLA4), XmAb-20717 (PD-1/CTLA4), AK-104 (CTLA4/PD-1), and MEDI-5752 (CTLA4/PD-1).

66. The method or kit of claim 65, wherein the anti-PD-1 antibody is zimberelimab.

67. The method or kit of any one of claims 1 to 49, wherein the PD-1 inhibitor or PD-L1 inhibitor is an anti-PD-L1 antibody.

68. The method or kit of claim 67, wherein the PD-L1 inhibitor is selected from the group consisting of atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR).

69. The method or kit of any one of claims 1 to 49, wherein the PD-L1 inhibitor is a small molecule inhibitor.

70. The method or kit of claim 69, wherein the small molecule PD-L1 inhibitor is selected from the group consisting of CA-170, GS-4224, GS-4416, INCB99280, INCB99318, and lazertinib.

71. The method or kit of any one of claims 1 to 70, wherein the dose of the PD-1 inhibitor or PD-L1 inhibitor is at least about 30 mg, 60 mg, 120 mg, 240 mg, 360 mg, 480 mg, 600 mg, 720 mg, 840 mg, 960 mg, 1080 mg, or 1200 mg.

72. The method or kit of any one of claims 1 to 70, wherein the dose of the PD-1 inhibitor or PD-L1 inhibitor is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg.

73. The method or kit of any one of claims 1 to 72, wherein the dose of the PD-1 inhibitor or PD-L1 inhibitor is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg.

74. The method or kit of any one of claims 1 to 73, wherein the PD-1 inhibitor or PD-L1 inhibitor is administered daily.

75. The method or kit of any one of claims 1 to 73, wherein two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 days apart.

76. The method or kit of any one of claims 1 to 73, wherein two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks apart.

77. The method or kit of any one of claims 1 to 73, wherein two or more doses of the PD-1 inhibitor or PD-L1 inhibitor are administered at least 3 weeks about.

78. The method or kit of any one of claims 1 to 73, wherein two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 months apart.

79. The method or kit of any one of claims 1 to 78, wherein the PD-1 inhibitor or PD-L1 inhibitor is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularily, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions.

80. The method or kit of claim 66, wherein the dose of zimberelimab is at least about 30 mg, 60 mg, 120 mg, 240 mg, 360 mg, 480 mg, 600 mg, 720 mg, 840 mg, 960 mg, 1080 mg, or 1200 mg.

81. The method or kit of claim 66, wherein the dose of zimberelimab is at least about 360 mg.

82. The method or kit of claim 66 or 80, wherein the dose of zimberelimab is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg.

83. The method or kit of any one of claims 66, 80, or 82, wherein the dose of zimberelimab is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg.

84. The method or kit of any one of claim 66 or 80 to 84, wherein two or more doses of zimberelimab are administered at least about 1, 2, 3, 4, 5, 6, 7, 8, 9 10, 11, or 12 weeks apart.

85. The method or kit of any one of claim 66 or 80 to 84, wherein two or more doses of zimberelimab are administered at least about every 3 weeks.

86. The method or kit of any one of claim 66 or 80 to 85, wherein zimberelimab is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions.

87. The method or kit of any one of claim 66 or 80 to 85, wherein zimberelimab is administered intravenously.

88. The method or kit of any one of claim 66 or 80 to 85, wherein zimberelimab is administered subcutaneously.

89. The method or kit of any one of claims 1 to 88, wherein the cancer comprises a solid tumor.

90. The method or kit of claim 88, wherein the solid tumor is located in or arising from a tissue or organ selected from the group consisting of:
bone (e.g., adamantinoma, aneurysmal bone cysts, angiosarcoma, chondroblastoma, chondroma, chondromyxoid fibroma, chondrosarcoma, chordoma, dedifferentiated chondrosarcoma, enchondroma, epithelioid hemangioendothelioma, fibrous dysplasia of the bone, giant cell tumour of bone, haemangiomas and related lesions, osteoblastoma, osteochondroma, osteosarcoma, osteoid osteoma, osteoma, periosteal chondroma, Desmoid tumor, Ewing sarcoma);
lips and oral cavity (e.g., odontogenic ameloblastoma, oral leukoplakia, oral squamous cell carcinoma, primary oral mucosal melanoma); salivary glands (e.g., pleomorphic salivary gland adenoma, salivary gland adenoid cystic carcinoma, salivary gland mucoepidermoid carcinoma, salivary gland Warthin's tumors);
esophagus (e.g., Barrett's esophagus, dysplasia and adenocarcinoma);
gastrointestinal tract, including stomach (e.g., gastric adenocarcinoma, primary gastric lymphoma, gastrointestinal stromal tumors (GISTs), metastatic deposits, gastric carcinoids, gastric sarcomas, neuroendocrine carcinoma, gastric primary squamous cell carcinoma, gastric adenoacanthomas), intestines and smooth muscle (e.g., intravenous leiomyomatosis), colon (e.g., colorectal adenocarcinoma), rectum, anus;
pancreas (e.g., serous neoplasms, including microcystic or macrocystic serous cystadenoma, solid serous cystadenoma, Von Hippel-Landau (VHL)-associated serous cystic neoplasm, serous cystadenocarcinoma; mucinous cystic neoplasms (MCN), intraductal papillary mucinous neoplasms (IPMN), intraductal oncocytic papillary neoplasms (IOPN), intraductal tubular neoplasms, cystic acinar neoplasms, including acinar cell cystadenoma, acinar cell cystadenocarcinoma, pancreatic adenocarcinoma, invasive pancreatic ductal adenocarcinomas, including tubular adenocarcinoma, adenosquamous carcinoma, colloid carcinoma, medullary carcinoma, hepatoid carcinoma, signet ring cell carcinoma, undifferentiated carcinoma, undifferentiated carcinoma with osteoclast-like giant cells, acinar cell carcinoma, neuroendocrine neoplasms, neuroendocrine microadenoma, neuroendocrine tumors (NET), neuroendocrine carcinoma (NEC), including small cell or large cell NEC, insulinoma, gastrinoma, glucagonoma, serotonin-producing NET, somatostatinoma, VIPoma, solid-pseudopapillary neoplasms (SPN), pancreatoblastoma);
gall bladder (e.g., carcinoma of the gallbladder and extrahepatic bile ducts, intrahepatic cholangiocarcinoma);
neuro-endocrine (e.g., adrenal cortical carcinoma, carcinoid tumors, phaeochromocytoma, pituitary adenomas);
thyroid (e.g., anaplastic (undifferentiated) carcinoma, medullary carcinoma, oncocytic tumors, papillary carcinoma, adenocarcinoma);
liver (e.g., adenoma, combined hepatocellular and cholangiocarcinoma, fibrolamellar carcinoma, hepatoblastoma, hepatocellular carcinoma, mesenchymal, nested stromal epithelial tumor, undifferentiated carcinoma; hepatocellular carcinoma, intrahepatic cholangiocarcinoma, bile duct cystadenocarcinoma, epithelioid hemangioendothelioma, angiosarcoma, embryonal sarcoma, rhabdomyosarcoma, solitary fibrous tumor, teratoma, York sac tumor, carcinosarcoma, rhabdoid tumor);
kidney (e.g., ALK-rearranged renal cell carcinoma, chromophobe renal cell carcinoma, clear cell renal cell carcinoma, clear cell sarcoma, metanephric adenoma, metanephric adenofibroma, mucinous tubular and spindle cell carcinoma, nephroma, nephroblastoma (Wilms tumor), papillary adenoma, papillary renal cell carcinoma, renal oncocytoma, renal cell carcinoma, succinate dehydrogenase-deficient renal cell carcinoma, collecting duct carcinoma);
breast (e.g., invasive ductal carcinoma, including without limitation, acinic cell carcinoma, adenoid cystic carcinoma, apocrine carcinoma, cribriform carcinoma, glycogen-rich/clear cell, inflammatory carcinoma, lipid-rich carcinoma, medullary carcinoma, metaplastic carcinoma, micropapillary carcinoma, mucinous carcinoma, neuroendocrine carcinoma, oncocytic carcinoma, papillary carcinoma, sebaceous carcinoma, secretory breast carcinoma, tubular carcinoma; lobular carcinoma, including without limitation, pleomorphic carcinoma, signet ring cell carcinoma);
peritoneum (e.g., mesothelioma; primary peritoneal cancer);
female sex organ tissues, including ovary (e.g., choriocarcinoma, epithelial tumors, germ cell tumors, sex cord-stromal tumors), Fallopian tubes (e.g., serous adenocarcinoma, mucinous adenocarcinoma, endometrioid adenocarcinoma, clear cell adenocarcinoma, transitional cell carcinoma, squamous cell carcinoma, undifferentiated carcinoma, Müllerian tumors, adenosarcoma, leiomyosarcoma, teratoma, germ cell tumors, choriocarcinoma, trophoblastic tumors), uterus (e.g., carcinoma of the cervix, endometrial polyps, endometrial hyperplasia, intraepithelial carcinoma (EIC), endometrial carcinoma (e.g., endometrioid carcinoma, serous carcinoma, clear cell carcinoma, mucinous carcinoma, squamous cell carcinoma, transitional carcinoma, small cell carcinoma, undifferentiated carcinoma, mesenchymal neoplasia), leiomyoma (e.g., endometrial stromal nodule, leiomyosarcoma, endometrial stromal sarcoma (ESS), mesenchymal tumors), mixed epithelial and mesenchymal tumors (e.g., adenofibroma, carcinofibroma, adenosarcoma, carcinosarcoma (malignant mixed mesodermal sarcoma—MMMT)), endometrial stromal tumors, endometrial malignant mullerian mixed tumours, gestational trophoblastic tumors (partial hydatiform mole, complete hydatiform mole, invasive hydatiform mole, placental site tumour)), vulva, vagina;
male sex organ tissues, including prostate, testis (e.g., germ cell tumors, spermatocytic seminoma), penis;
bladder (e.g., squamous cell carcinoma, urothelial carcinoma, bladder urothelial carcinoma);
brain, (e.g., gliomas (e.g., astrocytomas, including non-infiltrating, low-grade, anaplastic, glioblastomas; oligodendrogliomas, ependymomas), meningiomas, gangliogliomas, schwannomas (neurilemmomas), craniopharyngiomas, chordomas, Non-Hodgkin lymphomas (NHLs), indolent non-Hodgkin's lymphoma (iNHL), refractory iNHL, pituitary tumors;
eye (e.g., retinoma, retinoblastoma, ocular melanoma, posterior uveal melanoma, iris hamartoma);
head and neck (e.g., nasopharyngeal carcinoma, Endolymphatic Sac Tumor (ELST), epidermoid carcinoma, laryngeal cancers including squamous cell carcinoma (SCC) (e.g., glottic carcinoma, supraglottic carcinoma, subglottic carcinoma, transglottic carcinoma), carcinoma in situ, verrucous, spindle cell and basaloid SCC, undifferentiated carcinoma, laryngeal adenocarcinoma, adenoid cystic carcinoma, neuroendocrine carcinomas, laryngeal sarcoma), head and neck paragangliomas (e.g., carotid body, jugulotympanic, vagal);
thymus (e.g., thymoma);
heart (e.g., cardiac myxoma);
lung (e.g., small cell carcinoma (SCLC), non-small cell lung carcinoma (NSCLC), including squamous cell carcinoma (SCC), adenocarcinoma and large cell carcinoma, carcinoids (typical or atypical), carcinosarcomas, pulmonary blastomas, giant cell carcinomas, spindle cell carcinomas, pleuropulmonary blastoma);
lymph (e.g., lymphomas, including Hodgkin's lymphoma, non-Hodgkin's lymphoma (NHL), indolent non-Hodgkin's lymphoma (iNHL), refractory iNHL, Epstein-Barr virus (EBV)-associated lymphoproliferative diseases, including B cell lymphomas and T cell lymphomas (e.g., Burkitt lymphoma; large B cell lymphoma, diffuse large B-cell lymphoma (DLBCL), mantle cell lymphoma, indolent B-cell lymphoma, low grade B cell lymphoma, fibrin-associated diffuse large cell lymphoma; primary effusion lymphoma; plasmablastic lymphoma; extranodal NK/T cell lymphoma, nasal type; peripheral T cell lymphoma, cutaneous T cell lymphoma, angioimmunoblastic T cell lymphoma; follicular T cell lymphoma; systemic T cell lymphoma), lymphangioleiomyomatosis);
central nervous system (CNS) (e.g., gliomas including astrocytic tumors (e.g., pilocytic astrocytoma, pilomyxoid astrocytoma, subependymal giant cell astrocytoma, pleomorphic xanthoastrocytoma, diffuse astrocytoma, fibrillary astrocytoma, gemistocytic astrocytoma, protoplasmic astrocytoma, anaplastic astrocytoma, glioblastoma (e.g., giant cell glioblastoma, gliosarcoma, glioblastoma multiforme) and gliomatosis cerebri), oligodendroglial tumors (e.g., oligodendroglioma, anaplastic oligodendroglioma), oligoastrocytic tumors (e.g., oligoastrocytoma, anaplastic oligoastrocytoma), ependymal tumors (e.g., subependymom, myxopapillary ependymoma, ependymomas (e.g., cellular, papillary, clear cell, tanycytic), anaplastic ependymoma), optic nerve glioma, and non-gliomas (e.g., choroid plexus tumors, neuronal and mixed neuronal-glial tumors, pineal region tumors, embryonal tumors, medulloblastoma, meningeal tumors, primary CNS lymphomas, germ cell tumors, Pituitary adenomas, cranial and paraspinal nerve tumors, stellar region tumors); neurofibroma, meningioma, peripheral nerve sheath tumors, peripheral neuroblastic tumours (including without limitation neuroblastoma, ganglioneuroblastoma, ganglioneuroma), trisomy 19 ependymoma);
neuroendocrine tissues (e.g., paraganglionic system including adrenal medulla (pheochromocytomas) and extra-adrenal paraganglia ((extra-adrenal) paragangliomas);
skin (e.g., clear cell hidradenoma, cutaneous benign fibrous histiocytomas, cylindroma, hidradenoma, melanoma (including cutaneous melanoma, mucosal melanoma), pilomatricoma, Spitz tumors); and
soft tissues (e.g., aggressive angiomyxoma, alveolar rhabdomyosarcoma, alveolar soft part sarcoma, angiofibroma, angiomatoid fibrous histiocytoma, synovial sarcoma, biphasic synovial sarcoma, clear cell sarcoma, dermatofibrosarcoma protuberans, desmoid-type fibromatosis, small round cell tumor, desmoplastic small round cell tumor, elastofibroma, embryonal rhabdomyosarcoma, Ewing's tumors/primitive neurectodermal tumors (PNET), extraskeletal myxoid chondrosarcoma, extraskeletal osteosarcoma, paraspinal sarcoma, inflammatory myofibroblastic tumor, lipoblastoma, lipoma, chondroid lipoma, liposarcoma/malignant lipomatous tumors, liposarcoma, myxoid liposarcoma, fibromyxoid sarcoma, lymphangioleiomyoma, malignant myoepithelioma, malignant melanoma of soft parts, myoepithelial carcinoma, myoepithelioma, myxoinflammatory fibroblastic sarcoma, undifferentiated sarcoma, pericytoma, rhabdomyosarcoma, non-rhabdomyosarcoma soft tissue sarcoma (NRSTS), soft tissue leiomyosarcoma, undifferentiated sarcoma, well-differentiated liposarcoma.

91. The method or kit of any one of claims 1 to 90, wherein the cancer is selected from breast cancer or pancreatic cancer.

92. The method or kit of claim 91, wherein the cancer is breast cancer.

93. The method or kit of claim 92, wherein the breast cancer is triple-negative breast cancer (TNBC), HR+/HER2− breast cancer (HR+/HER2− BC), HR+/HER2low breast cancer (HR+/HER2low BC).

94. The method or kit of claim 91, wherein the cancer is pancreatic cancer.

95. The method or kit of claim 94, wherein the pancreatic cancer is pancreatic ductal adenocarcinoma (PDAC).

96. The method or kit of any one of claims 1 to 95, wherein the cancer is metastatic.

97. The method or kit of any one of claims 1 to 96, wherein the subject is human.

98. The method or kit of any one of claims 1 to 97, wherein the subject is treatment naïve.

99. The method or kit of any one of claims 1 to 97, wherein the subject has received at least one previous anticancer treatment selected from the group consisting of surgery, radiation therapy, chemotherapy, or checkpoint inhibitor therapy.

100. The method or kit of any one of claims 1 to 97, wherein the cancer has progressed or recurred following an anticancer treatment selected from the group consisting of surgery, radiation therapy, chemotherapy, or checkpoint inhibitor therapy.

101. The method or kit of any one of claims 1 to 97, wherein the cancer is resistant or refractory to checkpoint inhibitor therapy.

102. The method or kit of any one of claims 99 to 101, wherein the checkpoint inhibitor therapy comprises an anti-PD-1 antibody or anti-PD-L1 antibody.

103. The method or kit of claim 102, wherein the anti-PD-1 antibody or anti-PD-L1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), MGD-019 (PD-1/CTLA4), KN-046 (PD-1/CTLA4), XmAb-20717 (PD-1/CTLA4), AK-104 (CTLA4/PD-1), MEDI-5752 (CTLA4/PD-1), atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR).

104. The method or kit of claim 103, wherein the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, atezolizumab, avelumab, envafolimab, durvalumab, cosibelimab, lodapolimab, garivulimab, envafolimab, opucolimab, and manelimab.

105. The method or kit of claim 104, wherein the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, and vopratelimab.

106. The method or kit of claim 99 or 100, wherein the chemotherapy is selected from the group consisting of 5-FU, leucovorin, oxaliplatin, irinotecan, gemcitabine, nab-paclitaxel (Abraxane®), FOLFIRINOX, and combinations thereof.

107. The method or kit of any one of claims 1 to 106, wherein the method further comprises co-administering an additional therapeutic agent or therapeutic modality or the kit further comprises an additional therapeutic agent or therapeutic modality.

108. A method of treating, mitigating, reducing, preventing or delaying the recurrence or metastasis of pancreatic cancer or breast cancer in a subject comprising co-administering to the subject an effective amount of:
a. an integrin activity modulator selected from bexotegrast (i.e., PLN-74809), PLN-1474, and PLN-1561; and
b. a PD-1 inhibitor or PD-L1 inhibitor selected from atezolizumab, durvalumab, MK-7684A, nivolumab, pembrolizumab, and zimberelimab.

109. A method for eliciting an immune response to pancreatic cancer or breast cancer in a subject comprising co-administering to the subject an effective amount of:
a. an integrin activity modulator selected from bexotegrast (i.e., PLN-74809), PLN-1474, and PLN-1561; and
b. a PD-1 inhibitor or PD-L1 inhibitor selected from atezolizumab, durvalumab, MK-7684A, nivolumab, pembrolizumab, and zimberelimab.

110. A kit for use as a medicament, wherein the kit comprises
a. an integrin activity modulator selected from bexotegrast (i.e., PLN-74809), PLN-1474, and PLN-1561; and
b. a PD-1 inhibitor or PD-L1 inhibitor selected from atezolizumab, durvalumab, MK-7684A, nivolumab, pembrolizumab, and zimberelimab.

111. A kit for use in the treatment, mitigation, reduction, prevention, or delay of the recurrence or metastasis of pancreatic cancer or breast cancer, wherein the kit comprises
a. an integrin activity modulator selected from bexotegrast (i.e., PLN-74809), PLN-1474, and PLN-1561; and
b. a PD-1 inhibitor or PD-L1 inhibitor selected from atezolizumab, durvalumab, MK-7684A, nivolumab, pembrolizumab, and zimberelimab.

112. The method of claim 108 or 109 or the kit of claim 110 or 111, wherein the dose of the integrin activity modulator is at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg.

113. The method of claim 108 or 109 or the kit of claim 110 or 111, wherein the dose of the integrin activity modulator is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg.

114. The method of claim 108 or 109 or the kit of claim 110 or 111, wherein the dose of the integrin activity modulator is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg.

115. The method or kit of any one of claims 108 to 114, wherein the integrin activity modulator is administered daily.

116. The method or kit of any one of claims 108 to 114, wherein two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 days apart.

117. The method or kit of any one of claims 108 to 114, wherein two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks apart.

118. The method or kit of any one of claims 108 to 114, wherein two or more doses of the integrin activity modulator are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 months apart.

119. The method or kit of any one of claims 108 to 118, wherein the integrin activity modulator is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions.

120. The method or kit of any one of claims 108 to 119, wherein the integrin activity modulator is bexotegrast.

121. The method or kit of claim 120, wherein the dose of bexotegrast is at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg.

122. The method or kit of claim 120, wherein the dose of bexotegrast is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg.

123. The method or kit of claim 120, wherein the dose of bexotegrast is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg.

124. The method or kit of any one of claims 120 to 123, wherein bexotegrast is administered daily.

125. The method or kit of any one of claims 120 to 124, wherein bexotegrast is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions.

126. The method or kit of any one of claims 120 to 124, wherein bexotegrast is administered orally.

127. The method or kit of any one of claims 108 to 119, wherein the integrin activity modulator is PLN-1474.

128. The method or kit of any one of claims 108 to 119, wherein the integrin activity modulator is PLN-1561.

129. The method or kit of any one of claims 108 to 128, wherein the dose of the PD-1 inhibitor or PD-L1 inhibitor is at least about 30 mg, 60 mg, 120 mg, 240 mg, 360 mg, 480 mg, 600 mg, 720 mg, 840 mg, 960 mg, 1080 mg, or 1200 mg.

130. The method or kit of any one of claims 108 to 128, wherein the dose of the PD-1 inhibitor or PD-L1 inhibitor is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg.

131. The method or kit of any one of claims 108 to 128, wherein the dose of the PD-1 inhibitor or PD-L1 inhibitor is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg.

132. The method or kit of any one of claims 108 to 131, wherein the PD-1 inhibitor or PD-L1 inhibitor is administered daily.

133. The method or kit of any one of claims 108 to 131, wherein two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 days apart.

134. The method or kit of any one of claims 108 to 131, wherein two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks apart.

135. The method or kit of any one of claims 108 to 131, wherein two or more doses of the PD-1 inhibitor or PD-L1 inhibitor are administered at least 3 weeks about.

136. The method or kit of any one of claims 108 to 131, wherein two or more doses of the PD-1 inhibitor or PD-L1 inhibitor are administered at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 months apart.

137. The method or kit of any one of claims 108 to 136, wherein the PD-1 inhibitor or PD-L1 inhibitor is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularily, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions.

138. The method or kit of any one of claims 108 to 137, wherein the PD-1 inhibitor or PD-L1 inhibitor is atezolizumab.

139. The method or kit of any one of claims 108 to 137, wherein the PD-1 inhibitor or PD-L1 inhibitor is durvalumab.

140. The method or kit of any one of claims 108 to 137, wherein the PD-1 inhibitor or PD-L1 inhibitor is MK-7684A.

141. The method or kit of any one of claims 108 to 137, wherein the PD-1 inhibitor or PD-L1 inhibitor is nivolumab.

142. The method or kit of any one of claims 108 to 137, wherein the PD-1 inhibitor or PD-L1 inhibitor is pembrolizumab.

143. The method or kit of any one of claims 108 to 137, wherein the PD-1 inhibitor or PD-L1 inhibitor is zimberelimab.

144. The method or kit of claim 143, wherein the dose of zimberelimab is at least about 30 mg, 60 mg, 120 mg, 240 mg, 360 mg, 480 mg, 600 mg, 720 mg, 840 mg, 960 mg, 1080 mg, or 1200 mg.

145. The method or kit of claim 143, wherein the dose of zimberelimab is at least about 360 mg.

146. The method or kit of claim 144, wherein the dose of zimberelimab is less than about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3500 mg, 4000, 4500 mg, 5000 mg, 5500 mg, 6000 mg, 6500 mg, 7000 mg, 7500 mg, 8000 mg, 8500 mg, 9000 mg, 9500 mg, or 10000 mg.

147. The method or kit of any one of claims 143 to 146, wherein the dose of zimberelimab is between about 5 mg to about 500 mg, about 10 mg to about 500 mg, about 20 mg to about 500 mg, about 30 mg to about 500 mg, about 40 mg to about 500 mg, 5 mg to about 400 mg, about 10 mg to about 400 mg, about 20 mg to about 400 mg, about 30 mg to about 400 mg, about 40 mg to about 400 mg, 5 mg to about 300 mg, about 10 mg to about 300 mg, about 20 mg to about 300 mg, about 30 mg to about 300 mg, about 40 mg to about 300 mg, 5 mg to about 200 mg, about 10 mg to about 200 mg, about 20 mg to about 200 mg, about 30 mg to about 200 mg, or about 40 mg to about 200 mg.

148. The method or kit of any one of claims 143 to 147, wherein two or more doses of zimberelimab are administered at least about 1, 2, 3, 4, 5, 6, 7, 8, 9 10, 11, or 12 weeks apart.

149. The method or kit of any one of claims 143 to 147, wherein two or more doses of zimberelimab are administered at least about every 3 weeks.

150. The method or kit of any one of claims 143 to 149, wherein zimberelimab is administered subcutaneously, intravenously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions.

151. The method or kit of any one of claims 143 to 149, wherein zimberelimab is administered intravenously.

152. The method or kit of any one of claims 143 to 149, wherein zimberelimab is administered subcutaneously.

153. The method of claim 108 or 109 or the kit of claim 110 or 111, wherein the breast cancer is triple-negative breast cancer (TNBC), HR+/HER2− breast cancer (HR+/HER2− BC), HR+/HER2low breast cancer (HR+/HER2low BC).

154. The method of claim 108 or 109 or the kit of claim 110 or 111, wherein the pancreatic cancer is pancreatic ductal adenocarcinoma (PDAC).

155. The method or kit of any one of claims 108 to 154, wherein the cancer is metastatic.

156. The method or kit of any one of claims 108 to 155, wherein the subject is human.

157. The method or kit of any one of claims 108 to 156, wherein the subject is treatment naïve.

158. The method or kit of any one of claims 108 to 156, wherein the subject has received at least one previous anticancer treatment selected from the group consisting of surgery, radiation therapy, chemotherapy, or checkpoint inhibitor therapy.

159. The method or kit of any one of claims 108 to 156, wherein the cancer has progressed or recurred following an anticancer treatment selected from the group consisting of surgery, radiation therapy, chemotherapy, or checkpoint inhibitor therapy.

160. The method or kit of any one of claims 108 to 156, wherein the cancer is resistant or refractory to checkpoint inhibitor therapy.

161. The method or kit of any one of claims 158 to 160, wherein the checkpoint inhibitor therapy comprises an anti-PD-1 antibody or anti-PD-L1 antibody.

162. The method or kit of claim 161, wherein the anti-PD-1 antibody or anti-PD-L1 antibody is selected from the group consisting of zimberelimab, pembrolizumab, nivolumab, cemiplimab, pidilizumab, MEDI0680, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, AK-105, CS-1003, BI-754091, LZM-009, Sym-021, BAT-1306, PD1-PIK, tebotelimab (PD-1/LAG-3), RG-6139 (PD-1/LAG-3), FS-118 (LAG-3/PD-L1), RO-7121661 (PD-1/TIM-3), RG7769 (PD-1/TIM-3), TAK-252 (PD-1/OX40L), PF-06936308 (PD-1/CTLA4), MGD-019 (PD-1/CTLA4), KN-046 (PD-1/CTLA4), XmAb-20717 (PD-1/CTLA4), AK-104 (CTLA4/PD-1), MEDI-5752 (CTLA4/PD-1), atezolizumab, avelumab, envafolimab, durvalumab, cosibelimab, lodapolimab, garivulimab, envafolimab, opucolimab, manelimab, CX-072, CBT-502, MSB-2311, SHR-1316, sugemalimab, A167, STI-A1015, FAZ-053, BMS-936559, INCB086550, GEN-1046 (PD-L1/4-1BB), FPT-155 (CTLA4/PD-L1/CD28), bintrafusp alpha (M7824; PD-L1/TGFβ-EC domain), CA-170 (PD-L1/VISTA), CDX-527 (CD27/PD-L1), LY-3415244 (TIM-3/PDL1), INBRX-105 (4-1BB/PDL1), MAX10181 and GNS-1480 (PD-L1/EGFR).

163. The method or kit of claim 161, wherein the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, vopratelimab, atezolizumab, avelumab, envafolimab, durvalumab, cosibelimab, lodapolimab, garivulimab, envafolimab, opucolimab, and manelimab.

164. The method or kit of claim 161, wherein the anti-PD-1 antibody or anti-PD-L1 antibody is selected from the group consisting of zimberelimab, pembrolizumab, nivolumab, cemiplimab, pidilizumab, spartalizumab, tislelizumab, toripalimab, genolimzumab, camrelizumab, sintilimab, dostarlimab, sasanlimab, cetrelimab, serplulimab, balstilimab, prolgolimab, budigalimab, and vopratelimab.

165. The method or kit of claim 158 or 159, wherein the chemotherapy is selected from the group consisting of 5-FU, leucovorin, oxaliplatin, irinotecan, gemcitabine, nab-paclitaxel (Abraxane®), FOLFIRINOX, and combinations thereof. In some embodiments, therapeutic agents used to treat pancreatic cancer comprise a combination of (a) 5-FU+leucovorin+oxaliplatin+irinotecan, 5-FU+nanoliposomal irinotecan, leucovorin+nanoliposomal irinotecan, and gemcitabine+nab-paclitaxel.

166. The method or kit of any one of claims 108 to 165, wherein the method further comprises co-administering an additional therapeutic agent or therapeutic modality or the kit further comprises an additional therapeutic agent or therapeutic modality.
